SEQUENCE LISTING (28 sequences)

SEQ ID NO: 1 - DNA, 20 bp, synthetic construct
  source 1..20: mol_type = other DNA; note = B_Frag_upaY_F
cgctcggacaaagaaggacc

SEQ ID NO: 2 - DNA, 21 bp, synthetic construct
  source 1..21: mol_type = other DNA; note = B_Frag_upaY_R
acttctaccctacgacgacga

SEQ ID NO: 3 - DNA, 21 bp, synthetic construct
  source 1..21: mol_type = other DNA; note = B_Frag_PSA_M
ggtgttccaaaagacgaacgt

SEQ ID NO: 4 - DNA, 24 bp, synthetic construct
  source 1..24: mol_type = other DNA; note = B_Frag_PSA_F
tgtgtaaatgataggaggctaggg

SEQ ID NO: 5 - DNA, 20 bp, synthetic construct
  source 1..20: mol_type = other DNA; note = rpsL_F
ccgaactctgcaatgcgtaa

SEQ ID NO: 6 - DNA, 20 bp, synthetic construct
  source 1..20: mol_type = other DNA; note = rpsL_R
cgcgaaccagtacgattgag

SEQ ID NO: 7 - DNA, 22 bp, synthetic construct
  source 1..22: mol_type = other DNA; note = Type1RM_hsdS_F
gacaatcgagatgaagaacaac

SEQ ID NO: 8 - DNA, 21 bp, synthetic construct
  source 1..21: mol_type = other DNA; note = Type1RM_hsdS_R
ccataggcgtatgatttcctg

SEQ ID NO: 9 - DNA, 673 bp, synthetic construct
  source 1..673: mol_type = other DNA; note = Engineered sequence of the bacteriophage including homology arms and 108bp insert recognition site for the 57/59 orientation
ccgcgcctagcacctgccgaggccgccaaggtacccgccataccaccggaacaccttagataaccaagcgtcaccgaacactgacatccgttcaattacacccgtctttcgggcaaagttaacacaaaatgccgctacaaacgaaacaagtagcacgcaagaaagaaaataattataaatctccataattcaaatattagtttgtacaaaagtatgaataaaaagcaattttgcaaagatattgattattaaaacgttagtctcgaactctccgaaggtggataagacatgttgatctttcgtgttacgggatcaatgtcaacacgtcgaccagttttaaaatcgataaagcctttttcaacttgtggcgcttgaaattgaatgatccttctgtcaaacgaaatgtttataaataagaaaaggagtctaaaagactcctaacctacatgctttctgtataaaacgaacgcttccacaatctccggcataactttaagcgatccaacgataagccgccttttaatcatagacgaaataatgataatcttgtcttcttcacaccgaatatatcccatcttatcagcccacgttataaaatcaaacatttcatgttcaaacaacaaagagcctaaaggaaggtatattggatccagcatgcgcaacgcctgcgagc

SEQ ID NO: 10 - DNA, 110 bp, synthetic construct
  source 1..110: mol_type = other DNA; note = Engineered sequence, the 108bp insert with two recognition sites for the 57/59 orientation
gacatgttgatctttcgtgttacgggatcaatgtcaacacgtcgaccagttttaaaatcgataaagcctttttcaacttgtggcgcttgaaattgaatgatccttctgtc

SEQ ID NO: 11 - DNA, 671 bp, synthetic construct
  source 1..671: mol_type = other DNA; note = Engineered sequence of the bacteriophage including homology arms and 108bp insert recognition site for the 57/58 orientation
ccgcgcctagcacctgccgaggccgccaaggtacccgccataccaccggaacaccttagataaccaagcgtcaccgaacactgacatccgttcaattacacccgtctttcgggcaaagttaacacaaaatgccgctacaaacgaaacaagtagcacgcaagaaagaaaataattataaatctccataattcaaatattagtttgtacaaaagtatgaataaaaagcaattttgcaaagatattgattattaaaacgttagtctcgaactctccgaaggtggataagacatgttctgtttcgtgttacgggatcaatgtcaacacgtcgaccagttttaaaatcgataaagcctttttcaacttgtggcgcttgaaattgaatcagcttctgtcaaacgaaatgtttataaataagaaaaggagtctaaaagactcctaacctacatgctttctgtataaaacgaacgcttccacaatctccggcataactttaagcgatccaacgataagccgccttttaatcatagacgaaataatgataatcttgtcttcttcacaccgaatatatcccatcttatcagcccacgttataaaatcaaacatttcatgttcaaacaacaaagagcctaaaggaaggtatattggatccagcatgcgcaacgcctgcgagc

SEQ ID NO: 12 - DNA, 108 bp, synthetic construct
  source 1..108: mol_type = other DNA; note = Engineered sequence, the 108bp insert with two recognition sites for the 57/58 orientation
gacatgttctgtttcgtgttacgggatcaatgtcaacacgtcgaccagttttaaaatcgataaagcctttttcaacttgtggcgcttgaaattgaatcagcttctgtc

SEQ ID NO: 13 - DNA, 1080 bp, synthetic construct
  source 1..1080: mol_type = other DNA; note = Nucleic acid sequence encoding the B. fragilis S-protein orientation 57-57
aatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaagctaatagatgagcgtatcgcaacccaaaacaaaataatagctcatttagaatctctaattaaagggcttacaaaccaacttctgattccaaatagcaattggcaaccaactactatcggacaagtactaacgatcaatccaggtaaggactataagcaccttaaagaaggaaatattccagtttatggtacaggtggctatatgctttctgtaaatgactatttatatgacggtgaaagtgtgtgtattgggcgcaagggtacaattaataagccaatctttttaactggaaaattttggactatagatacgcttttttatacctccaatttcaattcattattaccaagattcggttattatttatttaagaccattgattggctcaagtataatgaagcttcaggtgtcccaagtttatccaaggtatcgatagaaaaaattcatatttcattaccatcattagcaatacaaaacagcatatgccgcttattagattcaatatatgataaattggctttagaggaatctgtattaaataatcaccaaactcaaaaggcgtttatactccaacaaatgtttatatga

SEQ ID NO: 14 - DNA, 492 bp, synthetic construct
  source 1..492: mol_type = other DNA; note = Nucleic acid sequence encoding the B. fragilis S-protein Head 57
aatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaag

SEQ ID NO: 15 - DNA, 550 bp, synthetic construct
  source 1..550: mol_type = other DNA; note = Nucleic acid sequence encoding the B. fragilis S-protein Tail 57
agctcatttagaatctctaattaaagggcttacaaaccaacttctgattccaaatagcaattggcaaccaactactatcggacaagtactaacgatcaatccaggtaaggactataagcaccttaaagaaggaaatattccagtttatggtacaggtggctatatgctttctgtaaatgactatttatatgacggtgaaagtgtgtgtattgggcgcaagggtacaattaataagccaatctttttaactggaaaattttggactatagatacgcttttttatacctccaatttcaattcattattaccaagattcggttattatttatttaagaccattgattggctcaagtataatgaagcttcaggtgtcccaagtttatccaaggtatcgatagaaaaaattcatatttcattaccatcattagcaatacaaaacagcatatgccgcttattagattcaatatatgataaattggctttagaggaatctgtattaaataatcaccaaactcaaaaggcgtttatactccaacaaatgtttatatga

SEQ ID NO: 16 - DNA, 1136 bp, synthetic construct
  source 1..1136: mol_type = other DNA; note = Nucleic acid sequence encoding the B. fragilis S-protein orientation 57-58
aatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaagaatctctaattaaagggctttctgatactcttttagagaatcctctgtggcaaaagacttatctgcgctcatttatgcaattctactcgactaattcattgtcatgggggcagctaagctatgaagaaggtgaaattagaaatctccactatggcttaatacatagtttccaaacaagtgaaattgatactgattctttaccaaggattctgccagccaacgttcccaaacaatacacattatgtcaggctggtgatgtggcttttgctgatgcctcagaagatacggatgagattggtaaagccgtagaatttattagaacacacaaagcaagtgtaatatgtggcttacatactattcatggaagagatataaagtgcaaaacccttcttggtttcaagagggttgcattcaattctcattatttccatgaccaaatcaagcgattggctcaaggaacaaaagtgttctctataacttcaagtaatctgtcatcgtgctatatctatattcctgatatagtcatgcaaaagtccattgtagttttatttgaagcttacgaagaacaacttataacgaacaaaaggttactagaacaatacgaaaaacagaaaaggtatttacttcagcagatgttcatataa

SEQ ID NO: 17 - DNA, 624 bp, synthetic construct
  source 1..624: mol_type = other DNA; note = Nucleic acid sequence encoding the B. fragilis S-protein Tail 58
tctgatactcttttagagaatcctctgtggcaaaagacttatctgcgctcatttatgcaattctactcgactaattcattgtcatgggggcagctaagctatgaagaaggtgaaattagaaatctccactatggcttaatacatagtttccaaacaagtgaaattgatactgattctttaccaaggattctgccagccaacgttcccaaacaatacacattatgtcaggctggtgatgtggcttttgctgatgcctcagaagatacggatgagattggtaaagccgtagaatttattagaacacacaaagcaagtgtaatatgtggcttacatactattcatggaagagatataaagtgcaaaacccttcttggtttcaagagggttgcattcaattctcattatttccatgaccaaatcaagcgattggctcaaggaacaaaagtgttctctataacttcaagtaatctgtcatcgtgctatatctatattcctgatatagtcatgcaaaagtccattgtagttttatttgaagcttacgaagaacaacttataacgaacaaaaggttactagaacaatacgaaaaacagaaaaggtatttacttcagcagatgttcatataa

SEQ ID NO: 18 - DNA, 1132 bp, synthetic construct
  source 1..1132: mol_type = other DNA; note = Nucleic acid sequence encoding the B. fragilis S-protein orientation 57-59
aatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaagctaatagacgagcgtatcgcaacccaaaacaaaataattgaggagtataaaaaattaaagaatgcacttgcggaaagtctgtattatccaaaaaagagtcaagaattgttaaaactcaaggggcttaatagtaaatgggaacaatgctttttgaaagatgtagttgagaacttctgtcgtagaaataaatcgcatatacaataccctatgtattccgtgacaaatgacttgggctttgtgccacagtcggaaaagtttgaagaaagaactatgatgggtgaagatatatcttcttataaagttattaacaagggtgattttgcctataatcccgcaaggataaatgttggttcaatagctaaatatgagggagataatccttgtatgattagttctctatacgtttgctttagacctaaatacaatatatcttccgaatggctccaacacttactcaagtcgcaaaggatgatatacaactataatttatttggtgaaggtggggtaaggatatatctattctttccaaactttggacgaattaagataagtataccacctctcgaagaacaaaaaaaaattgcggcagtaatttcaactatcgaacaaaaaatatcagttgagaactttattcttgata

SEQ ID NO: 19 - DNA, 581 bp, synthetic construct
  source 1..581: mol_type = other DNA; note = Nucleic acid sequence encoding the B. fragilis S-protein Tail 59
gaatgcacttgcggaaagtctgtattatccaaaaaagagtcaagaattgttaaaactcaaggggcttaatagtaaatgggaacaatgctttttgaaagatgtagttgagaacttctgtcgtagaaataaatcgcatatacaataccctatgtattccgtgacaaatgacttgggctttgtgccacagtcggaaaagtttgaagaaagaactatgatgggtgaagatatatcttcttataaagttattaacaagggtgattttgcctataatcccgcaaggataaatgttggttcaatagctaaatatgagggagataatccttgtatgattagttctctatacgtttgctttagacctaaatacaatatatcttccgaatggctccaacacttactcaagtcgcaaaggatgatatacaactataatttatttggtgaaggtggggtaaggatatatctattctttccaaactttggacgaattaagataagtataccacctctcgaagaacaaaaaaaaattgcggcagtaatttcaactatcgaacaaaaaatatcagttgagaactttattcttgata

SEQ ID NO: 20 - DNA, 1119 bp, synthetic construct
  source 1..1119: mol_type = other DNA; note = Nucleic acid sequence encoding the B. fragilis S-protein orientation 57-60
aatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaagctaatagacgagcgtatcgcaacccaaaacaaaataattgaggagtataaaaaattaaagaatgcacttgcggaattattttttgcaaaatctatcgaatatacttcaattggtgaaatgtgtgatgttgttatggggcagtccccatcctcagtggcatataattacacaaagaatgggcttccattgattcaaggaaacttagatattttcgaaggtgttacttctccaagaatgtggacaagtgacataactaagcaatgtgatataggtgatattatacttacagtgcgagctcctgtcggggatgttgccaagtctaatatgattgcctgtgtgggtagaggggtttgtgctattaaagtcaaagaatctggatgtagcgaatatgtatatcaatatttactgtactttaaagctaaatggggctctattgagcaaggaagcacattttcggcaataagtcgtaatgatattctaaatattaacattccggttattacaaaaaggctgattgttgcaagtcatcttttagctttgtttgacagtgaaatcagtatagaagcattaaacttaaacgtatatacaaagcagaaacagtatttgctaaccaagatgtttatataa

SEQ ID NO: 21 - DNA, 589 bp, synthetic construct
  source 1..589: mol_type = other DNA; note = Nucleic acid sequence encoding the B. fragilis S-protein Tail 60
tgaggagtataaaaaattaaagaatgcacttgcggaattattttttgcaaaatctatcgaatatacttcaattggtgaaatgtgtgatgttgttatggggcagtccccatcctcagtggcatataattacacaaagaatgggcttccattgattcaaggaaacttagatattttcgaaggtgttacttctccaagaatgtggacaagtgacataactaagcaatgtgatataggtgatattatacttacagtgcgagctcctgtcggggatgttgccaagtctaatatgattgcctgtgtgggtagaggggtttgtgctattaaagtcaaagaatctggatgtagcgaatatgtatatcaatatttactgtactttaaagctaaatggggctctattgagcaaggaagcacattttcggcaataagtcgtaatgatattctaaatattaacattccggttattacaaaaaggctgattgttgcaagtcatcttttagctttgtttgacagtgaaatcagtatagaagcattaaacttaaacgtatatacaaagcagaaacagtatttgctaaccaagatgtttatataa

SEQ ID NO: 22 - DNA, 1596 bp, synthetic construct
  source 1..1596: mol_type = other DNA; note = Nucleic acid sequence encoding the B. fragilis S-protein orientation 60-57
aatttcccaactttgcgatttccagagttctcgggtgagtggaaaaagtatttcataagagacattgcagaggtcacgaagggggcgggtatatctaaagaacaacgttcgctttttggaactccttgcattttatatggagagttatatacaacctataaatcagaggtaataaatgatgtccaaagcaaaacagatattgacgcgaaaaatttggttagaagcaaagagaatgatgtaataataccatcttcaggtgaaaccgctattgatatctcaacagccagatgtgtaccttatgacgatgttttgttaggcggagacttaaacatcataaggttatatcaaaatgatgggcgttttctgagttatcaattgaatggtgttcgtaaattagatattgcaagggttgcgcaaggaagttcggttattcacctatatggagaaagtataaaatctctatctgtgtctctacctgcactaaaagagcaacaaaagatagtgtcacttctatccaatttcccaactttgcgatttccagagttctcgggtgagtggaaaaaagatattattggtaatgttatatctgttaaatcggagaaatataaccctcattccaataggacagaatttatatgcgtggaattagagagtattagtcaagggacgggcgaattgttggagacctttaactccgcagaacaaaaaagcattaaaaataagttctctccaggaactgtattatttggtaagctaagaccatatcttagaaaattctatttgccttattttgaaggagtttgttcgtctgaaatatgggtaatgagaagcaataaaattgaacctgcctttttgtatagttttattcaaaccccatattttattagtcttgcgaaccaatcaagtggctctaaaatgccaagagccgactgggggttaattgaaacctcaaaaatagcatacccacccaatagtgcagaaagagttaagataggaaagttcttgaagctaatagatgagcgtatcgcaacccaaaacaaaataatagctcatttagaatctctaattaaagggcttacaaaccaacttctgattccaaatagcaattggcaaccaactactatcggacaagtactaacgatcaatccaggtaaggactataagcaccttaaagaaggaaatattccagtttatggtacaggtggctatatgctttctgtaaatgactatttatatgacggtgaaagtgtgtgtattgggcgcaagggtacaattaataagccaatctttttaactggaaaattttggactatagatacgcttttttatacctccaatttcaattcattattaccaagattcggttattatttatttaagaccattgattggctcaagtataatgaagcttcaggtgtcccaagtttatccaaggtatcgatagaaaaaattcatatttcattaccatcattagcaatacaaaacagcatatgccgcttattagattcaatatatgataaattggctttagaggaatctgtattaaataatcaccaaactcaaaaggcgtttatactccaacaaatgtttatatga

SEQ ID NO: 23 - DNA, 516 bp, synthetic construct
  source 1..516: mol_type = other DNA; note = Nucleic acid sequence encoding the B. fragilis S-protein Head 60
aatttcccaactttgcgatttccagagttctcgggtgagtggaaaaagtatttcataagagacattgcagaggtcacgaagggggcgggtatatctaaagaacaacgttcgctttttggaactccttgcattttatatggagagttatatacaacctataaatcagaggtaataaatgatgtccaaagcaaaacagatattgacgcgaaaaatttggttagaagcaaagagaatgatgtaataataccatcttcaggtgaaaccgctattgatatctcaacagccagatgtgtaccttatgacgatgttttgttaggcggagacttaaacatcataaggttatatcaaaatgatgggcgttttctgagttatcaattgaatggtgttcgtaaattagatattgcaagggttgcgcaaggaagttcggttattcacctatatggagaaagtataaaatctctatctgtgtctctacctgcactaaaagagcaacaaaagatagtgtcacttctatcc

SEQ ID NO: 24 - DNA, 1080 bp, synthetic construct; identical to SEQ ID NO: 13
  source 1..1080: mol_type = other DNA; note = Nucleic acid sequence encoding the B. fragilis S-protein Tail 57

SEQ ID NO: 25 - DNA, 1209 bp, synthetic construct
  source 1..1209: mol_type = other DNA; note = Nucleic acid sequence encoding the B. fragilis S-protein orientation 60-58
aatttcccaactttgcgatttccagagttctcgggtgagtggaaaaagtatttcataagagacattgcagaggtcacgaagggggcgggtatatctaaagaacaacgttcgctttttggaactccttgcattttatatggagagttatatacaacctataaatcagaggtaataaatgatgtccaaagcaaaacagatattgacgcgaaaaatttggttagaagcaaagagaatgatgtaataataccatcttcaggtgaaaccgctattgatatctcaacagccagatgtgtaccttatgacgatgttttgttaggcggagacttaaacatcataaggttatatcaaaatgatgggcgttttctgagttatcaattgaatggtgttcgtaaattagatattgcaagggttgcgcaaggaagttcggttattcacctatatggagaaagtataaaatctctatctgtgtctctacctgcactaaaagagcaacaaaagatagtgtcacttctatccctaatagacgagcgtatcgcaacccaaaacaaaataatagctcatttagaatctctaattaaagggctttctgatactcttttagagaatcctctgtggcaaaagacttatctgcgctcatttatgcaattctactcgactaattcattgtcatgggggcagctaagctatgaagaaggtgaaattagaaatctccactatggcttaatacatagtttccaaacaagtgaaattgatactgattctttaccaaggattctgccagccaacgttcccaaacaatacacattatgtcaggctggtgatgtggcttttgctgatgcctcagaagatacggatgagattggtaaagccgtagaatttattagaacacacaaagcaagtgtaatatgtggcttacatactattcatggaagagatataaagtgcaaaacccttcttggtttcaagagggttgcattcaattctcattatttccatgaccaaatcaagcgattggctcaaggaacaaaagtgttctctataacttcaagtaatctgtcatcgtgctatatctatattcctgatatagtcatgcaaaagtccattgtagttttatttgaagcttacgaagaacaacttataacgaacaaaaggttactagaacaatacgaaaaacagaaaaggtatttacttcagcagatgttcatataa

SEQ ID NO: 26 - DNA, 1763 bp, synthetic construct
  source 1..1763: mol_type = other DNA; note = Nucleic acid sequence encoding the B. fragilis S-protein orientation 60-59
aatttcccaactttgcgatttccagagttctcgggtgagtggaaaaagtatttcataagagacattgcagaggtcacgaagggggcgggtatatctaaagaacaacgttcgctttttggaactccttgcattttatatggagagttatatacaacctataaatcagaggtaataaatgatgtccaaagcaaaacagatattgacgcgaaaaatttggttagaagcaaagagaatgatgtaataataccatcttcaggtgaaaccgctattgatatctcaacagccagatgtgtaccttatgacgatgttttgttaggcggagacttaaacatcataaggttatatcaaaatgatgggcgttttctgagttatcaattgaatggtgttcgtaaattagatattgcaagggttgcgcaaggaagttcggttattcacctatatggagaaagtataaaatctctatctgtgtctctacctgcactaaaagagcaacaaaagatagtgtcacttctatccctaatagacgagcgtatcgcaacccaaaacaaaataattgaggagtataaaaaattaaagaatgcacttgcggaaagtctgtattatccaaaaaagagtcaagaattgttaaaactcaaggggcttaatagtaaatgggaacaatgctttttgaaagatgtagttgagaacttctgtcgtagaaataaatcgcatatacaataccctatgtattccgtgacaaatgacttgggctttgtgccacagtcggaaaagtttgaagaaagaactatgatgggtgaagatatatcttcttataaagttattaacaagggtgattttgcctataatcccgcaaggataaatgttggttcaatagctaaatatgagggagataatccttgtatgattagttctctatacgtttgctttagacctaaatacaatatatcttccgaatggctccaacacttactcaagtcgcaaaggatgatatacaactataatttatttggtgaaggtggggtaaggatatatctattctttccaaactttggacgaattaagataagtataccacctctcgaagaacaaaaaaaaattgcggcagtaatttcaactatcgaacaaaaaatatcagttgagaactttattcttgataaattgaatactcaaaaatcattcttactaaccaagatgtttatataaacatcttggttagcaaatactgtttctgctttgtatatacgtttaagtttaatgcttctatactgatttcactgtcaaacaaagctaaaagatgacttgcaacaatcagcctttttgtaataaccggaatgttaatatttagaatatcattacgacttattgccgaaaatgtgcttccttgctcaatagagccccatttagctttaaagtacagtaaatattgatatacatattcgctacatccagattctttgactttaatagcacaaacccctctacccacacaggcaatcatattagacttggcaacatccccgacaggagctcgcactgtaagtataatatcacctatatcacattgcttagttatgtcacttgtccacattcttggagaagtaacaccttcgaaaatatctaagtttccttgaatcaatggaagcccattctttgtgtaattatatgccactgaggatggggactgccccataacaacatcacacatttcaccaattgaagtatattcgatagattttgcaaaaaataattccgcaagtgcattc

SEQ ID NO: 27 - DNA, 1143 bp, synthetic construct
  source 1..1143: mol_type = other DNA; note = Nucleic acid sequence encoding the B. fragilis S-protein orientation 60-60
aatttcccaactttgcgatttccagagttctcgggtgagtggaaaaagtatttcataagagacattgcagaggtcacgaagggggcgggtatatctaaagaacaacgttcgctttttggaactccttgcattttatatggagagttatatacaacctataaatcagaggtaataaatgatgtccaaagcaaaacagatattgacgcgaaaaatttggttagaagcaaagagaatgatgtaataataccatcttcaggtgaaaccgctattgatatctcaacagccagatgtgtaccttatgacgatgttttgttaggcggagacttaaacatcataaggttatatcaaaatgatgggcgttttctgagttatcaattgaatggtgttcgtaaattagatattgcaagggttgcgcaaggaagttcggttattcacctatatggagaaagtataaaatctctatctgtgtctctacctgcactaaaagagcaacaaaagatagtgtcacttctatccctaatagacgagcgtatcgcaacccaaaacaaaataattgaggagtataaaaaattaaagaatgcacttgcggaattattttttgcaaaatctatcgaatatacttcaattggtgaaatgtgtgatgttgttatggggcagtccccatcctcagtggcatataattacacaaagaatgggcttccattgattcaaggaaacttagatattttcgaaggtgttacttctccaagaatgtggacaagtgacataactaagcaatgtgatataggtgatattatacttacagtgcgagctcctgtcggggatgttgccaagtctaatatgattgcctgtgtgggtagaggggtttgtgctattaaagtcaaagaatctggatgtagcgaatatgtatatcaatatttactgtactttaaagctaaatggggctctattgagcaaggaagcacattttcggcaataagtcgtaatgatattctaaatattaacattccggttattacaaaaaggctgattgttgcaagtcatcttttagctttgtttgacagtgaaatcagtatagaagcattaaacttaaacgtatatacaaagcagaaacagtatttgctaaccaagatgtttatataa

SEQ ID NO: 28 - DNA, 45990 bp, Bacteroides phage
  source 1..45990: mol_type = genomic DNA; note = Bacteriophage Barc2635 GenBank accession MN078104
atggcgaacgaagtaaagggaataaagtatagcgatcttatacagccggacagcagtataaaggacgctattacgcagttggaaggactgcaaaagatatatgacgctatgttaaagcgtatcgaggaaggcgcaaaagggctgcaaaaacctatctccgagggcggaggcgcaacggaggaagggcgtaaaaagatagacgaatacgaaaaacaagtgcgatcattggcaagggcggaagtacagttaaagttagctatgaatgataccgccaaagaaatctccgcattgaaaactaaaatatcggatcaaaaccgattgaataagttgcaagcaaagttaactaacagtatggcgggaagctacaacgcattgtccgcacaatacgagctaaacaaaataaaacttaatgccttatcgcaggaatatttggagaatacggaggcaggaaagaagcttgttaaagagactgcggagatttacgcagcgatggataaataccaaaagagcacgggaaagcacacgttaagcgtgggtaactacaaacaggcgttcgatggtttaggcttttctatatcacaggtcgctcgtgaactcccctccttggcgatcagcgcaaataccttcttccttgctatttccaataacattccgatggttatagacgaaatacagaagttgcgtgcggcaaacgaggcggctgcaaagtcgggggatgaacaggtgaacatcacaaagaaaatgattaagtcgttattttctcttaacactgttatggttctcgttcttacagccttttctatttggggtaaggatattgtagattggatcgggggacttatcacgggtaaaaagaaaacagacggtctgacaagaagtataaaacatatggcggatgccatgcaggacgcacggttagaggctgcaaaggaaactacgaagctgaatatcttgtataaaacggctacaaacaacgctaaatctaccaacgaacggacaaaggcggtgaaggcgttaaagaaagaatatccggagtatttcaaaaacctatcagacgaagaaataaaaacgggtaaagccacaacagcgtataatgaactatctaaagcgatagtaaagaacgcaaaagcacgtgcagcactcgacaaaataacagagttgcagaagcagtttatagatgaagatcaaaagcgtataggtgttcttgttaaaaagacacaagcagagcaggaacttgcggtagcgcaaaagaacgccacacgtgaaacctcgtctttaagtctgttgactgataaaaaggttaatgtagttaacctaaaggcaacaagcaccgttttgaggctgaaaaaggctatcgaggggtacggagaagaagcggagaaatctaagaagaagcaaacggagcttacaaaaagcatggagaatttaaccaagttggttaatgtcgatgcggttacaggtggcaaaggaggtagcggtaaggaagagaaagaaaagtacgacttaacaaagaagtacgaagaaagccggatcgctctaatcattgatgcgagagtaaaggaggaaaacgagataagagaggcggcaagaaaggagttgagcgagctaaagaagaagactaccgagcagcagagagcaactcagatgtacgctgacaccgtgtacaatatcgaagcaaaattgcgtagagacttggaaaagatgcgggaaaggtgggctattgatgatttgcagaaaacacatgacttgcttagcgaacgtttgaacgctgttagacgtgggacgggtgaggaacttatcattcaaacgcagctactcgaaaacgagcgtaaacaggacgaattgcgtatcaagcagtcaaccgatacagagagccggaagaacgagcggttattgctgttagagcgtgcgtatcagctttccaaagcgcaaatgataaaggacttcacggagaatcaagataagcgtattattgaacgctctgtattccgtctcagtcaacaacaacaggcggaaactgccgccttcgatattgtgcaacgttcggagaaagaacaggaacgtttccggttgaaattagagcgtgaaaagtgggagcaaatattagagttaacaaggcagtacggagagcaaatcacgggatacaacgtaaagacggtagaggataccattaagggaatagacaatgcaattaagcgtgatacttccggatgggatagcaaacagggcgtgtttggcaatctgtttgatctcgttttcggtgacgcctttagcgcaaaagatggcaagtcgggcgcagagcgtgcggagcagtttaaagactccattttagaggcttcggagttcgccatagagaacctaaagagtgttgctcaggcaagggtagaggcggcagaagtggcggtacaggcggcagagaaagaagtctcagcccgacaaaaggttttggacgctgagatacaagcgagggcgaacggatacgccaacaacgtagcaaccgcacaaaaagagcttgattttgcacgcaaacaacaggaaaaagcactgagggataagaagaaggcgcagaagcagcaagaacgcatagatacacttatgcaggcaagttctttggtaaccgcaaccgctaacctatggaaagatttaggtttggcagcaattccggctattgcgttgatgtggggatcatttgcttttgctaagataaaagcctcacagctatctaaagcctcgcaggacacggaggaatacggtgacggtacagtagaaatgatcgactacggaggctcacacgcatccggaaacgatgtagatttaggtacgactaaggacggtaagcgtagacgggtagaacgtggtgaatacttcgcagtagtgaacaaacgttcatctcagaagtataagaaactcgttccggacttgattaattcgctaaataaaggtacttttgaacagaaatacttaaacgcctattccggtagcgatgaagttacgaatatcatgcaaggttcaacggttgatttgtctaaggtagagaaagatttgaaatcaatcaaagagcagggacgtgttaagtacatcacaggtgcggacggcacgataattgaagtaaggggaaatattaaacgaataattaaatcataatgaacgttaaagatttgcggtttaaattggggggtgtagaaatacatccccactattcagagctaaaacggaagtttggcaaagagaatcaacaggagtttttcagagagtcgatagaggggagtttaacgctgatcggggcggactaccttcttgttaaaaatgcgagtatcgaggatattttgtacttgcaaatagagcaaaaggataaagggcagctaccaacgcagtatcaagtaatatttgagggctatttcagtaagacagattgtgagatagatagcgataaccggacgtgcaaagtcaagataagcccacgagatgaatatactgatataatgaagggtattgagaacaaatacgatcttatcaagcttgcacccgcattgtctcaaataggggtatccaagcgtccgattgtgcaagtttatattgcgggtgcatctacaatatcgaactaccttgcaggaactcactacgaaactgaggttttcaacgttgtaacggataacaaggagttaacggataagaatttctttgccttcttcgctgcatacaacgaaatcgaagtaaaggcagtaccttatcagttctttaacgggaagtactacggaacgaatggaacgtacactaaattggatggcaatttctcaataaaatggactctaagcgaaggtttaaacattggtttccttcacttggaaaataaagagggaactattttgtaccgatcagataaaatcaattggagcgataaaagttactactacatagacgtttctgaaataacattcacaagaatagtagatgatccgacacttcctcaaaagtttggcggaaacactgttcttttgcaaaagctatttcaaagaatgttgcttaaccttccggagttggacggtaaacctaccgggaaactatcatcagaggacgtttaccctaccaatagtaactacatgtatgctgcaccattaaaggggaactacttttatacgtctacgaaggttcagaacgagccgacagagtacggtgtaaacgatgaaggcaagtattttaccgataacttcgttccggctgtggcgggtactggaaagctgtatccggtatgccgttcacgatgggggaatatgtcgatttggttcgagtttgatttgtcctatgcgccattggaggaaagagcgagaaaggagtatgttttaagggactcgttcgccatacaggacgctattagggcgcttattaagcaaattgacccaacgttaacacacgaagctacggaggaatacagtaagttcttgtatgccgccaataaccctatttccggagcaccttttaaggtgttcatcacacagaaaagcaacatcctaaagggtgagtatgaccgtccggcaaagaaagcggaaacaaccctcagcgatataatgaagatgttgcgtgacacgatgaaactatattggtttatagatggcgataagtttcggatagaacatatttcctattttatgaatggcggaagttataccggtagcggaacggtcggcatagacttaacaaagcttagatatgcaaaatcgggtcagttaatgacgtggaaaactaacacggtcaaatatgataaaaccgatctgccttcacggtttgaattttcttggatggacgatacaacaaacacgtttgcgggctttccgattgacgttaaatcaaactatgtgcaggagggaaagaaagaagaagtaagggtatctaacttttcgtccgatgtggactatatgctattgtccccgggtgacttttcacaagatggttttgctctgttaggagctactcagataggcggcaaatggaaacttccgtttgttacgttcaatttggtagacaagaacaataagaagtacaccgtaaacccccaaaacggctacatgtcgttcttgcacctcgttaaatactacatgcacgatatgccagcctcagagatagagcagggaggcgatcagacgataagagtgagaggaataaaacggagtatgacgcaagatttatctttcacatacgacaccacgcccaaccccgtacaactgataacaacggatgtcggcaacgggaaaccgatctctatgactgaggacttaaccactcgtcaaataaccgtatctttatcttacacccccttatgatagggggtgttttcttttaaattgctatctttgtccctataatcaattttttaatcaaaatggaagtacataacaactttagtcctttggcatttagaaagaaagaatctaaagccacatatgaaaaatggtacgctttcgggaagaattacgctatccccgcaagtgcaaatacgctaacacctttccagtttacagagttgaacatacccgtctttgatcccaatacgattgaggttgaaataatcaatgaagagacgggagaggcgaaaaaatcgggtgtatatgttagctttgatgtaatgcccgaacatggcggtgtattgtacgtatcacccggcaagaactcgtttagggaggctttgccacaggggacgtatagagcacgtttttcaatcggtgatgaagtatatatttcgactcctttttgcgttatacccggcatagaaacgagtagcaaatatctgttgattgagtattggaacgatgaaaagatcgcctatccgggtggcttcattacaacgggtgcgaacaacgacttccggtatcagatgtatgtccctgcaactatctgcaaacctaaatacgagtttgaagaagaattaaccaaacgtgccggatacaagtttttggaactgcaaacgtctacgaaggtgtacgcctttacattcgttgcaccggagtttatttgtgacgctatgcgcctgattcgcctatctgactatatccgaatttcgcacgatggcgaatattacaacgctctcaacttcgagtttgatgttgattggcaggagcaattgtacttggcggctgttgactgccagtttgagacggactcaatcatacaaaaactcccttctttcaatagaagagataaagcgtctttttataatgccctattagcgaacattgatacacctataatgttctctcccgataccgtagggctgtattacaaagagtatcgggaaacagagccagtagtcaagggtaaattgatacgggagttatcccctattgacttgatagatgaaaatacaactattgccgttgatttgggtacaggtgaagcgagaaagtttaacttataccaaatgttgcaggactatatttctaaggcgcatgaagacgcaacggactttttactacatctccgtggaggtgcaacatttggcgatgatatttcaggtagtgctgcgcacataaattcggcaggcgatgcaaaatttaacgatgttgatgcagatgcagtttctgccgattccgttgacgttggttcttggctgtcggtgggtaacactgcatttacggttagcaagtcgggaaagacgaacacaggcgagtttacagcaaggggaaaagcgtatctcacggaagacgtttatacaggtggcggaactggaacgataacgaaagacggacaattaaaatacctttctgcaattatccaacagttcctatcatcccctactttcgtttccggttttcttggcgagggctttaaaatatgggtcgagaatggcaattggcatatagaatgtgacaatttgacagtaagacagactatgaatatatttgatctacttatccaaaagataaggagcgttaacggtgcattggtcgtgtctcagtcgaacggaaagattaaaagcgtgtcggaagatgaaacgaactacgttatcacaatggaggaagaaggggaaacgttccagcctaacgatttagttcggtgtcaagtttggacgggaagcaaaaccaaattctattgggttgaggttgcaagcgtttccggcaactttattactgtgaaaaagtccgaatttacagcaggaaataagcctgaaaaaggtgatgaagtggtacagatgggtaacacgcagaacgcacaaaggcaggctttaatctatatcacagcgcaggaaagcggacacccgtacatagagatattgaacggagttaaaacaaaatcgttatccggtacgaataggacacgtcttggcgatttaagtaacatacaggactctgcgtttccggaaggacaacagccatccggtagcggcttgtattgcgataacgcttttcttcgtggtatattcttgctgagaaacggcaagtcagttgaggatgaagtaaaccaagcgaagcaagatgcagccaacgcagcaacagaggcggagagagcacaacagacagcgcaggaggcgaaagatcggcttaataaatgggctgacgatggctttatatctcctactgaaaagcccgctttgattgatgaaggaaagcgcatacaggcggagtttttgcagataaaaaataacgctgacaaatacggtgtatccatttctgaatataccaaggcttatgaagattatttgaatgaacttagataccattccgcccaacagccggaagatattgcagtgcgtccggaactggcacagagtcagacggcttactacgacaaacgtaacggagcgttgaatgctattgcaacggctactaaatcgtatgtagatgaagcggataagaagctaaaggagtacttagatacggagatcacagcgatacccggtaagatcgaacttgccgtacggagtctgaaaacggcaaattacaacttgctgttagatagtaatcacacgcttagcgcaaacccgtatcaacttggtgtttatagatatgaaacacatttagttgttggcaaatcgtatacgttgacagtgtgttataaatgcgcagattcaagttcaattttagcatataataacgcaggcggggggcatgtggctgagttccctaaatcagataacgaaaccatagtttcagttaaaatatctccattcaatgaagatttaacgtatttcgagttttataagttaccccaaaaggaaacaactcaaacatacgtaaaatgggctgttattactgagggtgatattggcgtgtctaattggataccgtctaaatctgaaatgttctccggaggtagcaatttagtgttaaactcaggtgtttgcgtagataattacgccttgtttggtttaagtaaaacgtattatgaattaaggggtcaagatgttgtaatttcgtttgattatgaatatagtaatcttgttttaggtagcaataatcgtttcggagttgaaacggaagttcctttgtctactggtggtgttcagtatttcggggcttttgtatatgttgactctcgttcaccttcaagcggaaagggtcgatactcttatgccttaaaagtaagtgataacgttgtagataatggtttaaaacacatacaggcgtatgttcaagtaggtagcggtacggtagtgaaaatatgtaatttccaaatcgagaaagggaatacccctaccgagtggaagcccgcaccggaggatatattaaacgactccaaaaagtacaccgacacacagatacttgccgttgacggaaaaatcgaactatccgttaaaactaaggtagaaaatttgggaataggagctaacaacctgtacagctatacttcaactgctttagaacaccttaattcgcctaatatcactatagagagacagctgtctttgcatggtttttatcttgtgggtaaaaatccatcatccgcaggagactcaagcatgagaataatgcacgtaatccctccaataccgggtaagtatactgtatccggctggataaaaggttctcagagtactcctgtaggctttattatagatgtatgtgattctgaaagttatactgttaggtcaacagccgataaccaatggagttattttaaacatacttttgatgtcacaagaaacacagaagcccaaagcgctacatatcactttgtggatttagaatcaatttcatgggctcatatattggtgaaagacttcaaagtagaagcgggtgaaattgcaaccgcatggagtcccaattttcaggatgcagtttacaaaggtgctgaatataccgatagtcaaattagtgtagtcgaaggtaagataacatccaccgttgaaaagataaatactgttgatggacgtgttaccggacttgcgtcacgtgtagaacagaccgaaaaaagtatcacgtctgttgttggtgatattagtgttattaatagtaccaccaataggcatatatcaaagcgaatagatttaagaggatgggacgataataagtttttcccgttggttataagtattccggtttaccacaaaacaagggttgaaataagtaggcctcttaatgcgggatacggaaaaccttcatacggtactcacaatggcggtttttctatgaacttaacgtttgagatgtccggttcgggttggggttcgttgccagcagtaaccaatatctttgactatactaaagcatggacttctgcgggtgcaaagatagttgttgatttgggacaaataactgaaacgtctacgtgtagaatgggtattaggggcggttctatgtatgacgtaacagtagatgatactattgacccaaacgtaatcaacatttatcaaaccgattatcacggttcgcataatacatcgttccccgttcgcaccgatggaactgaacccgtccgcacatacggatactataccgaaataaagcagacgcaggaaagcatagctttaactgcgaacaaagtggatgatcaaggtaggcgattaagtgcggctgagttaactctaagttcagaccacgcaaaattaagcgtagtagaacaaacggcaaattccgccaattccttagcaggaacagcgaacagcaaagccaacacagtagacggtcgtgtcaccgccacccaaaacggcttagtcgaaaccggaatcaacatcacctcccgcaagatcgttttgaagtcagacaacgttctctttcaaaacaatgcaggtcaacagacagccgccatcaatgcaaacggaaaacttactgcaaatgcaattgaagttggtgaggttgttgccggaggctttgcggcacagagaatcactaccggaaacttgactgtgacggatggggcggttatcggtggtatgactattacagggggagtgttgaccgggaagaacatcaatattcaagatggcgcaaagatcggtaacttcaccattgtatcgggtatattttccgcccaaaatacgcccgcaggcatacaaatgactctatctaataatgcagctacttttgacagtagcggagtacgtgtagaacataattctggtggttatgcgttgactactacgggtaacggaagaatattcctaacagggtcaaatttttgggttcagtgcaaggatgttgatttcatgggtgcgcaaacttggaaagcgcccggggtgttttacgcatgtacaatattgggtagtggtgttatcggtgcaacatggggaaaccccgactttcatataacgagggtggttttaaactcaaccggaagatatactgtgtatacaagtggggggcaagttgggaattactttgttatggtgcaaggttatgatccatcactttggttaagtactaccgtagagccttattcagccggacaatttacatacaaagtatttgatgtgaatactggactgaaaaacgcatctgttatcatatatttttgcggtatggttagttagtttaatagttaacatttgcggtaagttggtttgtaccttcttaccgcttacctttgtaccaaacattaatcaattaatataaaattatggaaaagaaaagtttagattttgatttaaagtcagtagtttacacgaaagagacaaaagtgatggactaccattttgagacggaaaacggtaagtacgtaggtcaattaaccacggtatcgacagagccggataagtacaacatcactcactgtacggctgatgtatctgaaaaacagatggtagaaatgccgggtacttccggtagtcccatcttgcaagaacaatatgttccggtcggatcgcttgccatccgtgacggtcgctttgaggcaaaccagtttcctctatctactaaaacgtccgtctatgtgaacgactttcaaaacttcatctttgcgttaaccgcacctaaaacagtagaataatgaatgttacacaagaacagttaaggttaatgcttgtatcggttataagtccgatacttgcgtttcttacccctacgagcggttttataaccgcacttgtgttcatgttctgctttaacattatttgcggtctgcgtgccgatggggttaatttgtcggtgaatggcgtgcgtaggttttctatgctaaaattcatctcagccgtgcaggagcttattttgtacatccttgtgatagccgttatcttttcgtccgtggcagagatgggggatcatgatgaagctattttatcggcaaagacagttacatacgtctttatgtacgtatacctatcgaacgggtttaagaacctttgtattagttacccggataacaaatctttccggctgatataccacattatccggtttgagtttaagaggctgatgggcgaacgtgccgcaaagatagtcgaggaacacgaagaaaagattgagattgaaactaagtaattaacacgggaggtttaacgcctccctttaaactttatcaaaatgaagtatttcacattaaaagagctaacacgcgcaacaacagcaacggcaaaaggcattgataatacgccaacaccggaagttgaaaagaatttgaccttattagtagaaaacgtattagaccctctacgggagatttacggcaaaccgatcacggttaattcgggctatcggtgtccggagttaaataaagccgtcggaggctctaaaacatccgatcacgtgaaaggttttgcggctgatattaccggaggcagtaaggaagagaatgaacgcattttcaacataattaagcacaatttccatttcaaacagttgatagatgaaaagggcttttcatgggtgcatgtctcctacgatccctctaatctcaaaaaccaaatactaaagctatgaaaaagcaattatttgagtttttagcggcttttgttctttgccttggcattgtgtcgctattactgataaacgctgatttacggaagaaaaaggctattgcagaaagaaatgttagcatcctaacaactcagaacgttgcgtaccggacgaaaggtgggcaaagtgccatgaaagtagaggaattgaatctgactttaaagcagtaccggaacaccatacaagggaaggataacactataaaggagctaaagcagtctattaaggacttgaaaagtcacacaagcgttcaaacatcaactgagacgcattttagcacgccagtacgggatagtgttgttcttcgtgatagtttggttatcgacacaatgaaatgcgtaaatatgcgctctaaatggcttgatatatccggttgcatagatagcaacggcacgtttgccggaacaaccgtttcccgtgacagcttggatatacttaatatagagcatagaaagcggtttttgtggtttcgattaaagaaggtgaagtatagagagtttatcgtaacaagcaaaaacccctatacggagataacaggttttaacgtaactacgatcataaagtgataattccacgttaaaacggttaatgcacgttaaagtatttgccactgagaaatatatccgtatatttgcagcgtagaagttattactaacgtcactaacagcggttattggctttcatatttatgcttatttaaatattttgtatcacattctcgaactgtcggtatgcgaatatagacagtttttaattagaacattttcactaactatatatattgggttttgtcataattacatttttccccctccgcttgtgaaagtagagggtttttttattaccttatccgaacacgcctaaaaaagttaaattcgtgttaaatattaaacttatgctttgatatttaaaatatctccttaactttgcaacatcaaaaggaaacgaattactaacaataaaacttagaattatggaagaaaaggaatttatttattgcttgacaggagagattaacgtattaggcactgtcaaggctaagacaataaaaagtgctatgaaacttgtagcggctattcagagaggtgctatattgaatgatccggaaagaaaatcaatcttttggagtgtttcacgtgctgatcgtccgtttaagttaggtaatattgtgtatacaatatgctatcccgatgggtctatacgttcacatgtatgctaacaataaaaatttagaattatggaattagtaaaattcagagaggcaaagaggataatggaagaaaaagcctttttgcaaaaaacgcttgaaaggtttcagtccggctatcttagtagaacagatttatatttcagttcgggaagtagcgtaacattttcagaaggggatggcgaattttatgaaggtttgtgcaaagacttagaaaagtctataagagaatctattgaaaaacgtattgggaatcttgaatctaaatttgataaactatgatacgatcatttagtaagtcgggttcaacttctatgctgacagataaggaaaaagcgtttaaccgctactgcctaactaacaaggaagtttcatacaacttaatgcgtatagaaatggcagttgttcaaatgtcgtattacggcaaccgttcatcggacgttacgttaacaaccgatagttctgaggttttggatgcaatttatacagtcctaacaaacgaagggtttaaatactctttcaatttacctaataaagtattaaccataagtattttttaatttaaaatttaatcaaaatgaaagaagaagtaaaattgttcagagcgttaattattgtttttgtgttgcttgtgttcaccttcgtgttaacttcatgcagtgatgatagcgacaatgtgtatcaaacagaatattctattgatgttccggagtggcaaactgtttatgtgaatggtgaggttacaacatctatatcaccatacgtttgggaacatgtggacttatcagataaatgtgttagagtattctccgcagggcatgttagttatcataaggttacaatggtgtcacgtgatgatttaggctttaccgtttattcaatagaaagtagcaataacgaacggtttgcatacaataagaataaaggtatattgcaatattggtgcacaagaaatggcattgaaaccgttgttgtttatcgtgaattaaagtaagtttcattttaccctcacccggtggcggttaaccgggttattaagtatgaaagtaattgttagatttaaagaagaagaagaaattaccaattttgaagctaaatacggtttagaggtagagaaccataacggacaaagatatatcattgagttcgataatatgggaaatttggtggttaatagtccgaacggtgcgttattggtaaaaccacaatgcagtaataaaatatcaattagacccgaataacatgaaagagataaacgaaactcaattacagctatcgactgagggaaaaagacttcccgatatgataaagcaggcgaacggcatacacgaacttgttaagcagaaactttctgagtataactcaatagagtataccgatgataatataaaggtggcaaaagccgatagagccactttaaacaaggcgaaaaagggacttaacgacagccgtatagaactggaaaaggcttggatgaaaccattcaacgaactaaaggatgttgttaacgaaacttgtaagctgatcggtgaagcttcttcacgaatagatagtaagataaaagaaacggaggaaaaggagaagcaaaagaaactggatcaaataagggagtatttcgaggaacacaatgaaaatcttatcttgtttgattttgctttccgtccggagtggcttaataagactaaagcactttcagttgtgaaaatggagatagacgaattgttcaaaacagtagacgatgatcttaacagactgaaagagcattttgcgggagaggcgttttatattccggttatcgacaaatatacgtctacactcgattataacaagtcgttcgactatggaaaccacctaaaagaagctgcaatacaagccgcaaacagacagtttgaacagaaggcgacagataacacgcctcagcaacaaaagcccgaaattaagccccaaaacgagccaaagacgaacgaagaagaagtttatatacgaggctttaaagtccatgtaacgagaaagcaggcttttgcgcttgctgagtttatgaatagccacaatataaagtttgaaagtatatcaatatagacggtagcccaattgggctacctttttgttttgtttgcaatggttaatctattgttaaaacttaaagtttcaattgaactttcaaataatgttcttatatttgcagtgttgaaagaaacaaagtagtaacaattaaaaattagaattatggagaaggttagattaacaaaaaagcaaaaaggggataagtttgtttatgcagttgtagacgaaaagaataacgtaatttctacccgtacatcaaaaagagattatgtagcgtgtacagttaatggagagttttatttcggtagaattgatttaataggaaagggaaagcatggtgaatgtatttcatattatactaatattataaatgatccggaaaaggaatacataaagtttattaatggattcgtaccttcatatcgtatagaaatgatgaaatacaaccctaaaaatgaatggatagataggctattgaaatgtgcaaaagaaacacttcctttgttgaatacaatagcgtatttgtctaagtaatattaacagggtggtagaaataccacccgaaaaacaaaaatatttattattatgaatgaaattaaatgcgttaaatgcggatgtgtgattaatacagatgcaggatactatgctgcttttgatggtggcttttgcagaaagtgttggattaaacaatcagataaatttaagagtgaacagttggtgaaagcactcgaaaagagattcaaatgtaatattaacaaaaatttattttaaaatggaacagtatttagacttactaaaagagactttaaattatggtgaaaagagatcagaccgaaccggaacgggaactatcagtttgttcggtttacaacgatcttatgatttgcgtgacggtttcccacttgtcacaactaagaaggtattcacgaagggaattatacatgaactcctttggatgttgaaaggtgacaccaatataaaatacctaaatgaaaatggcgttcatatttgggacgattgggcaaagccttccggtgatcttggacgtatatacggtaaacaatggcgtgactggcgtataaatagcaagttaagagtagatcaaattgagtcagttatatatatgattaagtttaacccggagtcaagaaggctaattgttagtgcttggaatgttggagaattgcacatgatggcacttcctccgtgccattgctttttccagttctatgtatcagattccggatatttggacttgaagctgtatcaaagaagcgcagacctatttttaggcgttcctttcaatattgcgtcttattctatcttgctgtctatggtagctcaggtttgcggcttaaagcctcgtagattcattcatacgatcggggacggacacatatatttgaatcacgttgaacaggtgaaagaacaattaagtagagaaccgttctcccttcccaaattggagctaaacccaaatgttcgtaatatattcgactttaagtatgaagatattaaaatagtagattataactgccatccggctataaagggggaggttgctgtatgaatgacgaagaacttttcaagtttttatgccgtaatagaatgatctattatgaaagataccttcacatggaatctgtatattatctgaataacttgctaaagaaaacatatagttcgtttttgaggaataggatactgaacgctataaatcataaattagctggcttataatttaaaagggatgtgcaacgctttgccatccctttttagtttctatatatcacataccgaaactatcgttgctctatgaaacaaatctaacaatgtgtagtaacaagtatgaaagtgatacaaaggtaggcttttgattctatctaatagttaaaacgattcgttttacatttcattaacaataaaattaaagaatttctttgcgtatttaaagtttatccttaactttgcaacatcaaaaaagaagtagtaacattaaaaacgaataatatgcagattaaaaaagatcgaatttacaaattgcttgcgcaagtttgcaagaatgaaggtatttcattctcctacgaaaaacttgttttatctcttaataagtacattgatgaagacgaagaagattcagtattcgggtataaaatatctgatattgatatttcaatcgccaaacatatatcagttgatatttgcggaacgcttgcattaagcaatattatttgccaattaacttgcatcggtgcgggagattgcccgaattgcggaggtttactcagattgatagaatcttatcccaaatttagcaaacagtattgcgatcgtgattgcgagccggagagagaggaagaaaatgtatatgaatgtttaacatgtggaaaggaggttattttatgaatattgaaaacacaatgatccgtatcaatgatgcgattataagcgcacgtatgaacggcaaaaagattacgaaaaaggatattgcagcgttgttgtggaaggactcaaagcaaagaacgcaggctgtaaacatgtccgccttgtgtaaccacaaaacccaaactataaaaatagagtgggtgaaagagatatgcgaggctaccggagttgatgcgaatttcttgtttaatattaaccctaaaaaataaaagttatgattaaaaatttgcccaacattcaaaacgaaatgaatgttcaaaagtcgaggtataaccagtttggaaaatacaattatcgttcgtgcgaggatattttgcaagaagcgaaaagagtgtgcggaaaatacggatgttatgttatggtgactgactctatcgaatttatcgaagggcgtttttacgtgaaggcaaccgcaaagattgttgagactgaaaccgggtctattgaaacatgttcggcttttgcacgtgaagaagatagcaaaaaggggatggacttagcacaattaaccggtgctacatccagttatgcacgaaaatacgccttatgtgggctttttgcgatagacgatagtatagacagtgattcaattaacggagagccggagacgaaagaaaaacggcaaaagacatcctcaaaacaagctaccaacctaagtaataccggaagcaactcaaattatttgggcgtgctgcttgaagaaataaagaaagcaacaacctataaacatttgggtgatattcataagaataacgctaattaccatcaaaacagtgagtttatgaacgctttagttgttcggaagtcggaacttgaaaaagcggaagcagaagcaaagaaagtataaataatttgggggatgcgttcccccaataaaaataaaagcaatatgaaagaattaacattactccccaaattggttaatgctgatgtaacgtatatcagcgaaacacatgaatatttttcaagcgattttagaaagctgagaggaataacaggttttatcaacgatcaattatttcccggaaaacttgataatataccggataatattttgagatcggctactgagagagggaaagcggttcacgatgaaatagagagaatcgacaaagaaggaattgagccggaaacggtttacggagagaactatttgaatttaaaagccgaaagcggtttaattcatatcgcatctgagtatattctaactgataacgagtttatcgcctcaccgaccgataaggtttatttgggtggttctgataaatcggttgtattgggagacgttaaaaccacctataaacttgatttgctttatctgtcttggcagctatcaatatacgcctaccttttcgagaaacaaaacccaaacttgaaagtagagggacttatcgcaatttggctgagaggtgacaaggataaggacggcattttctccgttgaacgcataccggacagcgaaatagaattgttccttaattgctgtaagaatggcgttcgatatgcagataatgcaagtaaagacagctacatagcaaaattggagtcactgcccgcaaaagttgcgcatatcgaagaaggcgtttatgaacttcttgaaatgcaaaaaaagatagacgagcatttaggcaagtttaaagaacagttgttaggtctgatgtctgaggcgaaagccgacaatataaaaggggagcttatttcagtcacaagaaagaaagcgtatagccgtgaatcacttgattctaaagcactgaaagagcaataccccgaaatatacgatcagttcgttaaaacatcaaatgtcaaagaatcaattcaattaaaagcgttgtaataactaaacagaaaggacaaaaaaatgaaaactggatttaccaaaaaagaagaaagcattatacagcaaatttgtttgttatataatatacagataaggacttataaacagggtgtgttcataggaatgattcctaaaaacgtccgtatgaccttgaacggtacttatatgatggaattattaaataccggaagcgttgtttatcttgaaataaaagatggtattaatgtactaataattatgcaccaataaaaaatgaaggaaattaaagaatcagaattaaataccctcttagaaaatggtgatctttgtgattattgtccgtatgttagaggcgaaatatctaaagggctttatgatttatgcgaagggtgttattgcgaacaggcaaaggataattatgtacttgaaaacgatttagattatgaagaatagatttatgaaaaggcttattggttattcgcattatgtggataaacttgtatatgtaacggtagacgggggaagaagggaaataatgaaattaaagccaaggaaaaagaagttttataattccctcgaatattacaaggctatacaaaagatgtttgaaggatgataacaatatctgaaagtttagcgaacgagatcggtttagagggtgcaactgtgtacagttatgtatccgtaattctatcaacggacttttataaggatcgttttaagggatgccgggttaaaggcaagaagtatacagccttcatatcaataagcaagttaaaagagataattcccttcctatctacaaagaagctgtacaacgctatgaatttgttagtagatacaggatacctaaaagagctaccattacgaaagccgggtttaaatacaacccgatgttaccaacttgttaatgtgatccaacatttcgagtaaccgataatacacccaccaacgtttttttagttgtgtgggtgttttttgtacagcacgattctgtgcagtatgtctatacctaattatcagatatttgcaaaacaaagaatgttaaatcataaaaaaaatagatgttttttcattgcagtatataataataaacactatatttgcagagtaaaatttaaaacagtatgtatatgaaagtagaaaacttagttaagattaagagttatgccgatttaaagggagttacagtaccttggatatggaaacttattaagagaggaaaattagagtatattcaaattgatggtgcatgcttcattgagttaacggatgaagaactgaaaaagtatgcggagtacaaagaacggataagttcatttttgaatagcaaatagtattaatcataaaattttagaaaatgaaagatttagtttttaagggagaatcaaatcaagttttaacaagcagcttattggtagctgataagttcggtaaaaaccacaagcatgtattagacgctataagagagttaattagtagcgccgaaaaatcggctgtactaaaaatgtttgttccatctacttatatagcatctaacaataaagaaaacccgatgttcataatgaatagagacggttttacgttgttggcgatgggctttaccggagaaaaggcgctgcaatttaaattggagtacattaacgcctttaataaaatggaggaagctattaagaacggaggttttaacgtgcctaaatcgttccgtgaagcattattgcttgctgccgaacagcaagaagttatagagaatcagcaaaagcagatcgaagaaaaaaacgcaaagatcgaagctgataagccgaaggttctgtttagtgaagcagtctccgcctcgaacaaatctatcttagtgcgtgaacttgcaaaacttatcacccaaaacggttatcagatcggggaaaagcagctatacgagcgactgagaaaagccggatacctttgcagcgttggagaatcacgcaatcaacctacacagacatacatgaatatcggtttgtttgagattaagaagcgtgttattatggacggtgatgaagcaaaggtttacaatacaactgtcgtgacgccaaaaggagtacattatttcattaataagtttttagggaagggaatgaaatgacgcattgttttgacgataaagtagcaacaaagttaggagttgaagcggcatgcgtattgcacaacttcgctttttggataaacaagaatatagccgataaccataattattttgagggcagatattggacttataacacaagggaagcgttatctaaacttttcccgtatatgagtcaatctaagatatacagagtgataggaaagttggaggaagaaggctatttgttgaagggtaactttaataaatcgggtatagatagaacaacgtggtacgcattaacagacaagtgcataaagttcctttttgagtgcggatatacgcttataggctattctgagccgattttgcaaaattgcaaaatgcaagttgcagaaatgaacaatgcaagttgcagaaatgaacaaacaataccagatagtatatatacagatagttatactaaatctcctaccggagattatagtatagccacgcacgaagaatctgttttgttcccgattgaaaagaaacctttagcctcagagatattcggctttaccgcaaaagccttagatgtaacaaagaaggtgatagagcgaacagatagttttttcgatcagctaacattcccgtttgagtcggaggaatttaaaaaagccttttatgtgctaatgactcaaccaaagtggcgggtaaagactaagactctaacagctatgcaagcaaacctaaacgagattgcgcaatttgaagaaggttttgctatgctattgataaatcagagcatatctaagggatgggcttcactggtatacgagtcaacgccaaaacagtatatgcaatggctacgggaaaagacgggagtctccggaaatacacagcctgcaaacaatactaaatcgtattttcagagtgacgaacagcgcaggatgtatcagtcttatttaacggaggactttatatagcattttaaggcttaaatttcaattttaatcactaagacaataaaagtatcatgtatttggagaaaatcgaaaattcgggcggaaaattagcaaaatacgaaggttgcggatcgtttatagagaagaaccgaaaattttatgaaagtggcaacttcggacagctatcaaaagtagatcaaaagatattccgtgattcaactttgcttttggtgtccgaatgtacagacgaaagaaaaagaatagataatttttctaaggttcttaacggagtatgtttagagactggtttaaaaatgccggatgtccgggacgcaggaagtatattttatgctgtttgtgatgtgatagatatgtatttcgatgatctatcgttcaatgaaattcgtttggcatggcggttacttgctgtcggggaactcgacccgtttttgccaaaagacagatacggtagtccggacaaaaatcactatggctctctttcggttgattatatttcaaaggttctaaaggcgtataagaaacgaaaggttgaaacgatggaacgagtttctcagattatgccggacgaaaagccaaagccgacacctgaacaggaaaagatgtttttaaatttgcaggcatacaattttattctcgcccttttgaagtataagtattcgggacgtttccacatagagcgtgacaggataataaacgagtctacatttgcgtacatggaacgattgggatatgatatgtcggtagtacctacgttagcagataagaaagaagctttgtttcaatttcaaggtagaccagtaaatagctttgcgcaaattttcgaaaaagagtgtatttcgaggtttgggatagaccacgaagcagtttattttcgtgcggtactgatagccaagaaaagaaagttattccagtattgggatgaaatgttagccttatcaaatgaaggtgatagatcagaagataatatttggaagttgtattactacattcaataaaacgaaaatcatgaataaaagaaaagtaaaaaaagaaacttatcggataaggcttacaaagccttccgataaattcgtttacgtctctgaatcgttgatatatgaaaggagaaaaaaagagggaaagagatgttatactgtatatgtcaaatatatgaatgttaaatatttgtgtgtttcccgcaaacaggcaaagaatttaataaaggggttcatgttaatatgggaatagatattatttgcgcaattgatccgggtgtgtcggctggtgggatagtggtatataagccgggtaatgatcttgttactattcctatgccacgcacggcaaagggtatttttaacgtgtttcaaaaagtgaagcgttccggtagtcctgcaatattcattgaacgtctttcggttcgtgggggtgactccggaggcgggaaagaatttagaatagcaactatgttggagaattataactaccttgtatgttgtgcgctcgttcttgatattcctttgttcctatgtgcgcctatttcgtggcaaagtggtttaaatctgagggagaaaggagagaaagaggaaaagaaggatagaaaagaaaagtatctgaattatgcaatgaagcaattcccacttgcaaacgtgaaattatggaatagtgacgctatatgtattttgcgcttcgcacagatgaaaatgatttgtgatatagattggttttcaagtaacatgcagaacgaaaacagcacggaaatatcattttcttcccctctgttggacgatagtattaaattcgtggaaagatatgctttcaaaagaaaacgagctaaaaaacgatctaattgagtcggtgaaagagttaagaagcgcacagaagcgatttgaacgattcggtgagagatacagagagaggaaagaaaaggcggaaaagaaagtagatgaaattctgtcggttatcgaagataagcaactatctattttctaacaaaagttaaataacgggtatttcggaaagatttacccgtttttatttgcgtgaatttaaagttttgctttaatttgcagcgtagaaataaaaacagtagtaacaataaaatcaattaattatgcaggaaattaacaagaaattaagtgaacaatcagtagaaaaggttttggatagaccggagtatagaaaagagctttctatttattgggagggcttaaaagagcaacgggaaaaggtatctttccaaatattgaggaatggcggcattcctaaaaggataacaatagacagagttgggagaatggatgcagaccaacttgtatcagaatttaaactgatacttgacagaaagagtgagttgcctgcaagtctgaggtactttatttcggatgtatgcggaaaggtatttatcagttggtttacaaaagtgatcgaagatgaagcaaaagaaaataacgatacccgggaaggtaactaaagacggtaagttatccatctacatgggagagcttaacgagtttatgaagaacaacgcagggaaaaatgttattgcggagtttacagtattagaacggtctgattcttcatccttgcgtggatattatttcaagtacgtagttccacaatttcagaaagggatgtatgaaaatgggtacaggtggagtgaagaacagacagaatcttatatgcgtagtatttgccctattacgatgggtgaagttgtagacgttgaaactggtgagtatagaaaggactcagttaaagttaccgatttaagcaatagcgaatttgtcgaatacatagactttttaaagcagtttgcggcagaagaacttagtatttatattgaagaaccaaatagatttgtaagatgaaagaaaatgaagaaatgactttagaggaaaagttcaatttgatgtgcgaggcattaagtatatcaccggagagaattatagatagggatattacccgttatgtatcacttcgcagaaattgcattatccatcagctttacgcctacaaaaatcacggtttacccgaattgataggtcgcacgaaggttttaattatgaaagcgcatgaacgttttcaaggcgaattagatgtgaaagatatgacagccgtagagtttgtccggcttatagacgaacgactgcaaaagtatattgatggcaaagaagattaagaattttattctcgttcattgcacggaatgtaaattcagttcagatcaccataatttgatttgctattgcaaaaagagagataaaaagttatgcagttgtccgaacattgggcgggtatgtgagttttacattaaaaaataaagtatcatgttaaaagacaattttgaattaaagagagttaagttcttgaataacggtttagaagttgattacaatgattgccgtttgattgatggtgaagaaacaaagacgtttcacaaagtaaaatgtcccgaatacccacatagagatttaggaatttcggcaaatcagcttcgttcatatatcgttgatttgatgggaataatgaattttaagaacatcacctatttgtctgatttggcaaaacaagacaatgagttaagtagacaattcgatgaatattttgaaacgcttgctacccgtgtcgccattagcgagatagtttacgatcccgaaaagaatacaatcgttttcaaatatattttcgcaggcgtagatttatcccggttgaaaatgcaaacgagcaaaattatgttggacggtgaagggttgaaatttgaaatagcactacaagaagattttgaagcactgaaagatgaaattttcaagtatctttttgagaataagcgtgcacaattggagctattcggtgagacagcaacggcagaaccggacgatagtttgacgcccgatgatgatttagaaggtgacgatacgttttttgatgatgaagaagcagagcagccggagttgatcgaagaagatgtacacgattgatacgtttgaggaaatagattattgtttaagcagggggtataaccccttgctatttaataattatttcgatattgaacctaaaacaaggtatgaatatttaaaacggatgttcggggagggtcacggacagagggaaaatgaacgtttcttccggtatatgtgggatattaagcctcactattgtgaagagtgcttaaagccgctaacagggtactcagccgtttatatttcacatattgttacgagaggatcgaacccaatgattgcgcatgatcctcgtaatataaacatactttgtttcaactgccacaatcgttgggaacacgctaatacccgcaaggggatgcggatataccaaagtaatttagaaaaaataaaagtcctcaaaagggacagtttaaaactgcaaaagaaatgaaattagtaaaatttgtaaataggtcgggaaataaatttatgattaaccctaaatcggttgaatcaatagttcaacatgaagatgattgggtgtgtattaacgtaataggttgcgatactccgtatatagttaaaggttcaattgaagatgttaacaaagcactaaacgaaagtagcaagattgattcaatagccggacttatggttatcgtctttattggaatttacatattatcaacattagcaaatttattatcgtaatgaacttaaacaaaatcgaattgatcgggcgtgtttgtgctgatccgcaagttaaaaccttcgataacggaggaaaagtgtgtaacctttctattgcaacaaacgaaagggcatataaaacgagtaacgggatcgaagttccggaaaaaacagactttcataatgtaacattcaaaggtaaattggctgagatttgcggacagtatgttaccaaaggaatggagttatacgtagagggtagtttacactatcgtaaatacaccgactctaataacgttgaaagaactatttctgagatcgttgtaaggtctatgcagatgggaagaaaagcgggtgagggaaaccagccgccagccggaggcaacggaaaccaacagccgccaaccggaggttatagcggtcaacagcagccgcctcagcagatgtttacgcaaaatgatgatttgccgttttaaggtagtttctaaattggggatgtatattgcatcccctttttgtgttaaatacatgttaaaacttaaactttagattgcaatattaaatattatccttatatttgcaatgtcaaaaggaaacaaattactaacatttaaaaataaatattatggcaacaatgacatcaaaacaattttgtgagagaatgtatggaatgtatcacttacttggcggtggtgatttcggatgtgctcactgttcagacaataggttttcttgcggatatagaaaggagaatacagttttaaccaatgcacttatgaaggcgtgtgataatcacaaagttccttataagatcgaggcaaacgaatattgtattaatttcgtagtagaatttaaataataatagcggtagaaataccgctttaaacttatagttatggaaagaagaagattatccggtcaatacaaaatagcgatgttaaaaaatagaggaaacgatacattcgcgggaactgttgggataagaattggtttcatgtatcaatgtggtgcatatcagtattttactttttgggagaatgacaataaaatatcggttacagaatcaagtaccggttttcgtgtaatgtctttggatgttgaaaagggtgagactcctaaaattacgcatgataggatagttgagaagttgaaaggttttgaaccttctttgggagactggaatagtgctaaagaggtgatgaagaaatataatattccctatcctcttaatgaatggatcgtaggactaaaagacataaaccatgaatgaagaaatagagaaagcaagatcggtgagtaacgaggttatttcggaaactataagaaaatcgactgagaatataaaggcaatggaggacgatttcagattagtaagaaagaagcttcggaaaattggcgatcgaataaaatttgagagaaagaaacttgatatatacaacgaagaaataaaaaggagggttaagtatggaatttggtaacttactgttagatagattggggttcgaccgtgaaatgttggaagctaaactttcggaaatatccgctaaggagaaagagataagagttctaaagaaagaagtttccggtataatggaacacatatcaaaattggaaagtgcgttaaatcatggagagcattattattgcggtgcttgctgctatcttgaaagtaaatgtaataagggaaaatataagtgtcttgaaaccggagaatacaagaaatatcactgtaaagcgtgtgagaaatttagagatttaccattttaataactaattataaattaaatattatgattgattttaatcaaaaaagtatctctttaactaaagagtgtacagaacaacatgaaagaatgaaggcaaaaggtttttatgactcagaggtttttgagtgtaaaaaatgggcgttgatagtgtctgagttctgcgaagctatggaggctgaaaggaaaggcagagttatagaaaacgatgtatatgactttgctctgaataggaaatcaaagataggctttgagtcgtatttcaagaaatggataaaggatacagttagcgatgaactcgcagacgtgttcatccggtgtatggacgcaataggacattctattgataaaattgcgtgtccttccgaaatttttgttttccaaagtatggttagcgatcatttcaataggttattgtattttgaaaaatctatttcatcaattgtttattatgccattcaatttgtaccgaaatctttatttggcaaatcgtgcattatcgagtatactaacatgatggcaataaccattgcagccgcaaaactttataacatagacctatctaaagcaatagaggcaaagataagatataacgagttgagaggtcaaaaacatgggaaacaatattaattcattatggaagaaaaaattattgatttagcaagaagaagcgtttattatggtgatccggaaggttaccaagttggggggtgccattacaagacatccggaatgcaactttctgagtttttagaaagtaataaagttggtttcttggaggggaacgcaatgaaatatgtgtttaggcacgataagaagaacaaagaagaagatttgctaaaggctattcagtatatcaaattgattctaaaatacaaatatggtaaatacttagtaggtgatattctgttgagtgaagaggaatatagaaaactggatgagcttatcgagaaacaaaatacgattgaacttgatactacttttatcagaaatgcgttaaaaaccacatcaatcagcgcaaacaaaatatcgggagaaaaagcaaacttgtatgttgcaaagctaagagaggttaaagccgaatatatcgaaagttttgttttgtcggacacaaaaaaacgcaagcttttcgatatgggactacggtatagtgcgactggtggtatctatattcgctttgattctaaaaacggagaaacaatatgtgttaagccgggtttttatattgttctaaatgaaaatgggagatatgaatcatactcaaaagaaaagtttgagtctatttttcaaccaaaatactaacaaaaacaaataatgataggtcacgttgtaaatatagcagcgtgactttattttatattggctataatagtgttatttttgcgcatattgaaagattatataatttgtagtacaatataccgaatagaaattataacttaaaaatacgtcttaaaatggataaaaaaataggttcaatgaaaagagggcagggaaggcacagccggacggacgaacagacagaaagagatcgttcctttgcctctgatttgtttttgaaaggttattcttatagaagaatagcggaagcgattaacgagcgaaataaggcggacgaagtgccgtataccgtgacttatcaaacagtgtataatgatattcagttttgcttaactcagtggaaaagagaacagttcgataatatagatcagtatattacgcaggaacttcaatctttggataatgtagcccgtgaagcgtgggaagagtgggaaaagtcaaagcgtcccaaatgtaagacaaagtatattttaggtaaggcaaaggaggttcagaaggaaacgacaacgggtgatccttctttcttgaatgtagttctcaacgtacagcaaagaaaagcaaggttattgggatatgattctccgctatgtataaacttggtaggagacaaagaaaaggaaaagcctaaatacgatttttcggatgttccggaggacgttttagaacaattggcagattctttgcaaaatacggagggtaaaaagtgaaaaaagtaaatgaaataccaccagttgagattgtgaagcatgttgcgaggaagaagtttaagaactatgcaaaattcatagatgataaaatagttctgagtcagtttcacaaaacatactacgagattctcgataggtttgcacatggtaagatcaaaaaattgattgttaccgttccgcctcaaactggaaaatcagagggtagtagtagaaagctaccttctttccttttggggcttaacccgtctttaaagatattgatcggttcttatgccgcatctctcgcagaggggtttaataaggatgtacaaagaattatggatacaccggagtataaaagcctattccccgacacccgaataatgggagaggaaaaaaaaacgaggtatcaagcgtttgcgaggaattcaaaaatgacggaaacaatcgggaaaggtgggtatattatatccgttggtcgtaatggtagtttgactggtaaatctgttgatatagccattttggacgacttatacaaggaccatatggaggcaaattctccgattatccgggaagctgcttggaaatggtacaccaccgttgtaaccacccgtctacacaataacagtcaacaacttattgtgtttacgagatggcacaaggacgatttaataggaaggatcgaagataaagagaatgttatcaatgttgaaaagtgggaagatttggatagtataccggaaggtgcgtgggttaaagtaaactttcccgctttaaaggtgggagaaccaacagagattgacccacgtttgccgggtgaagcgctttgggaagaaaaacatagcgctaagaaattgaacgcacaaagggaacttgatagaaatgaatttgaatgtttgaaccaaggaaaccccggtagtgctgagggaactttatacggtaactttaaaacgtacaccgataaaaacgattttggtgtgttggtcggaaggggtaactatacggactgtgcagatacgggtagtgactacctttgttcaatttgctatgataagtatcaatcaaaagaagcggtttggaatgaaaaggaaaggaggtataagcatcttattttctgccttgtgacggacgttatttatacgactgagccaatagaggtcacgcaagtaagtgttcccgatatgctaaatagaaatgatacagattatgcaaatatagaaagcaataacggaggacgctctttcgctgttaatataagccctaaaacaaagactgaaataaattggttctgtcagaagttaaataaagaggctcgtatattgtcgaacgctgcaaacgttactcagtctattgtaatgccgtacgggtgggagtcacgtttcccgaaattccacgaacatataacaaattaccttcgtgaattttccgccaataaacatgatgatggtgcggacgttttaactggcatagtcgagaaagaagttattccaactatatatcaaaaaagaagaggaataagggttataaactgataaagtaggaaaatgtatcagactttcaagtttatacggtatatttgcaaagtaaaatcaattgtttaactaaatttttataattatgttgtattgtgattgtcctttaggagcagcacttccggatattcccgcatttagctgtcccgacaatttcgggcaagttcaaaaacttgcttttcagagactcgaaaaaacggcaggaactgcaaatactatgactgcagaaagtatcgcaaagttggctacatggactcccctactgtcagcaaaagacggtactaaagtagtagttacgccttatatttacgagccgacagtagaggcgggcgctgcccttacttatggaggtggaaacgcaactcccggaggtattgtagatattttagggtcggagtcgacaccgtttacagcttcgttcaagaagttgccgcaaaccattatcaaagctatgaaagcgttgatgtgtgaagcgggtcaaatcggtgttttccttatcaacggtaacggacaaattgcttgtgataagacgggtgagaatttgcacggtttccctgtttggtcgctgtttatcggtgataagactatcggaggtttggaagctccggatagcaatgctattacgtggaacttcatgcctaattggtcggacaacttcactatcgtgaaacctgagtttaaccctctgactcagttagtgccttctgcgggagtaggcggatgatagctaaaaaaacgtatatttccctcagttgtgaagaactgggggaaactcgtttattcgatattgaacacgctgagagacttttgggaatggttaataatggagggtggcatataccggaggactcagaatttaaattaaatgaaaatgggaaaatcattagacgaaataagggagatatacagacatccggaggggataagtcagatagcgaaagcgaaggaacacgaagaaagaatagcgtttcacacacgggtaagaacgagcgatgatcgcaataagccagtaattgactttctttctaaggttaagacgtggatagcgaaagacaaatatgatattttcctatctatgttccatttcccggttaaaacaaatggtgttacttctgagatattcgacaaactgagccgtgttttcgatggtaggaatccggtttataactatcagtttaaatcatcagaggatcgtgacgactgggagtattaccggacggatgttttaaaagaaccttcggtttggagtacggacggttgggataatttcaagcatagaattaactctgttttggtcgtagatatgccggaggtacaggtaggagaaaagccagagccttattttttttggttgcctatcgcaaacgtactttcttatcgcacatgtgggaaagactgtaatttgatggcttatatcatgtacgtaacggacgaaaataagatcgtctatattgatgaagaacgttatgtaagatttgataaaacgagggaaaacgacttgattttagaggttgacaatatgcacgatttgggctattgtccggctcgtttcttttggtctgactctatatcactgagtgagccggatattaaaataagcccgataacgagcgaactcgactctttcgactggtatctttattattccactgcaaagaagcatttagatttatacgcgtcttatccgatttattccggttacgaacgtgattgtcactatgagtcacacgatggcaaagaacggtgtgatgatggttttttaaagaacgaaaaaaacgagtggataacaggtgcggacggaaaaccgatggcgtgcccgatttgctcaagcaagcggttgaggggcgcaggctcttatgttgagatacccatcccggacgaaatgcacaacgtccccgacttgaaaaacccgatcactatgctatcggctgataccggatcactcgaatataacgtaaacgaggaaaagaggctgagagaggaacttgtaagatcgataacgggtggagaaggggaattaaataggtctgaggctattaacgaaaagcaagttaaagcaggctttgagtccttgactactaaactaaacagaatcaaacgaggcttcgaggaagcgcaaacattcgtagactctactatctgtttactccgttatggtgatagctttgtttcttgcaagattaactacgggactgagttctatatctatacaccggaagagctttcagagcgttataagataatgaaggagaccggagcgtccgaagcggagcttgacgccttaaggcaacaaataatcgaaacggagtaccggaacgatcctacgcagatgcaaaggttattaatccttaacgagatagagccttattcacacttaacgagggaagaagcggtaaatctgtataaagaaaacgttataagtgaggaagatttgcgagttaaattaaaccttcctacatttgtgcgtagatttgaaagagagaacatgaatatcattgagttcggttctgcacttgactataaaaagaaaattgaaataattattaacactttaaaaaagtacgcaaatggtttacagaactgatcagttagatcaactgaatgaaagtaattacgtttgcccgcaggatgaagttaaattgtatcacgttattcaagaagtgaaagaatttaatccgaaaacagggcaaagaatcagcgtcccggtgttgcaaaaatacaagcgaaagacttttgaacttgatattttgccgagactgccaagattgggttatacattgagagttgttttcgacccggttaaatatgaatctacaatttcggaggcaagacgagcagcagaactggcagcgagagccgaggcaaaaatgaaggcagacgaagaactgagagagcaaattagacgtgaagaagctgcaaaacttcgtgcggagttgaagaaacaaaaagagaaaggagaaaagtaatgttaacagtagatttgcttagacagaataaagcgttatcggagctatcggatgaagttcttaacgctatttcagaactttcaaaaaacgatgaagcgcagacggttgcggcaaaggtcagagaaaccgaaaacagtattgcgactcaaatgaaagaggcttttggcattgaaggtgtaaccgatcttgatttgaaaaccgcaattgagtttggaaaaacaaagatttctaaatcggatacatcggcttttgaaaagcagattaacgatctgaaagaagagctaaaagcggagagagccaaaaagggaggcgaacgggatacagataaaatcaatcagcttacagccgaactaaacgacaccaagcaaaaatttgctgagttgaacaaccaactttcagagaaagaaaaggagtttaacggtaagttgaacgattacaagatcacttcttacatttcaagcgctatgcaggggatgaagtttaagaaagatatttcagatccagttctaaacgttgtgaagcagcaggcggttaacttgcttaaaactcaattctcacccactttgcaaggtgacgaaggttctgaaagtcttattttcatgaaagacggtgttccttacaacaaccctgcaaacagtctgaaaccgtttaccgcatcagaacttctgtctcaacagtttgagcagttcggtgtacttgataaaggtagacaggctggcggtgcgggaagttccggaggcggacagggtaacggtagcttgcttgatttaagcggttgcaaaaccaaagtagaggcaaacaaggttgcgcaggagtatttagctaagaaaggttatacaagcgagtcagaagagtatcaaacggagcttgataaaatttgggttgaaaacaagatcgcagatttgccaacagaataactaaagagggggttaaacccctcacaatataaactttaaaacaatagatttatgtcgttaattgctacaagaacacaggagtttagattaaagaaccctaacattgacaaaaatatggctcgcatgaccgaatggggtgcgtatgacttctttttgtctcaaacaaatgcgatggactcaatgctttccgatgaaactaagcgtagagcgttcgcctcaatgggaagcgatattaagattcccgtaattgattacgataaaagcgtaactgtgtcaaacgcccgcacatgcgttattgcagatgcggaaaacacctcacgtctgatcggtgtaacttggaaaacctacgcttttggttttaccatgactccgaacatgtattcaaacaacgaaatcgattaccaacaggactggaacagaaagctacaaaagcacatccgtaagttcatggataccgttgataaggacgctattgcggctttggaggcaaacaaaacacaggtattcggaaacttgctgtattacacaaaaacgggtaacgatgtgcaagtgaaattcactcagcgcaacgacattctcagcgacttgcacccgatgttccgtgcaaacgactattccggtcaacttcatatcattggcgacactggcgtagactcaatgttgcgtaaactggaacagcacggtttgtacaatgacgttaacaaacagttggagtatgcaaacaaagtgttccatttcaccaacaacatgactttagagccggataacttcgctcagatgtatgctgttgagtcgggtaacgttgggttgttgacccgtgtagaccgtgcagcctacaacaacactaagtcgggcacgcatgaatttggaaaggttgttcttccttatttcggtaaagaggttggaacacactactacgaagaagtgggagatcagtcagctatcgcaggcgcagcaacagccgatatgacttgtgacgttaaacatttctacggtttctcagtagatattgctttcgtagtggcttttaactccgatcctgcaacaatcgccaacccaattatgaaggtcgaagtaaacaaagaaaattcgcagtttggcggtactccggtatttatcaccaatgcagatcagatcggtggaggttctccggctggcgaattatcggttaaccttgctaaaatcggaggtagtccggttgctgaatctgctttgaaagtagatttagataaagtcaaaggtgcagcggtttcggctactggtggcgtagttgatgttaaagtcaattcgcaggctgcaaatctgaatgttgaggtgaagaactcaacagattcacccgttaacacaaaggaagttccgggagcgtaacgagaaagtaaactaagtattaacaaagggagggggacaaaatcccttccctttttttatttataaccatgtacagattaaaggatatacaaaaagaacttgctacgctcgtaggatggcggcagtcgtacgatagagacgctaagatagacgaaagtttaacggtgtccgatagtggtgttatgtttcaagacgttcacccacttgtgacgctaagaaacattgaatctattatgccacttgattactatttacgttatccggagtatcgggataccgacacttataagacgggtgacaaggtagtttacggcaaggacgtgttaacgcttcgtccggacgtatgggaggcaataacagagaatgttggtgtagagccttccgatggtgataactggaaacggtacaacccactaagcgattatttgcgtgaattgaacgaaagagcgatcaccaataccgttactcgcttcattaatgaaaagttgattgcaggggaaacaaagacgcttttagagcgtacaaacttcttcgatggttcggggaagataaataacgagattgaccctaccgatagtattgtaggatatgaaatattgccagtccgttctatgggggtaacaaccaagatcgagaagataggtttgcagtttaacaagccgggaaaggtaaaactttaccttatgcacacctcacaggtagacccgattaagacgttcgatttgaattatactaaaaatggttcttatcaatggtttgatgtcggtaacgatgtgttactcccttatatgtctgaggaaacttcacccggtggcttgtggtacttgtgttacgatcaaaaagaattgccgttggggatgtatgctataaacgtctctaaggacttttcacgtgacccgtgcggtacttgtaatatcggaagcgtgcaggcgtggagagagctaacaaagtatatcagagtgtcaccgtatagagttgactctacgcagtcggaggatggcgtaaagatgtggaatatagaaatgaacatgtatacgtctgcaatatgctacggtttgaacgttcaattatcggtaggatgtgatataactgactttatcattcagtctaagtatgccttcacgcatgccgtttctctgcaaatggcttcttatgtgctgcgggagcttgcattaaatccgaacgttaggcaaaatgccaatcaattgaatatcgaccgtgaaacgctattgtacgaagttgacggaaactcacagggacgtgcgcagggtatcggatacgaactaaagaaggcttttgaggctctttctattgatacaaaagggatggatagaatatgcctttcttgccggaacaacgggataagatttaaagcaacatgataagcggactaatagataagtttaagaaggtaggtgaggaactcgacaccggagagatagcaaaaaagattgtgcgtgacaatgataacatacttatcgacatgaacgcacaagatcagctatacgccaagggcgttaaccgtttgggtgttcgtatagacgaataccaaccctaccgacccttaactatacaagtcaaaatagaaaagaggcaaccgtataaccgggtgacgcttaaagacacaggggagttttacgactctttttatgttgagacggcagaagatcggttttacataaaagcctcagatgaaaaaactaattggcttatcaaaaaatacggtgctgaaattttcgggttaacaagtgattcacttgctgagtttattaacgattatgtgaaggacgaagcatataacagagtaaaggagatattaaatgaacgatagggctataattagaccaaatgcgacacttttcgataaaacgatagccgatatacaggtaagcctaacaaaatcgcttaaatggcttaatttcgctttcgggaacgtggttaaattggtagagagaaacgagaggggaaaatttgttaccccatcagtgtattttaagggaaatgattatttgcgcttagagcctgacgataagcggggtaacgtttgctttttctacatgcacgactcacaagattatgaagggggagactctttgtctggctttggcgatctgagggggacggttagcattatcttttggttcgatacccgtaaaataccgggggcagaatattacaacgtggagtttgtaaagtccgaaatactgagagccttaacgcacgaactttatctgccatccggcgatatacaggtgaggaagatatttcatgacgccaataatgtatacaaggagttttctatccaaaagacggataatcaatactacgtttatccctatgcgtgtttgcggtttgagtgcgatattcattgcgaagaagggtgttattaaagggggagtttcccccttttgtgttaaatacatgttaaaacttaaagttttgcttgcaatattaaatagactccttatatttgcaatgtcaaacaacgaaagtagtaacaattaaaaatcaagattatgacagcaattgaagttaaagaagtattagcgaacaacagacagttagttattagctatttcaatgaaaatgttaagcaagatagtttctataatcttggttggtttatgactcgtattttgcaagaatctgaaatcgtttggagaagaagaaaaaacgtatcagaaaaagaaatcatgtctgttatcgcaaagatcatgaaacaatatccccaaatagcaaagaactacgttagcaacattgaaaaggctgttaactatttcggagaggataaagtaaaacaaatgatgaatatcaagtaacatacaaaaagccatgaaagtagataagtatctaaagagccacaaggcaaacgaattttacgtgaaaaagtgtagaggttattatctcgttatggatagctatgataaaagtttggcgtctatggaggttacagaagaagaagctaataaggtagccgaacagcttaataacattcgcaacgaaagattaaacctaacagttaaataatagttaaatattaaatttaaacttgcaatattaaatcttatccttatatttgcagtgtcaaaaggaaacaaattactaacaattaaaaaataaaaattatggcagcaagtttatttattcagagaacaattgaaaaattcgtaatgattgaatttgttaagggaaacatggataccaaagaacaggttgataaaatgatagaggttatccaaagaaaattaaacttttcgcatgaagaagcatgcgactttataagaaaagcaattggaataaacgaataactttaattattaacaggtgggggtaacaccccacataaaaaaacaaaaatatgactacttacatttataaaggacaaaagataagccactccaaaatattatccctattgcgtagtgcaggcatttacggaggaaacaagctatcatattatgaagttttggttaaagctgccgaaaacggcaacgaaagagccacttatattttgagagatttaaaggtgatataataaccgtgggaaaccacacaaattttaaaggtatgtttaacaaagaaagaattgagaatttagaaaagagagttaaggaactggaaagaaaagaaggtatttcaaaactttcggaagatgtatctaaaaggttgatacatgctttaattgactctaacaacgaagtagtaaaaagagtttttgatgatgtgtgtgtccgttcggtatatggtatatctgtgtttcagccacctaaagccggagacattcacgcacgtaatgagttgtttagtggagaggtgttcaaacaagcggcgtttaacggtatacccgattatttaaagaaggtagaagaggataagacagaagcacctaccattgcaagcgtgttggaaaaagccaaaaagaatacgatggcgatacaggagcttttaaaacgaaccggatgttcgaacgttaacgaagtgataagcaagtttgaacttggagtttcttacgaaaagatgtacaatgaacaaatatcaaaaagaaaagagttgttagcagcaacagggtgtgcaaactttagcgacttgaaaaataagttcttatgtaaggatataaagagggatgaactaatagcggaaataaacgagcttgcacgacaaagagaacttttaaatcgtgatttaagaaatcaaatagctaaactttctgataaaaaccaatctttgatgcaaagtgaaaaaagccttatttgcaaacttgcagaaaagaacgaggaattaaagagggtggaggaaatttcagaaggtaggtatgaagaaaatgcctggcttcgtggcgaaatgaaaaagacagaacgagcagtagagcgtctaaagaaggaaaacaaacagcttaaatcggttaatgcggaaagggtaacaagtacgttagatgccatttgccgggaagcggatgcggtagtaggatattcagaattgaaaaagagatgtgaatatttggagagagataaaaaaacattgcttgcttctgtgaaagagttacaaaaacaagtgtttgatatttcgagagataaaaaatacttggagatggcaaacgcttcactcctgcaagaaactcgtaaaattagagagtctttgaatgaaagaatcaagaagctaagacagagacttaaaaaatcatctatccgttacagagacttaaaagaaatcatttcgcacaacggtttgaaatcagtataacaataacagccgggataatatcccggcacaatattaaaagatatgttgaaggtagatataagaaataggatattaaaaccaaaggttggtgagattataaccgtaataggagatatatataccacggtagcggaaactataccggacgaaaacgggagatgtgagtcatgtgctttcaatgatgaaacaaaggtaggaggcgattgtggtgattttgtgagttgctctaaacttaaccgtaaagataatgttatgtttaaactaatagaaagaaagagaactaaggaggttgaaaatgaatagaatatctttgtctgatagagatagattcgtaccgaaagagggtgaagtgttttttgcggaagttccgggaaaaggaatagaccggaaggtagaggcggtattattgaaagacaatagcggttgcaagaattgcgtattttttcaaggcgaattaaaagacttgtgtatgcaaataaactcatttcatcaaaatcatttcgaacgtatggattgttgtgtagcgaataaatgcgtagcttatttcagagatacagaacaagacgatgtatattataaaccattagcggaggtatcagaatgaagcaaaagaaagtgagagattttgaggtgttcaaagtagtacacccgatcacagcaaacgaagtaacaattcaagcaataccacgggatacaatttcatgcaacggatgtgccttccgaaagggagatttagaaagcatgtgtaaagcatatctgtgctttagtgaaagaacattagattgtttagtgttcaaggtagtaaaggagaaattcaaacgaaatgttacagagttttaaaagttaaagtattaatttaaatgtgttgacttatgaacaaaaaagccattgacagcctcttagaggcaaaaagacagattgacgatataattactcaacttatcagagaaaacgaagcagagggcaaaggaattatgtctgcgttgagtaaatgcgaatatatgctattaggttcttgcattatcttcccgcagcaatttatagactggtgtttatcccgtggctttttgtctaccgccactgaggaaaattctctttttggaggttgttatacgattcaaagatatgtgtcaagaacagaccttatccgggtgaatggaagcgatataacctttcatccttcattgttgtacgtatacttacacagaggtgaaaatgaataatacaaaggactgggaggaataccgcaaagatgtgagttactccgaaaagttggacgaatatatcgccaatatgatagactacgatgaaagggagaaactaaagatacttttagatatttgtgagaaaagcaaggataacagctacgatctgcctatcgactgcaaaatataccttcctttaggctctttgttgtttgaacatgaaatgtttgattttataacgtgggctgataagatgggatatattcggtgtgaagaagacaagattatcattatttcgtctatgattaaaaggcggcttatcgttggatcgcttaaagttatgccggagattgtggaagcgttcgttttatacagaaagcatgtaggttaggagtcttttagactccttttcttatttataaacatttcgtttttatccaccttcggagagttcgagactaacgttttaataatcaatatctttgcaaaattgctttttattcatacttttgtacaaactaatatttgaattatggagatttataattattttctttcttgcgtgctacttgtttcgtttgtagcggcattttgtgttaactttgcccgaaagacgggtgtaattgaacggatgtcagtgttcggtgacgcttggttatctaaggtgttccggtggtatggcgatagatcacttattaacgagctaaccaattgcaatttctgcctatcgttttgggcatgtgttatttgttcggtgattgtgtcggtcggaacgctaaacccggtgttcatacttacacccatctttgcaacacctatttgtagaattttaatttaatgattatggagattagaaattatgtatcggttataccacctttcgagatcgtgaaggcggttaagtttaacggtgatgttcacgaattagcgcagctattgccaagttttgaactactttccgcaatggatggtgtgatgatggcacgaataaatgataacgctttccgtgtgtttgataacgactatatcgttcttggcgaaaatgttacttactcagtcgatgaagaaacgtttgccatattatacgagcaggcagataaggaggtgacgaatgaacacgattaaggtaggtaatcacacggtaacggtatacgaaggcattgacgaaatgcctatcgtccgttatcagaagtttaaccgtcttatgctgattgagtcgggagtcggaagtactatcgaggaactcgacacgcatttgcaacgtgcaataatctattgcaggactcagccggaacatacgtataacgagctaatgaatctaaggcagtgtttcaacatggcagcgaatggcgtacatcccggaatgatggcttttgccgccttcgttaagtcggtcgatggcgtggaatatccggttaacgcatccgattccgatctaaaggcgatatttgacagcctcagcgatgcaactattaacgaactttctgatccgtttcagaaggtcaaaaaaaaaatagaggcggaagtatcggtatacttcccacggatggcggacgatcctctgattaaggagtattacgatattaaattgtcgctgataaaggcgaagttagataaacttgtgaacaacgtagataacagtgaggcggtgaaggaaatagaggataagttactaaccttcttcccgcctcgaatattctacgggactgattcggtcgaaataaaaacggataaggagttccaagaaatgtgcttggttatcacgcagaatatgcacataaatgcacgtgaaatgtcggtgtctgagttttacaccgctttcgaaatgattaagagacaggcaaaaaggagtaagaacaaataaatttaaatcaa

(19) United States
(12) Patent Application Publication
(10) Pub. No.: US 2026/0273002 A1
(43) Pub. Date: Sep. 17, 2026

(54) BACTERIOPHAGES AS A TOOL TO MANIPULATE GUT COMMENSAL IMMUNE-MODULATION ACTIVITY

(71) Applicant: TECHNION RESEARCH & DEVELOPMENT FOUNDATION LIMITED, Haifa (IL)

(72) Inventors: Naama GEVA-ZATORSKY, Haifa (IL); Tal GEFEN, Haifa (IL); Dana KARITI, Haifa (IL); Nadav BEN-ASSA, Haifa (IL); Robert ABBOUD, Haifa (IL); Shaqed CARASSO, Haifa (IL); Rawan ZAATRY, Haifa (IL)

(21) Appl. No.: 19/167,103

(22) PCT Filed: Mar. 22, 2024
(86) PCT No.: PCT/IL2024/050300

§ 371 Date: Sep. 19, 2025

Related U.S. Application Data
(60) Provisional application No. 63/491,919, filed on Mar. 23, 2023.

(51) Int. Cl. A61K 35/76; A61P 31/04; A61P 37/04; C12N 7/00

(52) U.S. Cl. CPC A61K 35/76; A61P 31/04; A61P 37/04; C12N 7/00; C12N 2795/10321; C12N 2795/10333

(57) ABSTRACT

The present disclosure relates to the microbiome field. More specifically, the present disclosure relates to bacteriophages as a tool to manipulate gut immune-modulatory activity and uses thereof for therapy. More specifically, the present disclosure provides methods and tools for modulating an immune response in a subject in need thereof, by applying bacteriophage that modulates the phenotype of at least one bacteria in a subject.

### TECHNOLOGICAL FIELD

The present disclosure relates to the microbiome field. More specifically, the present disclosure relates to bacteriophages as a tool to manipulate gut immune-modulatory activity and uses thereof for therapy.

### BACKGROUND ART

References considered to be relevant as background to the presently disclosed subject matter are listed below:
1. Jiang X, Brantley Hall A, Arthur T D, et al. Invertible promoters mediate bacterial phase variation, antibiotic resistance, and host adaptation in the gut. Science (1979). 2019; 363 (6423): 181-187. doi: 10.1126/science.aau5238
2. Ikeda J S, Schmitt C K, Darnell S C, et al. Flagellar Phase Variation of Salmonella enterica Serovar Typhimurium Contributes to Virulence in the Murine Typhoid Infection Model but Does Not Influence Salmonella-Induced Enteropathogenesis. Infect Immun. 2001; 69 (5): 3021. doi: 10.1128/IAI.69.5.3021-3030.2001
3. Mazmanian S K, Round J L, Kasper D L. A microbial symbiosis factor prevents intestinal inflammatory disease. Nature. 2008; 453 (7195): 620-625. doi: 10.1038/nature07008
4. Surana N K, Kasper D L. The yin yang of bacterial polysaccharides: lessons learned from B. fragilis PSA. Immunol Rev. 2012; 245 (1): 13-26. doi: 10.1111/j.1600-065X.2011.01075.x
5. Dasgupta S, Erturk-Hasdemir D, Ochoa-Reparaz J, Reinecker H C, Kasper D L. Plasmacytoid dendritic cells mediate anti-inflammatory responses to a gut commensal molecule via both innate and adaptive mechanisms. Cell Host Microbe. 2014; 15 (4): 413-423. doi: 10.1016/J.CHOM.2014.03.006
6. Round J L, Mazmanian S K. Inducible Foxp3+ regulatory T-cell development by a commensal bacterium of the intestinal microbiota. Proc Natl Acad Sci USA. 2010; 107 (27): 12204-12209. doi: 10.1073/PNAS.0909122107

7. Chang Y C, Ching Y H, Chiu C C, et al. TLR2 and interleukin-10 are involved in Bacteroides fragilis-mediated prevention of DSS-induced colitis in gnotobiotic mice. PLOS One. 2017; 12 (7). doi: 10.1371/JOURNAL.PONE.0180025
8. Nadav Ben-Assa, Michael J. Coyne, et al. Analysis of a phase-variable restriction modification system of the human gut symbiont Bacteroides fragilis. Nucleic Acids Research. 2020 1-14.
9. Shkoporov A N, Khokhlova E V., Stephens N, et al. Long-term persistence of crAss-like phage crAss001 is associated with phase variation in Bacteroides intestinalis. BMC Biol. 2021; 19 (1). 787 doi: 10.1186/S12915-021-01084-3.
10. Mazmanian S K, Round J L, Kasper D L. A microbial symbiosis factor prevents intestinal inflammatory disease. Nature. 2008; 453 (7195): 620-625. doi: 10.1038/nature07008.
11. Surana N K, Kasper D L. The yin yang of bacterial polysaccharides: lessons learned from B. fragilis PSA. Immunol Rev. 2012; 245 (1): 13-26. doi: 10.1111/j.1600-065X.2011.01075.x
12. Ben-Assa N, Coyne M J, Fomenkov A, et al. Analysis of a phase-variable restriction modification system of the human gut symbiont Bacteroides fragilis. Nucleic Acids Res. 2020; 48 (19): 11040-11053. doi: 10.1093/nar/gkaa824.

Acknowledgement of the above references herein is not to be inferred as meaning that these are in any way relevant to the patentability of the presently disclosed subject matter.

### BACKGROUND

Phase variation is the process by which bacteria undergo frequent and reversible genomic alterations in specific loci of their genomes [Moxon R, et al. Annu Rev Genet. 2006; 40:307-333]. These genomic phase-variable alterations can be manifested by genomic sequences flanked by inverted repeats which induce either ‘ON′V′OFF’ switches of gene expression [Krinos C M, et al. Nature. 2001; 414 (6863): 555-558] or alterations in the transcribed sequence and thus the expressed protein [van der Woude M W, et al., Clin Microbiol Rev. 2004; 17 (3): 581]. Analysis of bacterial phase variations can provide new insights into bacterial adaptation to its local environment [1]. Phase variation often influences the production of extracellular surface components presented on the bacterial outersurface and hence exposed to the host. Therefore, such components can confer different bacterial functional phenotypes affecting the host, including immune evasion [2] immunomodulation [3] and more. The prevalence of inversion mediated phase-variable regions is high in host associated bacteria [1], mostly in bacteria from the Bacteroidota phylum-a prevalent phylum in the human gut.

Bacteroides fragilis, a common resident of the gut, can modulate its surface by expressing different polysaccharides (PS, denoted PSA-PSH). Seven out of eight distinct capsular polysaccharides of B. fragilis are regulated by phase variation in an ‘ON’/′OFF′ manner [Krinos C M, et al. Nature. 2001; 414 (6863): 555-558]. Studies have shown that the B. fragilis polysaccharide A (PSA) can modulate the host immune system [3,4], for example, by inducing regulatory T cells (Tregs) and secretion of the interleukin (IL)-10 anti-inflammatory cytokine. Moreover, PSA was shown to confer protection against experimental colitis [3, 5-7] and thus is regarded as an anti-inflammatory polysaccharide.

Inflammatory bowel diseases (IBD), ulcerative colitis (UC) and Crohn's disease (CD), are multifactorial diseases, characterized by a compromised mucosal barrier, inappropriate immune activation and mislocalization of the gut microbiota [Ramos G P, et al. Mayo Clin Proc. 2019; 94 (1): 155-165; Guan Q. J Immunol Res. 2019; Corridoni D, et al. Immunol Lett. 2014; 161 (2): 231-235; Ha C W Y, et al. Cell. 2020; 183 (3): 666-683.e17]. The cause of IBD is still unclear, but genetic, immunological, and environmental factors contribute to the risk of the disease. Since the gut microbiota is considered a major environmental factor, which resides at the site of inflammation, IBD has emerged as one of the most studied diseases linked to it. The inclusion of a mechanistic understanding of bacterial functions and potential functional alterations is necessary to elucidate the role of the gut microbiota in IBD.

A different type of phase-variable system of Bacteroides fragilis is a Type I restriction modification system (R-M). Reversible DNA inversions within this R-M locus leads to the generation of eight specificity proteins with distinct recognition sites [8]. By creating mutants, each able to produce only one specificity protein from this region, the R-M recognition sites of four of these S-proteins were identified [8]. Transcriptome analysis revealed that the locked specificity mutants, whether grown in vitro or isolated from the mammalian gut, have distinct transcriptional profiles, likely creating different phenotypes [8]. Analysis of bacterial phase variations can provide mechanistic insights on microbiota-host interactions, with potential clinical implications.

### General Description

The inventors identified multiple phase variable genomic regions correlated with disease states including the anti-inflammatory polysaccharide-A of B. fragilis, which is turned ‘OFF’ under inflammation. The inventors show here that filtered fecal extracts of IBD patients can alter the phase-variable state of the PSA promoter of B. fragilis, and that not only bacteriophages are correlated with its ‘OFF’ state (analyzed from patients' databases) but also a specific lytic bacteriophage of B. fragilis can trigger the PSA switch and consequently the host colonic Tregs levels decline. This elucidates the bacteriophages as a causal link that consequently alters the host immune system state. These findings shed light on the dynamic interplay between gut inflammation and bacterial phase variation, with potential implications for both diagnosis and treatment of IBD.

A first aspect of the present disclosure relates to a method for modulating an immune response in a subject or host in need thereof, and/or the immunological state of the subject. The method comprising the step of administrating to the subject at least one bacteriophage, or a cocktail of two or more bacteriophages, that modulates, or is associated with modulation of, the phenotype of at least one bacterium in the subject. In some embodiments, the bacteriophage used in the disclosed method may be characterized by at least one of the following features. In some embodiments (i), the bacteriophage modulates, or is associated with modulation of, phase variation of at least one locus (e.g., gene locus) of the at least one bacterium in the host. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject. A further aspect of the present disclosure relates to a method for treating, preventing, inhibiting, reducing, eliminating, protecting or delaying the onset of at least one immune-related disorder in a subject The method comprising the steps of administering to the subject a therapeutically effective amount of at least one bacteriophage or any or any vehicle, matrix, nano- or micro-particle comprising the same, and/or any composition thereof. In some embodiments, the bacteriophage used in the disclosed method may be characterized by at least one of the following features. In some embodiments (i), the bacteriophage modulates, or is associated with modulation of, phase variation of at least one locus (e.g., gene locus) of at least one bacteria in the subject. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject.

A further aspect of the present disclosure relates to a therapeutically effective amount of at least one bacteriophage or any or any vehicle, matrix, nano- or micro-particle comprising the same, and/or any composition thereof, for use in a method for treating, preventing, inhibiting, reducing, eliminating, protecting or delaying the onset of at least one immune-related disorder in a subject. In some embodiments, the bacteriophage used herein may be characterized by at least one of the following features. In some embodiments (i), the bacteriophage modulates, or is associated with modulation of, phase variation of at least one locus (e.g., gene locus) of at least one bacteria in the subject. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject.

A further aspect of the present disclosure relates to an engineered bacteriophage comprising at least one exogeneous recognition sequence of at least one bacterial restriction modification system (R-M) of at least one bacteria. The at least one recognition sequence is specifically recognized by at least one component of the restriction modification system of at least one bacterium displaying a desired phenotype. More specifically, the recognition sequence in the bacteriophage comprises a 5′ recognition site and a 3′ recognition site separated by 3 to 10 unspecified intervening nucleotides.

A further aspect of the preset disclosure relates to a composition at least one engineered bacteriophage or any cocktail or mixture of the bacteriophages or any vehicle, matrix, nano- or micro-particle thereof. The engineered bacteriophage comprising at least one exogeneous recognition sequence of at least one bacterial restriction modification system (R-M), wherein said at least one recognition sequence is specifically recognized by at least one component of said restriction modification system of at least one bacterium displaying a desired phenotype. The recognition sequence comprises a 5′ recognition site and a 3′ recognition site separated by 3 to 10 unspecified intervening nucleotides. In some embodiments, the composition further comprises at least one of pharmaceutically acceptable carrier/s, diluent/s, excipient/s and additive/s.

A further aspect of the present disclosure relates to a method for modulating the phenotype of a bacterial population. The method comprising the step of contacting the bacterial population with an effective amount of at least one engineered bacteriophage comprising at least one exogeneous recognition sequence of at least one bacterial restriction modification system (R-M). It should be noted that the at least one recognition sequence is specifically recognized by at least one component of the restriction modification system of at least one bacterium displaying a desired phenotype. The recognition sequence comprises a 5′ recognition site and a 3′ recognition site separated by 3 to 10 unspecified intervening nucleotides, thereby enriching bacteria displaying the desired phenotype in the bacterial population.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1I. Evidence of viral association to B. fragilis Polysaccharide A's promoter genomic orientation

FIG. 1A. Experimental design of culturing B. fragilis in patient's fecal filtrates.

FIG. 1B. Ratio of the ‘ON’ orientation of the PSA promoter of B. fragilis, measured by qPCR, after ex-vivo exposure to fecal filtrates of CD patients before and after treatment with anti-TNF (either Infliximab (HR) or Humira (HuR)). Data represent the median (line in box), IQR (box), and minimum/maximum (whiskers). (One-sided Wilcoxon rank sum test, *p<0.05). Dots represent individual experiments; lines connect experiments from the same patient; Shapes are determined by the patients' treatments, circle: HR, triangle: HuR.

FIG. 1C. Calprotectin levels (μg/g) measured in patients' feces. Data represent the median (line in box), IQR (box), and minimum/maximum (whiskers). (One-sided Wilcoxon rank sum test, *p<0.05). Dots represent samples; lines connect samples from the same patient; Shapes are determined by the patients' treatments, circle: HR, triangle: HuR.

FIG. 1D. Volcano plot of differential viral taxonomic units' abundance, (from the IBDMDB cohort, count table from [Nishiyama H, et al. Microorganisms. 8 (11): 1-15. 2020]), between samples with low ‘ON’ orientation of the PSA promoter (<40%) and high ‘ON’ orientation (>60%), in the IBDMDB cohort count table. Differentially abundant viral taxonomic units were detected by the DeSeq2 algorithm (Wald test, p<0.01). Empty dots indicate differentially abundant bacteria that were determined by adjusted P value <0.01 and log 2 fold change >−1.5 and <−1.5, respectively.

FIG. 1E. Phage to host ratios of viral OTUs detected in FIGS. 1D and 1G.

FIG. 1F. Phage to host ratios of viral OTUs detected in FIG. 1D against samples' levels of PSA's promoter ‘ON’ orientation analyzed from the IBDMDB cohort, based on the count table from [Nishiyama H, et al. Microorganisms. 8 (11): 1-15. 2020].

FIG. 1G. Volcano plot of differential viral taxonomic units' abundance, (from the IBDMDB cohort, count table from [Nishiyama H, et al. Microorganisms. 8 (11): 1-15. 2020], between samples with low ‘ON’ orientation of the CPS3 promoter (<40%) and high ‘ON’ orientation (>60%), in the IBDMDB cohort count table. Differentially abundant viral taxonomic units were detected by the DeSeq2 algorithm (Wald test, p<0.01). Empty dots indicate differentially abundant bacteria that were determined by adjusted P value <0.01 and log 2 fold change >−1.5 and <−1.5, respectively.

FIG. 1H. Phage to host ratios of viral OTUs 0.791, 0820 and 1202 detected in FIG. 1D against samples' levels of PSA's promoter ‘ON’ orientation analyzed from the IBDMDB cohort, based on the count table from [Nishiyama H, et al. Microorganisms. 8 (11): 1-15. 2020].

FIG. 1I Phage to host ratios of viral OTUs 0165, 1130 and 1490 detected in FIG. 1G against samples' levels of CPS3's promoter ‘ON’ orientation analyzed from the IBDMDB cohort, based on the count table from [Nishiyama H, et al. Microorganisms. 8 (11): 1-15. 2020].

FIG. 2A-2E. Characterization of Bacteriophage Barc2635

FIG. 2A. A representative image of plaques from a plaque assay of BARC2635 on B. fragilis NCTC 9343 on BPRM Agar plates.

FIG. 2B. A representative transmission electron microscopy photo of Bacteriophage Barc2635.

FIG. 2C. The genome structure, GC content and putative annotations of BARC2635. CDS: coding sequence.

FIG. 2D. Adsorption assay of BARC2635 bacteriophage.

FIG. 2E. One-step assay of BARC2635 bacteriophage.

FIG. 3A-3G. Phage exposure drives phase variation

FIG. 3A. Phylogenetic tree based on the whole genome of viral OTUs identified as bacteriophages against B. fragilis as well as bacteriophage Barc2635. Multiple Alignment using Fast Fourier Transform (MAFFT) was used to perform multiple sequence alignment. The average-linkage method was used to construct the phylogenetic tree with 1000 bootstrap replicates. Patterns denote association between the viral OTUs abundances in [Nishiyama H, et al. Microorganisms. 8 (11): 1-15. 2020], IBDMDB cohort, with active Crohn's disease (sparsely dotted pattern) or with both active Ulcerative colitis and active Crohn's disease (densely dotted pattern).

FIG. 3B. Experimental design of in vivo experiments.

FIG. 3C. Expression levels of UpaY in ceca of mice monocolonized with B. fragilis vs. mice monocolonized and infected with the Barc2635 bacteriophage. Levels are shown as 2∧(−ΔCT) with RpsL as a reference gene. Each dot represents a mouse. **** P<0.0001, Mann-Whitney test. UpaY is the first immediate gene after the PSA promoter, hence, when the transcript is present—it's indicative of the activity of the PSA promoter i.e. PSA promoter ‘ON’ state leads to transcription of the UpaY transcript.

FIG. 3D. PSA presence on the outersurface of B. fragilis exposed to the Barc2635 bacteriophage, detected by anti-PSA antibodies. Bacteria were analyzed by flow cytometry for the expression of PSA, using Rabbit anti PSA antibodies. Each dot represents a mouse.

FIG. 3E. Percentage of the bacterial population with each of the phase-variable Type-I R-M specificity gene combinations at the expression locus. Each dot represents a mouse. Bacterial Type-I R-M genes amplicons were detected by long reads sequencing (MinIon)

FIG. 3F. CD4+ to CD8+ ratio out of CD45+TCRβ+live cells in colon. Single cells were isolated from colon lamina propria. Immune cells were analyzed by flow cytometry. Each dot represents a mouse.

FIG. 3G. RORγ+helios-percentages out of FOXP3+ cells in colon. Single cells were isolated from colon lamina propria. Immune cells were analyzed by flow cytometry. Each dot represents a mouse. *** P<0.001, **** P<0.0001, one-way analysis of variance (ANOVA).

FIG. 4A-4E. Phage exposure drives phase variation

FIG. 4A. Ratio of B. fragilis PSA promoter reverse orientation in mice fecal samples measured by the PhaseFinder tool, on different days of the experiment.

FIG. 4B. Ratio of B. fragilis PSF promoter reverse orientation in mice fecal samples measured by the PhaseFinder tool, on different days of the experiment.

FIG. 4C. CFUs of B. fragilis in fecal samples of mice monocolonized with B. fragilis (star) or colonized with B. fragilis+phage (circle); PFUs of Barc2635 in fecal samples of mice colonized with B. fragilis+phage (dashed black line only).

FIG. 4D. CFUs of B. fragilis in fecal samples of mice monocolonized with B. fragilis or colonized with B. fragilis+phage (Wilcoxon rank sum test, *p<0.05).

FIG. 4E. PFUs following infection of B. fragilis with or without Barc2635 bacteriophage.

FIG. 5. Flow cytometry gating strategy

Representative flow cytometry plots demonstrating the gating strategy for the staining panel.

FIG. 6. isolated phages from IBD patients drives phase variation in PSA promoter Ratio of B. fragilis PSA's promoter ‘ON’ orientation measured on day 10 by qPCR in fecal samples of gnotobiotic mice monocolonized with B. fragilis and an isolated bacteriophage from IBD patients before receiving anti-TNF treatment. (Mann-Whitney test, *** p<0.001).

FIG. 7A-7B. Phage engineering

FIG. 7A. The Type-I R-M system is to be used for phage engineering.

FIG. 7B. Steps of phage engineering.

### DETAILED DESCRIPTION OF EMBODIMENTS

The gut microbiota has been shown to have a pivotal role in shaping the immune system and host-physiology. Currently, bacterial activity can be modulated by pre-biotic (diet), pro-biotic (live or dead bacteria) and antibiotics. Bacterial manipulations by the known solutions are widely affecting the total bacterial composition and is not limited to the wanted bacteria. Thus, the outcome of such a therapy is hard to predict and may induce various unwanted side effects. Most studies on gut microbiota-host interactions focus on the bacterial component of the gut, neglecting the gut bacteriophages, an extremely specific class of viruses that infect bacteria. Bacteriophages are a crucial component of the gut microbiota that can manipulate the effects of bacteria on the mammalian host. The inventors' results support this hypothesis, showing that bacteria infected with their associated bacteriophages induce a much higher immune response in the mammalian host. Bacterial genomic phase variations are prevalent in host-associated species [1]. This mechanism can allow bacteria to survive in ever-changing environments, such as the human gut. The inventors applied fecal filtrates of inflamed IBD patients, before and after anti-TNF treatment, and demonstrated that these filtrates can modulate the PSA promoter orientation ratios in an ‘ex-vivo’ experiment setup. Filtrates of inflamed patients triggered an OFF-switch, while filtrates of treated patients triggered an ON-switch, in accordance with the patients' stool inflammation levels. Notably, the directionality of the promoter phase-variation aligned in the patients' database analyses, the mice experiments and the ‘ex-vivo’ filtrates experiments.

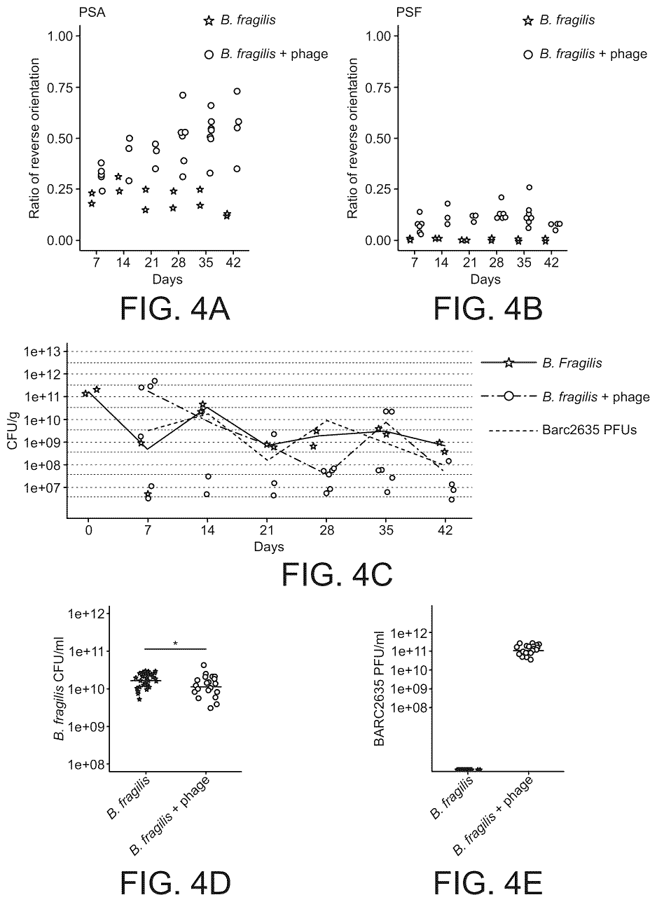

Since the phase variation ratios shifted in both directions in response to stool filtrates, depending on the patients' inflammatory state, the inventors hypothesized that factors in the inflamed gut ecosystem may trigger bacterial phase variation. Stool filtrates can contain various factors such as bacterial and host metabolites, host immune system factors, bacteriophages and more. Recent studies have reported on bacteriophage induction in IBD patients [Qv L, et al. Front Cell Infect Microbiol. 2021; 11; Duerkop B A, et al. Nat Microbiol. 2018; 3 (9): 1023; Norman J M, et al. Cell. 2015; 160 (3): 447-460] and bacteriophages from Caudovirales order were associated with the pathogenesis of IBD [Norman J M, et al. Cell. 2015; 160 (3): 447-460; Zuo T, et al. Gut. 2019; 68 (7): 1169-1179]. Therefore, the inventors sought to correlate between bacteriophage abundances to bacterial phase variations in patients' stools. To this end, the inventors analyzed the viral OTUs in IBDMDB identified in Nishiyama et al. [Microorganisms. 2020; 8 (11): 1-15] in relation to B. fragilis PSA promoter orientation. This analysis revealed specific B. fragilis associated viral OTUs, enriched in samples where the PSA promoter was present in its ‘OFF’ orientation. Moreover, in these samples a higher bacteriophage to host ratios were found. These results suggest that phage expansion in the inflamed gut might induce bacterial phase variation. To examine the possible direct effects of bacteriophages on genomic phase variation in B. fragilis, the inventors co-introduced B. fragilis and its' associated lytic bacteriophage BARC2635 (which was previously isolated from sewage) to gnotobiotic mice. Intriguingly, the presence of BARC2635 triggered phase-variation in the PSA promoter of B. fragilis to its ‘OFF’ orientation. Type I restriction-modification (R-M) systems are systems that potentially can provide protection against bacteriophages, by identifying and restricting foreign DNA. The inventors previously characterized a phase variable Type I R-M system of B. fragilis and demonstrated its phase variation in vivo, in response to the mammalian host. The inventors also showed that phase variation of this system leads to altered transcription of capsular polysaccharides. Since BARC2635 triggered phase variation in PSA, the inventors next asked whether it could also trigger phase variation in this Type I R-M system. It was found that in vivo infection of B. fragilis with BARC2635 skews the bacterial population towards a certain orientation of this Type I R-M system, which up-regulates PSF. PSF was shown to inhibit PSA [Chatzidaki-Livanis, M et al. Proc Natl Acad Sci USA. 2010; 107 (26): 11976-11980], and hence this phase-variation aligns with PSA ‘OFF’ phase-variation, also triggered by BARC2635. Notably, these bacteriophage-induced phase variable alterations in B. fragilis prolonged for over 4 weeks (i.e PSA ‘OFF’, PSF ‘ON’) (FIGS. 4A and 4B, respectively). The anti-inflammatory effects of PSA are mediated by upregulation of Tregs and induction of IL-10 secretion. The inventors therefore examined the colonic Tregs in gnotobiotic mice colonized with B. fragilis and infected with BARC265. Indeed, the Treg cells populations decreased in B. fragilis monocolonized mice infected with BARC2635, suggesting that the PSA ‘OFF’ trigger of the bacteriophage BARC2635 has implications on the host immune system.

The CPS of Bacteroides thetaiotaomicron were previously shown to play a role in the bacterial susceptibility to bacteriophages [Porter N T, et al. Nat Microbiol. 2020; 5 (9): 1170-1181]. Applying the same analysis to B. thetaiotaomicron's CPS3, on one hand the inventors observed bacteriophages that are more abundant in samples with a higher ratio of CPS3 ‘ON’ oriented promoters, however, on the other hand, the bacteriophage to host ratios per sample were too low to conclude that B. thetaiotaomicron bacteriophages could directly trigger the CPS3 phase variation.

As firstly disclosed herein, bacteriophages are an example of an environmental factor that can mediate phase variation. The present disclosure further shows that bacteriophages modulate bacterial functionality. Moreover, bacteriophages were shown to modulate the immune response is subjects suffering from immune-related disorders, thereby supporting a therapeutic application thereof. Taken together, this study sheds light on the potential role of gut bacteria in the development and progression of immune-related disorders, such as IBD. The inventors find dynamic phase variations in multiple genomic regions of Bacteroides species, mostly in outersurface bacterial components and in Type I R-M system, alluding to possible alterations in microbe-host interactions under inflammatory conditions. Indeed, one of these genomic regions, the anti-inflammatory PSA promoter of B. fragilis, was turned ‘OFF’ in inflammation. The inventors find that the PSA ‘OFF’ switch can be mediated by bacteriophages, and consequently the immune system response is altered towards a more inflammatory state.

The inventors' findings provide insights into the dynamic and reversible nature of bacterial phase variation in response to inflammation. The identification of phase variation in multiple species of gut bacteria suggests that this mechanism may play a broad role in the development and progression of IBD. The results of the inventor's study present three major implication avenues: (1) a non-invasive diagnostic measure; (2) potential nodes for future therapeutics; and (3) mechanistic understanding on gut bacterial functional plasticity. Future studies may illuminate the role of phase variation in additional physiological states, and on additional environmental triggers of phase variations, which could drive alterations in bacterial functionalities and microbe-host interactions.

Thus, a first aspect of the present disclosure relates to a method for modulating an immune response in a subject or host in need thereof, and/or the immunological state of the subject. The method comprising the step of administrating to the subject at least one bacteriophage, or a cocktail of two or more bacteriophages, or mixture of the bacteriophages or any vehicle, matrix, nano- or micro-particle thereof or composition comprising the same. In some embodiments, the bacteriophage used in the method is a bacteriophage that modulates, or is associated with modulation of the phenotype of at least one bacteria in the subject. In some embodiments, the bacteriophage used in the disclosed method may be characterized by at least one of: In some embodiments (i), the bacteriophage modulates, or is associated with modulation of, phase variation of at least one locus (e.g., gene locus) of the at least one bacteria in the host. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject.

As mentioned above, the present disclosure relates to a method for modulating an immune response in a subject or host in need thereof, and/or an immunological state of a subject. The term “Immune response” as used herein refers to physiological reactions which occur within the subject or any other host, in the context of inflammation, or immunological reaction. More specifically, the immune response includes the innate and the adaptive immune responses. Components of the innate immune response include immune cells such as neutrophils, macrophages, and monocytes, and soluble factors including cytokines and complement. The adaptive branch include cells such as dendritic cells, T cell, and B cells as well as antibodies (also known as immunoglobulins) which directly interact with antigen. Thus, modulating an immune response of a subject or any other host refers herein to modulating at least one component (e.g. Treg, cytokine, etc) of the immune response of the subject or any other host. Still further, in some embodiments, the present disclosure relates to a method for modulating an immunological state of a subject. “Immunological state”, as used herein, reflects the state of the immune system of a subject. The immune system acts to protect the host from pathogenic agents, biotic and non-biotic stimuli, in the environment (bacteria, viruses, fungi, parasites, toxins, and chemical entities). It serves to distinguish “nonself”′ from “self.” In some embodiments, the immunological state of the subject may reflect a health condition of a subject. A “health condition” of a subject as used herein, refers to the state that subject is in, especially the physical state of the subject, that is not related to any pathology. Parameters such as physical and/or mental functioning, body structure and weight, personal factors (such as age, sex), medical test results, tissue and organ integrity and function, activity and environmental aspects are the composite health or physical state of a subject. This term encompasses any healthy, or non-diseased-homeostatic condition, for example, puberty, pregnancy, menstruation, menopause, aging, obesity, metabolic syndrome and the like. In some other embodiments, the immunological state of the subject may reflect an immune-related disorder as detailed herein after, in connection with other embodiments and/or other aspects of the present disclosure. Still further, the present disclosure provides methods for modulating an immune response. It should be understood that the term “modulating” as used herein encompasses any change or modification to any of the immune response components of a subject or any other host and/or to the immunological state of a subject, and/or the immune system of the subject, or any cellular or non-cellular components thereof. More specifically, components of the immune system as used herein refer to cellular components such as macrophages, neutrophils, dendritic cells, natural killer cells, T cells, B cells, and any regulatory cells, specifically, T regulatory cells, physical barriers such as the skin and mucous membranes, complement proteins, signaling molecules produced by immune cells and other cell types, regulating immune responses by mediating cell-to-cell communication, inflammation, immune cell activation, differentiation, migration, and homeostasis. The term modulation of an immune response or the immune system of the subject may also encompass in some embodiments, changes in the genetic loci encoding molecules such as major histocompatibility complex (MHC) proteins, and the like. In yet some further embodiments, such change may include an increase, enhancement or alternatively, decrease in at least one of the immune response components and/or immunological state in relation to their normal and/or baseline level. More specifically, in some embodiments, “modulation” as used herein may further encompasses changing the nature, the extent, the direction of the immune response in the subject, or changing the amount or function of at least one component (either cellular or non-cellular components) of the immune system of the administered subject. In some embodiments, when referring to the extent or the direction of an immune response, or the level of specific components thereof, modulation as used herein encompasses either reduction or enhancement of the immune response, or components thereof. More specifically, “inhibition”, “moderation”, “reduction” or “attenuation” as referred to herein, relate to the retardation, restraining or reduction of the immune response, or any components of the immune response by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. Therefore, the term inhibit, or decrease refers to an inhibition of about 2, 3, 4, 5, 6, 7, 8, 9, 10, 20, 30, 40, 50, 60, 70, 80, 90, 100, 200, 300, 400, 500, 600, 700, 800, 900, 1000 folds or more, as compared to the level, extent or nature of the immune response prior to the administration of the bacteriophages or without the administration of the bacteriophage in accordance with the present disclosure. Similarly, or alternatively, the term “modulation” encompasses the option that the disclosed bacteriophages increase, augment, boost, expand, enhance, raise, amplify, intensify, strengthen, enlarge, escalate, multiply, the immune response or specific components of the immune system of the subject by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, as compared to the level, extent or nature of the immune response prior to the administration of the bacteriophages, or without the administration of the bacteriophage in accordance with the present disclosure. (It should be appreciated that 10%, 50%, 120%, 500%, etc., as used herein for the increase or alternatively, the decrease, are interchangeable with “fold change” values, i.e., 0.1, 0.5, 1.2, 5, etc., respectively. 10%, 50%, 120%, 500%, etc., are interchangeable with “fold change” values, i.e., 0.1, 0.5, 1.2, 5, etc., respectively. In some embodiments, the method comprising the step of administrating to the subject at least one bacteriophage, or a cocktail of two or more bacteriophages. A “bacteriophage”, also known informally as a phage, is a duplodnaviria virus that infects and replicates within bacteria and archaea. Bacteriophages are composed of proteins that encapsulate a DNA or RNA genome, and may have structures that are either simple or elaborate. Their genomes may encode as few as four genes and as many as hundreds of genes. During infection a phage attaches to a bacterium and inserts its genetic material into the cell. After that a phage usually follows one of two life cycles, lytic (virulent/lytic phages) or lysogenic (temperate phages). It should be understood that the present disclosure encompasses the use of lytic or alternatively lysogenic bacteriophages. As indicated herein, the methods provided by the present disclosure encompass the administration of at least one bacteriophage or any cocktail or mixture thereof. A “cocktail” refers herein to a combination of at least two bacteriophages, 3, 4, 5, 6, 7, 8, 9, 10, 20, 30, 40, 50 or more different or similar bacteriophages. In some embodiments, the bacteriophages may be of different types (e.g. lytic, temperate, etc) and/or different taxonomy (e.g. order, family, species, etc). In other embodiments, the bacteriophages may be of the same type and/or taxonomy.

In some embodiments, the bacteriophage of the present disclosure modulates or is associated with modulation of the phenotype of at least one bacterium in the subject. “Bacteria” (singular bacterium) as used herein are microscopic, single-celled living organisms. Bacteria include Gram positive, Gram negative and Gram variable bacteria and intracellular bacteria. The term “phenotype” as used herein refers to the biochemical and physiological properties of the bacteria, which results from the expression of the bacteria's genetic code (its genotype) and the influence of environmental factors. Both factors may interact, further affecting the phenotype. Still further, in some embodiments, a phenotype of bacteria refers to observable characteristics or traits exhibited by a bacterial strain, which may include but are not limited to morphological features such as size, shape, and color. Growth characteristics such as growth rate, temperature tolerance, and nutrient requirements. Metabolic activities such as the ability to produce specific enzymes or metabolites. Resistance or susceptibility to antimicrobial agents or environmental stresses, as well as pathogenicity or virulence factors, and any other distinctive traits that differentiate the bacterial strain from others. The phenotype may be determined by analyzing the level, activity, stability, and/or post translational modifications of at least one gene and/or gene product of a bacterium. In some embodiments, the indicated phenotype affects the immune response/immunological state of a host/subject. As a non-limiting example, the bacteria phenotype may be determined by the level of at least one of the bacterial capsular polysaccharides (CPS), such as PSA, PSF, CPS3 and more. The bacteriophage used in the disclosed methods modulates or is directly or indirectly associated modulation of the phenotype of at least one bacteria in the subject. The term “modulate” or “associated with modulation” as used herein refers to modulation of the bacteria phenotype either directly or indirectly. It means that the bacteriophage changes the phenotype of at least one bacteria in the subject, e.g., at least one observable, or otherwise comparable parameter associated with the bacteria. In some embodiments, the modulation of the phenotype of the bacteria may be towards a desired phenotype. It means that the phenotype, e.g., expression level, function, activity of the bacteria is changed upon exposure or administration of the bacteriophage to a phenotype associated with at least one beneficial property of the bacteria. In some embodiments, a “desired phenotype” refers to a specific set of observable characteristics or traits that are intentionally directed for a particular purpose. In some embodiments, a phenotype comprises the expression of at least one gene or protein of interest by the bacteria. The change in the phenotype may be reflected by a change in the level of expression, the pattern of expression, post translational modifications, the level and type of activity and the like.

In some embodiments the bacteriophage modulates or is associated with modulation of phase variation of at least one locus (e.g., gene locus) of the at least one bacteria in the subject, or any other host. The term “phase variation” (or antigenic variation) as used herein, refers to a reversible switch between an “all-or-none” (on/off) expressing phase, resulting in variation in the level of expression of one or more proteins between individual cells of a clonal population. Antigenic variation mechanisms generate variations in the sequence of surface proteins resulting in the expression of different forms and structures of the antigenic proteins on the cell surface. Genetic modifications and/or modulation mechanism of phase variation includes for example DNA inversion, DNA recombination, transposition mechanism, slipped strand mispairings (SSM) and phase variation via differential methylation.

DNA inversion is carried out by enzymes that recognize inverted repeat regions and flip the DNA sequence in between them or which reside next to the switch. For example, if a promoter region lies within the sequence flanked by the inverted repeats this leads to shut down of gene expression.

DNA recombination provides a pathway for DNA re-arrangement and subsequent phase variation. Events arising from recombination mechanisms are often due to DNA deletions, and thus tend to be in a one way ON to OFF direction.

Transposition mechanism leads to either insertion or excision of transponsable elements. A transposable element (TE, transposon, or jumping gene) is a nucleic acid sequence in DNA that can change its position (jump) within a genome from one location to another, sometimes creating or reversing mutations and altering the cell's genetic identity and genome size. Slipped strand mispairings (SSM) (also known as replication slippage) occurs during the process of DNA synthesis i.e., DNA replication, repair and recombination. During DNA synthesis template and nascent strand transiently separates from each other and then reanneal. In reannealing step, nascent strand on the template strand can be slipped either in forward direction or in backward direction, resulting in “bulge” formation. The bulge is formed either on the template strand due to forward slippage or in the nascent strand due to backward slippage, leading to either contraction or expansion of the repeat tracts which in turn could affect in many ways transcription or translation of the affected contingency gene depending on the position of the repeat tract. Short sequence repeats (SSRs) are homo- or hetero-nucleotide repeats in DNA that are highly prone to insertion/deletion errors due to Slipped-Strand Mispairings (SSMs) during DNA replication. Phase variation via differential methylation is described as epigenetic event as phenotypes are altered but not genotype, therefore maintaining the integrity of genome (e.g., epigenetic variations). In prokaryotes, DNA methylation occurs mainly at the nucleotide adenine although cytosine methylation can also occur. DNA methylation usually occurs at specific target sites and is carried out either by methyltransferases that are part of dedicated Restriction-Modification (RM) systems or by orphan methyltransferases.

As mentioned above, the bacteriophage modulates or is associated with modulation of phase variation of at least one locus in bacteria. The term “locus” or “phase variation locus” or “phase variation region” refers herein to a part of the DNA that is prone to genetic or epigenetic modifications that result in phase variation. Phase variation regions may comprise various structural properties, for example repeats, inversions, insertions, deletions, amplifications, methylation, etc.

In yet some further additional or alternative embodiments, the bacteriophage comprises at least one recognition sequence for at least one bacterial restriction modification system (R-M). The term “recognition sequence” as used herein refers to a DNA sequence to which a structural motif of a DNA-binding domain (in this case a component of the R-M system) exhibits binding specificity. A given recognition sequence as used herein can occur one or more times on a specific DNA fragment. In some embodiments, the bacteriophage of the present disclosure may comprise 1, 2, 3, 4, 5, 6, 7, 10, 20, 30, 40, 50, 60, 70, 90, 100 or more copies of the same or of different recognition sequences. In some specific embodiments, the recognition sequence comprises a 5′ recognition site and a 3′ recognition site separated by 3 to 10 unspecified intervening nucleotides. Different recognition sequences therefore may refer to the 5′ recognition site, the 3′ recognition site and/or the 3 to 10 unspecified intervening nucleotides.

In some embodiments, the recognition sequence in the bacteriophage is specifically recognized by at least one component of the R-M system of at least one bacterium displaying a desired phenotype and/or a genotype associated with a desired phenotype. A “genotype”, as used herein, refers to the genetic constitution or makeup of the bacteria, which encompasses the specific sequence of nucleotides. The genotype of a bacterium includes all the genes and genetic elements present within its genome, including coding sequences for proteins, regulatory regions, mobile genetic elements, and any other genomic features. Still further, the “restriction modification system (RM system)”, found in bacteria and other prokaryotic organisms, provides a defense against foreign DNA, such as that borne by bacteriophages. Bacteria have restriction enzymes, also called restriction endonucleases, which cleave double stranded DNA at specific points into fragments, which are then degraded further by other endonucleases. This prevents infection by effectively destroying the foreign DNA introduced by an infectious agent (such as a bacteriophage). As the sequences recognized by the restriction enzymes are very short, the bacterium itself will almost certainly contain some within its genome. To prevent destruction of its own DNA by the restriction enzymes, methyl groups are added. These modifications must not interfere with the DNA base-pairing, and therefore, usually only a few specific bases are modified on each strand. Endonucleases cleave internal/non-terminal phosphodiester bonds, upon recognition of specific sequences in DNA which are usually 4-6 base pairs long, and often palindromic. There are four major categories of restriction modification systems: type I, type II, type III and type IV, all with restriction enzyme activity and a methylase activity (except for type IV that has no methylase activity).

Type I systems or Type1RM are the most complex, consisting of three polypeptides: R (restriction enzyme (HsdR)), M (modification: methyltransferase protein (HsdM)), and S (specificity protein (HsdS)). The resulting complex can both cleave and methylate DNA. Both reactions require ATP, and cleavage often occurs a considerable distance from the recognition site. The S subunit determines the specificity of both restriction and methylation. Cleavage occurs at variable distances from the recognition sequence. Type II systems are the simplest and the most prevalent. Instead of working as a complex, the methyltransferase and endonuclease are encoded as two separate proteins and act independently (there is no specificity protein). Both proteins recognize the same recognition site, and therefore compete for activity. The methyltransferase acts as a monomer, methylating the duplex one strand at a time. The endonuclease acts as a homodimer, which facilitates the cleavage of both strands. Cleavage occurs at a defined position close to or within the recognition sequence. Type II systems are typically used in labs for DNA analysis and gene cloning. Type III systems have R (res) and M (mod) proteins that form a complex of modification and cleavage. The M protein, however, can methylate on its own. Methylation also only occurs on one strand of the DNA unlike most other known mechanisms. The heterodimer formed by the R and M proteins competes with itself by modifying and restricting the same reaction. This results in incomplete digestion. Type IV systems are not true RM systems because they only contain a restriction enzyme and not a methylase. Unlike the other types, type IV restriction enzymes recognize and cut only modified DNA. The recognition sequence thus may be recognized by any one of the components of the R-M system (namely, the specificity protein (or the S protein), the methyltransferase (or the M protein), or the endonuclease (or the R protein)). As mentioned above, the recognition sequence in the bacteriophage is specifically recognized by at least one component of the R-M system of at least one bacterium displaying a desired phenotype and/or a genotype associated with a desired phenotype. The term “display a desired phenotype” means that the bacterium which has the R-M system that recognizes the recognition sequence in the bacteriophage, also presents the favorable desired phenotype. For example, if the desired favorable phenotype is high level of PSA, the bacterium which has the R-M system that recognizes the recognition sequence in the bacteriophage also has high expression levels of PSA.

In yet some further alternative or additional embodiments the bacteriophage used in the disclosed methods modulates bacterial functionality in the subject. Bacterial functionality is described herein as the ability of the bacteria to affect the host's physiology. Examples includes the ability to modulate the immune system. The ability to affect gut permeability. The ability to change the functionality of other neighboring bacteria. The ability to cause disease. The ability to influence the efficacy of immunotherapy.

It should be understood that the modulation of the phenotype of at least one bacteria toward a desired phenotype in the subject, as used herein is meant any modulation that occurs in the subject. This term compasses modulation of any bacteria that naturally resides within at least one bacterial population in the subject or any other host, as well as any bacteria that was added to the subject such that the bacteriophage modulates the phenotype of the exogenously added bacteria, in the recipient host.

In some embodiments, the disclosed methods may further comprise administrating the at least one bacteria to the subject. Such additional administration may be applicable where the specific bacteria is not naturally present in the immuno-modulated subject. It should be understood that in some embodiments the administration of the microorganism may be performed prior to administration of the phage. In other embodiments, the administration of the microorganism may be performed together with the administration of phage. Still further, in some other embodiments, the microorganism may be administered following administration of the phage.

As indicated above, in some embodiments, the bacteriophage administered to the subject, modulates the phenotype of at least one bacteria. In some embodiments, the bacteria reside within at least one microbiome community of the subject. “Microbiome community” as used herein is a community of commensal, symbiotic, and pathogenic microorganisms (such as bacteria, archea, fungi, and viruses) that can usually be found living together in a particular environment. Mammalian, for example, human microbiome includes for example gut, skin, genital, and oral microbiome communities.

In some specific embodiments, the bacteria reside within the gut microbiome of the subject. The term “gut microbiota” or “gut microbiome”, or “gut flora”, refers to the microorganisms, including bacteria, archaea, fungi, and viruses that live in the digestive tracts of animals. The gut is the main location of the human microbiome. In humans, the gut microbiota has the largest number and species of bacteria compared to other areas of the body. The gut microbiota has broad impacts, including effects on colonization, resistance to pathogens, maintaining the intestinal epithelium, metabolizing dietary and pharmaceutical compounds, controlling immune function, and even behavior through the gut-brain axis.

Still further, in some embodiments of the disclosed methods, the at least one bacteria affected by the administered bacteriophage comprise at least one bacterium of at least one phylum selected from Bacteroidota, Verrucomicrobiota, proteobacteria, actinobacteria, firmicutes and Tenericutes. The phylum “Bacteroidota” (synonym Bacteroidetes) is composed of three large classes of Gram-negative, nonsporeforming, anaerobic or aerobic, and rod-shaped bacteria that are widely distributed in the environment, including in the guts and on the skin of animals. “Verrucomicrobiota” is a phylum of Gram-negative bacteria that contains only a few described species, which have been isolated from fresh water, marine and soil environments and human feces. This phylum is considered to have two sister phyla: Chlamydiota (formerly Chlamydiae) and Lentisphaerota (formerly Lentisphaerae) within the PVC superphylum, all are encompassed by the present disclosure. In some embodiments, Verrucomicrobiota may comprise Akkermansia muciniphila, or any species or isolate thereof. Still further, “Proteobacteria”, also called Pseudomonadota is a major phylum of Gram-negative bacteria, which includes a wide variety of pathogenic genera, such as Escherichia, Salmonella, Vibrio, Yersinia, Legionella, and many others. “Actinobacteria” also called Actinomycetota are a diverse phylum of Gram-positive bacteria with high G+C content found in soil. “Firmicutes” also called Bacillota are a phylum of bacteria, most of which have gram-positive cell wall structure, and they are all defined as the core group of related forms called the low-G+C group, in contrast to the Actinomycetota. “Tenericutes” or Mycoplasmatota is a phylum of gram-negative bacteria consisting of cells bounded by a plasma membrane, and they are devoid of cell walls. This phylum contains the class Mollicutes. Notable genera that may be applicable in the present disclosure, include Mycoplasma, Spiroplasma, Ureaplasma, and Candidatus Phytoplasma. In more specific embodiments, Bacteroidota bacterium appliable in the disclosed methods may be any bacteria of the genus Bacteroides, Bacteroidia, Bacteroidales, Bacteroidaceae and Phocaeicola, and any combinations thereof.

In more specific embodiments, the bacteriophage administered by the disclosed methods modulates the phenotype of at least one bacteria of the phylum selected from Bacteroidota. In more specific embodiment, such Bacteroidota bacterium is of the genus Bacteroides, Bacteroidia, Bacteroidales, Bacteroidaceae and/or Phocaeicola. “Bacteroides” is a genus of Gram-negative, obligate anaerobic bacteria. Bacteroides species are non endospore-forming bacilli, and their membranes contain sphingolipids, and also meso-diaminopimelic acid in their peptidoglycan layer. Bacteroides species form the most substantial portion of the mammalian gastrointestinal microbiota. In yet some further embodiments, bacteria analyzed for phase variations in the methods of the preset disclosure may comprises at least one of Bacteroides fragilis and Bacteroides thetaiotaomicron, or any isolate or species thereof.

In more specific embodiments, the disclosed methods involve the administration of at least one bacteriophage that modulates the phenotype of a Bacteroidota bacterium, specifically, at least one of Bacteroides fragilis and Bacteroides thetaiotaomicron, or any isolate or species thereof. “Bacteroides fragilis” is an anaerobic, Gram-negative, pleomorphic to rod-shaped bacterium. It belongs to the Bacteroides genus, and Bacteroidota phylum. Bacteroides fragilis resides in the human gastrointestinal tract and is essential to healthy gastrointestinal function such as mucosal immunity and host nutrition. B. fragilis utilizes a complex series of surface proteins, lipopolysaccharide chains, and outer membrane vesicles to help survive the volatile intestinal micro-environment. B. fragilis can also modulate its surface by expressing different polysaccharides, particularly, B. fragilis polysaccharide A (PSA) can modulate the host immune system. “Bacteroides thetaiotaomicron” is a gram-negative, rod shaped obligate anaerobic bacterium that is a prominent member of the normal gut microbiome in the distal intestines. The term “isolate” or a “genetic isolate” as used herein refers to a population of organisms with little genetic mixing with other organisms within the same species due to geographic isolation or other factors that prevent reproduction. The term “species” (pl. species) is the basic unit of classification and a taxonomic rank of an organism, defined by their karyotype, DNA sequence, morphology, behavior, or ecological niche.

Still further, in some other embodiments, the Bacteroidota bacterium comprises at least one of Bacteroides uniformis, Bacteroides vulgatus, Bacteroides xylanisolvens, Bacteroides caccae, Bacteroides cellulosilyticus, Bacteroides massiliensis, Bacteroides ovatus, Bacteroides stercoris and Bacteroides faecis or any isolate or species thereof.

In some embodiments the bacteriophage administered by the disclosed methods modulates, or is associated with modulation of, phase variation in at least one gene locus in the bacteria. More specifically, modulation of phase variation in accordance with the present disclosure comprise phase variation/s in at least one intergenic region/s and/or intragenic region/s of the locus.

“Intergenic regions” are a stretch of DNA sequences located between genes, and include promoters, enhancers, and other regulatory elements, origins of replication, transposons and viruses. Non-functional DNA elements include for example pseudogenes and repetitive DNA, both of which are types of junk DNA. “Intergenic regions” are a stretch of DNA sequences located within genes.

In some specific embodiments, the phase variation caused by, or associated with, the bacteriophage used in the disclosed methods may occur at any locus. In more specific embodiments, the at least one locus may comprise nucleic acid sequence/s encoding and/or regulating at least one outersurface and/or internal molecule and/or at least one molecule that modify or regulate said at least one outersurface and/or internal molecule/s in the bacteria.

“Outersurface” molecules refer herein to molecules which reside within the cell surface of an organism and/or at the outside part of the cell surface of an organism. “Internal” molecules refer herein to molecules which reside inside the cell, in the internal area of an organism's cell. The locus determined for phase variations may comprise, in some embodiments, a sequence that modify or regulate the outer or inner membrane, specifically, a sequence that changes directly or indirectly the activity, stability, post translational modifications, of the outer or inner molecule, thereby, defining the term “modify” as used herein.

In some embodiments, the at least one locus comprises at least one locus of bacterial outersurface molecules and/or of at least one target recognition component (e.g., specificity protein) of bacterial restriction modification system (R-M). More specifically, the bacterial outersurface molecules comprise at least one of capsular polysaccharide (CPS), polysaccharide utilization loci (PUL), SusC/D, ribosomal RNA (rRNA) 23S, rRNA 16S, fimbria, transposase, HsdS, outer membrane protein A (OmpA), Tetracycline resistance protein (Tet (Q) and Transfer RNA (tRNA), helix-turn-helix transcriptional regulator or DUF6198 family protein, HAD family phosphatase or MATE family efflux transporter, ATP-binding protein, Upx Y family transcription antiterminator, TonB-dependent receptor. In some embodiments, the bacterial outersurface molecules comprise at least one capsular polysaccharide (CPS).

Bacterial “capsular polysaccharides (CPSs)” are a diverse class of high molecular weight polysaccharides, that confer protective effects to their bearers against a wide range of environmental pressures, most notably against the immune system during infection of their animal hosts, by hiding cell-surface components that might otherwise elicit host immune response. Although capsules are often associated with descriptions of pathogenic bacteria due to the large proportion of encapsulated invasive pathogens, non-pathogenic and commensal bacteria also benefit from the ability to envelope themselves with a capsule. In Gram-negative bacteria, capsular polysaccharides are often attached to the outer membrane at their reducing end through covalently-linked lipids that are inserted into the lipid bilayer of the membrane. This provides a surface layer of water-saturated, high molecular weight polysaccharides that limit desiccation in the face of harsh environmental conditions, block infection by most bacteriophages, and thwart phagocytosis and other host immune responses by physically restricting access to cell surface antigens. These polysaccharide cloaks are likely rational targets for wide-spectrum therapeutic compounds aimed at replacing or supplementing antibiotic treatment of microbial infections, as removal of the capsule exposes bacteria to routine immune clearance pathways mediated frequently by activation of the complement system. Different serotypes of capsular polysaccharides express structural differences in the capsular polysaccharide (CPS). One example is the CPS serotype 3 or CPS3). Still further, “Polysaccharide utilization loci (PULs)”, a unique feature of Bacteriodetes genomes, are clusters of colocalized, coregulated genes, the products of which orchestrate the detection, sequestration, enzymatic digestion, and transport of complex carbohydrates. PULs encode a complement of cell surface glycan-binding proteins (SGBPs), TonB-dependent transporters (TBDTs), carbohydrate-active enzymes (CAZymes) (most frequently glycoside hydrolases (GHs,) polysaccharide lyases (PLs) and carbohydrate esterases (CEs) where substrate appropriate), and carbohydrate sensors/transcriptional regulators. PULs also include ancillary enzymes such as proteases, sulfatases, and phosphatases. The most well-studied PUL-encoded glycan-up-take system is the “starch utilization system (Sus)”, which binds, degrades, and imports starch into the cell. This gene cluster of Sus is composed of susRABCDEFG. SusR is an inner membrane-spanning sensor/regulator protein that recognizes maltose, in the periplasm and triggers the rapid upregulation of the sus genes. The outer membrane lipoproteins SusDEF facilitate the binding of starch to the cell surface, and bound starch is then hydrolyzed by the α-amylase SusG. The resulting maltooligosaccharides are shuttled into the periplasm via SusC, a TonB-dependent transporter, and further depolymerized by the neopullulanase SusA and α-glucosidase SusB.

More specifically, “Ribosomal ribonucleic acid (rRNA)” is non-coding RNA forming the primary component of ribosomes. rRNA is a ribozyme involved in protein synthesis in ribosomes. Ribosomal RNA comprises two major ribosomal subunit: the large subunit (LSU) and the small subunit (SSU), forms together a functioning ribosome. In prokaryotes, the LSU and SSU are called the 50S and 30S subunits, respectively. There are three types of rRNA found in prokaryotic ribosomes: “23S rRNA” and 5S rRNA in the LSU and “16S rRNA” in the SSU. “Fimbriae”, also referred to as pili, mediate adhesion of the bacterial cell to host tissue through interaction with receptors located on the host cell. A “transposase”, as used herein, is any of a class of enzymes capable of binding to the end of a transposon and catalyzing its movement to another part of a genome, typically by a cut-and-paste mechanism or a replicative mechanism, in a process known as transposition. Still further, “Hydroxysteroid dehydrogenases (HSDs)”, are a group of alcohol oxidoreductases that catalyze the dehydrogenation of hydroxysteroids. These enzymes also catalyze the reverse reaction, acting as ketosteroid reductases (KSRs). There are several types of HSDs, including 3α-HSD, 3β-HSD, 11β-HSD, 17-HSD and 20β-HSD, applicable in the present disclosure. “Outer membrane protein A (OmpA)” is a multi-functional major outer membrane protein of Escherichia coli and other Enterobacteriaceae family, that also serves as a receptor for several bacteriocin and bacteriophages. Still further, “Tet (Q)” is a tetracycline resistance ribosomal protection protein. Its gene is associated with a conjugative transposon and has been found in both Gram-positive and Gram-negative bacteria. “Transfer RNA” (abbreviated “tRNA”) is an adaptor molecule composed of RNA, that serves as the physical link between the mRNA and the amino acid sequence of proteins. “Helix-turn-helix” is a DNA-binding protein (DBP). The helix-turn-helix (HTH) is a major structural motif capable of binding DNA. Each monomer incorporates two a helices, joined by a short strand of amino acids, that bind to the major groove of DNA. The HTH motif occurs in many proteins that regulate gene expression. The “DUF6198 family protein” family, as used herein, represents a putative integral membrane protein that is likely to be the membrane component of an ABC transport (ATP-binding cassette transport) system. The ABC system utilize the energy of adenosine triphosphate (ATP) binding and hydrolysis to provide the energy needed for the translocation of substrates across membranes, either for uptake or for export of a substrate. “Phosphatases of the haloacid dehalogenase (HAD)” superfamily of hydrolases are a very large class of enzymes that have evolved to dephosphorylate substrates with often exquisite specificities. The “MATE” (“Multidrug And Toxic Compound Extrusion”) efflux transport family, as used herein, is one among five multidrug efflux transporter families, known to play an important role in intrinsic and acquired resistance in many bacteria. MATE is a family of proteins which function as drug/sodium or proton antiporters. Still further, “ATP-binding proteins” are proteins which possess an ATP-binding site and are capable of binding ATP. ATP binding sites, are present in many proteins including active membrane transporters, microtubule subunits, flagellum proteins, and various hydrolytic and proteolytic enzymes, all are encompassed by the present disclosure. “UpxY family transcription antiterminator” are involved in the biosynthesis of Bacteroides fragilis polysaccharides. A single strain of Bacteroides fragilis synthesizes eight distinct capsular polysaccharides, designated PSA to PSH, and encoded by eight separate polysaccharide biosynthesis loci. “TonB-dependent receptors”, as used herein, also known as outer membrane receptors, are a family of beta barrel proteins named for their localization in the outer membrane of gram-negative bacteria. TonB complexes sense signals from the outside of bacterial cells and transmit them into the cytoplasm, leading to transcriptional activation of target genes.

Still further, in some embodiments, the at least one bacteriophage used in the disclosed methods modulates or is associated with modulation of phase variation in the CPS locus. In more specific embodiments, the CPS comprise three to ten different CPS operons, leading to the production of distinct capsule structures or polysaccharides (PS). In yet some further embodiments, the PS comprise polysaccharides A to H (PSA, PSB, PSC, PSD, PSE, PSH) of distinct structures.

“Polysaccharides (PS)” are long-chain polymeric carbohydrates composed of monosaccharide units bound together by glycosidic linkages. Among them, D-glucose is the most predominant compound. In addition to glucose, some other derivatives of monosaccharides are also found in polysaccharides that include simple sugar acids such as glucuronic and iduronic acid, amino sugars like D-galactosamine, D-glucosamine, and their derivatives like N-acetylneuraminic acid and N-acetylmuramic acid. Microbial polysaccharides are produced by microorganisms such as bacteria, fungi, yeast, and algae. These polysaccharides include carbohydrates that are produced and accumulated inside the cells such as glycogen where they function as energy and carbon reserves. Others include polysaccharides of cell wall such as chitin, involved in stabilization of the integrity of the cells, and extracellular polysaccharides secreted by the cells. The later type either form a capsule (capsular polysaccharides) that remains associated with the surface of the cell, or a slime loosely attached to the surface of the cell (exopolysaccharides). Besides their function as structural material, polysaccharides are also considered important functional material, playing a variety of roles in many physiological and biological processes. They display anti-tumor, anti-hyperglycemic, and immune modulatory potentials.

“Polysaccharide A (PSA)” is a capsular carbohydrate from the commensal gut bacteria Bacteroides fragilis. PSA exhibits unique immunomodulatory effects that seem to come from the zwitterionic nature of the sugar. PSA is one of the two main polysaccharides (the other is “polysaccharide B (PSB)”) composing a capsular polysaccharide complex, which Bacteroides fragilis produces and encases itself with.

“Polysaccharide F (PSF)” is a type of long chain sugar molecule produced by some bacteria. These complex carbohydrates are found on the outer layer of the bacterial cell wall and contribute to the overall structure and function of the cell. PSF was suggested to play a role in immune evasion, by interacting with the host's immune system, helping bacteria to avoid being destroyed by immune cells.

“Capsular polysaccharide serotype 3 (CPS3)” refers to a complex carbohydrate (sugar) molecule with a unique structure and composition compared to other CPS serotypes that is recognized by the immune system as “serotype 3”. Serotyping is a technique used to classify bacteria based on the specific antigens (molecules the immune system recognizes) present on their surface. Still further, in some embodiments of the disclosed methods, phase variations comprise at least one inversion in at least one promoter region of at least one gene residing in the PSA and/or CPS3 and/or PSF loci, thereby converting the ON/OFF orientation of the at least one promoter region/s. In some embodiments, an inverted orientation of at least one promoter region results in an OFF status of the promoter, thereby reduces the expression levels of at least one protein product regulated by the promoter. Non-limiting embodiments for such products include the PSA protein which is regulated by the PSA promoter and the PSB promoter which is regulated by the S protein of the bacterial restriction modification system (R-M).

In yet some further embodiments, the phase variations caused by the at least one bacteriophage administered by the disclosed methods, comprise at least one inversion in at least one gene of at least one target recognition component of at least one bacterial restriction modification system (R-M) thereby generating a specific combination of the gene to encode one of the target recognition components of the bacteria, having a distinct recognition site.

The specificity protein referred herein is of the Type I restriction modification system (R-M). The modular nature of Type I specificity proteins allows the protein to change its recognition site by varying the N- and C-terminal target recognition domains (TRD). Reversible DNA inversions within the specificity protein locus of leads to the generation of eight specificity proteins with distinct recognition sites. By creating mutants, each able to produce only one specificity protein from this region.

In more specific embodiments, the restriction modification system is a type I restriction modification (Type1 R-M) system. Accordingly, the at least one target recognition component of the Type1 R-M system is or comprises at least one specificity protein.

In some specific embodiments, the Type1 R-M system used herein is of B. fragilis. According to some embodiments, the gene region that encodes the specificity protein of the Type1 R-M system of B. fragilis, comprises several gene fragments indicated herein as head (H) and tail (T) fragments, specifically, two head fragments (indicated herein as 57H and 60H), and four tail fragments (indicated herein as 57T, 58T, 59T and 60T), that may be combined via inversions to create eight different specificity protein, each having a particular specificity to a distinct recognition sequence (e.g., within a bacteriophage, or any invading nucleic acid sequence). In some specific embodiments, the 57H region of the Type1 R-M system of B. fragilis comprises the nucleic acid sequence as denoted by SEQ ID NO: 14. Still further, in some embodiments the 60H region of the Type1 R-M system of B. fragilis comprises the nucleic acid sequence as denoted by SEQ ID NO: 23. In yet some further embodiments, the 57T region of the Type1 R-M system of B. fragilis comprises the nucleic acid sequence as denoted by SEQ ID NO: 15, and SEQ ID NO: 24. In some further embodiments, the 58T region of the Type1 R-M system of B. fragilis comprises the nucleic acid sequence as denoted by SEQ ID NO: 17. In further embodiments, the 59T region of the Type 1 R-M system of B. fragilis comprises the nucleic acid sequence as denoted by SEQ ID NO: 19. Still further, in some embodiments, the 60T region of the Type1 R-M system of B. fragilis comprises the nucleic acid sequence as denoted by SEQ ID NO: 21. Accordingly, upon various inversions, the following combined orientations of the head and tail gene fragments, may create eight different combinations, each encoding to a distinct specificity protein, that display specificity to a particular target recognition sequence within a foreign nucleic acid sequence (e.g., bacteriophage). In some embodiments, the following eight various gene combination may encode eight different specificity proteins. More specifically, the 57H/57T combination comprises the nucleic acid sequence of SEQ ID NO: 13, and encodes a specificity protein designated herein as 57/57. In yet some further embodiments, the 57H/58T combination comprises the nucleic acid sequence of SEQ ID NO: 16, and encodes a specificity protein designated herein as 57/58. Still further, in some embodiments, the 57H/59T combination comprises the nucleic acid sequence of SEQ ID NO: 18, and encodes a specificity protein designated herein as 57/59. In some further embodiments, the 57H/60T combination comprises the nucleic acid sequence of SEQ ID NO: 20, and encodes a specificity protein designated herein as 57/60. More specifically, the 60H/57T combination comprises the nucleic acid sequence of SEQ ID NO: 22, and encodes a specificity protein designated herein as 60/57. In yet some further embodiments, the 60H/58T combination comprises the nucleic acid sequence of SEQ ID NO: 25, and encodes a specificity protein designated herein as 60/58. Still further, in some embodiments, the 60H/59T combination comprises the nucleic acid sequence of SEQ ID NO: 26, and encodes a specificity protein designated herein as 60/59. In some further embodiments, the 60H/60T combination comprises the nucleic acid sequence of SEQ ID NO: 27, and encodes a specificity protein designated herein as 60/60.

In some embodiments, phase variations in the CPS loci and/or the Type 1 R-M system locus, involve DNA inversion. More specifically, this term refers to a genetic event where a segment of DNA within a chromosome is flipped in orientation relative to its surrounding sequence. In other words, a segment of DNA undergoes a 180-degree rotation around its axis, resulting in the inversion of the s orientation of the sequence.

DNA inversions can occur naturally as a result of genetic recombination processes such as homologous recombination or site-specific recombination. DNA inversion may lead to at least one of alteration of gene expression patterns (disruption of regulatory elements or change the spatial arrangement of genes relative to regulatory sequences, leading to changes in gene expression levels or patterns). DNA inversions may further involve disruption of coding sequences. Specifically, alteration of the reading frame of protein-coding genes, potentially leading to the production of non-functional or altered proteins. Inversions can in some embodiments generate new genetic variants by reshuffling existing genetic material within a chromosome.

Still further, in some embodiments, the at least one bacteriophage administered by the disclosed methods modulates or is associated with modulation of the phenotype of at least one bacteria in the subject. According to certain embodiments, the modulated phenotype is the expression of bacterial outersurface molecules and/or of at least one specificity protein of bacterial restriction modification system (R-M)]. In some embodiments where the modulated phenotype relates to bacterial outersurface molecules, such molecules may comprise at least one of PSA, PSF, PSB and/or CPS3. CPS1, CPS5, CPS6, PSH, and any other outersurface protein.

In some embodiments, the bacteriophage used by the disclosed methods may cause or be associated with phase variations in both loci, specifically, PSA and/or CPS3 and/or PSF loci and the restriction modification system (R-M) locus. In some embodiments, the phase variation occur together in these loci and optionally, in other loci in at least one bacteria.

It should be appreciated that in some embodiments, the at least one bacteriophage useful in the methods of the present disclosure, may be a natural bacteriophage. In yet some further embodiments, the bacteriophage used in the disclosed methods may be at least one engineered bacteriophage. In some further embodiments, the disclosed methods may use engineered together with natural bacteriophages for immuno modulating the subject (or modulating an immune response in the subject). A “natural bacteriophage” refers to a phage isolated from nature and not subjected to artificial genetic engineering. An “engineered bacteriophage” refers to a phage which was generated or subjected to genetic engineering. “Genetic engineering” as used herein refers to the known standard technologies for the modification and manipulation of the genetic makeup of cells, including the transfer of genes within and across species boundaries to produce improved or novel cells and/or organisms. An organism that is generated through genetic engineering is considered to be genetically modified (GM) and the resulting entity is a genetically modified organism (GMO).

In some embodiments, the disclosed methods may involve and comprise the step of administering to the subject at least one bacteriophage belonging to the siphoviridae family of the Caudovirales order. In some specific and non-limiting embodiments, such at least one bacteriophage comprises at least one of: the bacteriophage as denoted by any one of: OTU 0791, OTU 0820, OTU 1202, OTU 0165, OTU 1130, OTU 1490 of the IBDMDB database, and bacteriophage comprising the nucleic acid sequence as denoted by SEQ ID NO: 28, also designated herein as Barc2635.

In some specific embodiments, the disclosed methods comprise the step of administering an effective amount of the Barc2635 bacteriophage, thereby modulating the phenotype of bacteria in the host.

“Siphoviridae” is a family of double-stranded DNA viruses in the order Caudovirales. The characteristic structural features of this family are a nonenveloped head and noncontractile tail. Caudoviricetes is a class of viruses known as the tailed bacteriophages (cauda is Latin for “tail”). Under the Baltimore classification scheme, the Caudoviricetes are group I viruses as they have double stranded DNA (dsDNA) genomes, which can be anywhere from 18,000 base pairs to 500,000 base pairs in length. The virus particles have a distinct shape; each virion has an icosahedral head that contains the viral genome, and is attached to a flexible tail by a connector protein.

In yet some further embodiments, the disclosed methods may use engineered bacteriophage/s. In some embodiments, such engineered bacteriophage comprises at least one exogeneous recognition sequence of at least one bacterial restriction modification system (R-M). The term “exogeneous| refers herein to a feature which has an external origin and is not a natural integral part of the bacteriophage.

The at least one recognition sequence is specifically recognized by at least one component of the restriction modification system of at least one bacterium displaying a desired phenotype. The recognition sequence comprises a 5′ recognition site and a 3′ recognition site separated by 3 to 10 unspecified intervening nucleotides.

In some embodiments, the recognition sequence in the engineered bacteriophage may comprise a 5′ recognition site comprising the nucleic acid sequence GAC. In yet some further embodiments, the 3′ recognition site may comprise any one of GRTY, CTG, TCC, TGC, wherein A is adenine, G is guanin, C is cytosine, T is thymine, R is adenine or guanin, Y is Thymine or cytosine, and N is any nucleic acid residue.

Thus, in some embodiments, the engineered bacteriophage may comprise at least one repeat of the recognition sequence GACNNNNNGRTY (GACN₅GRTY) and/or the reverse complement thereof, specifically, RAYCNNNNNGTC (RAYCN₅GTC), wherein N can be any nucleotide (T, A, C, G), R, can be A or G, and Y can be C or T.

In some embodiments, the engineered bacteriophage may comprise at least one repeat of the recognition sequence GACNNNNNGATC (GACN₅GATC) and/or the reverse complement thereof, specifically, GATCNNNNNGTC (GATCN₅GTC), wherein N can be any nucleotide (T, A, C, G). Still further, in some embodiments, the engineered bacteriophage may comprise at least one repeat of the recognition sequence GACNNNNNNGATC (GACN₆GATC), and/or the reverse complement thereof.

In some embodiments, the engineered bacteriophage may comprise at least one repeat of the recognition sequence GACNNNNNNNGATC (GACN₇GATC), and/or the reverse complement thereof. In some embodiments, the engineered bacteriophage may comprise at least one repeat of the recognition sequence GACNNNNNNNNGATC (GACN₈GATC), and/or the reverse complement thereof. In some embodiments, the engineered bacteriophage may comprise at least one repeat of the recognition sequence GACNNNNNNNNNGATC (GACN₉GATC), and/or the reverse complement thereof. In some embodiments, the engineered bacteriophage may comprise at least one repeat of the recognition sequence GACNNNNNNNNNNGATC (GACN₁₀GATC), and/or the reverse complement thereof. In some embodiments, these recognition sequences are recognized by a B. fragilis S-protein having the 57/59 orientation. In some embodiments, such S-protein of the 57/59 orientation is encoded by the nucleic acid sequence as denoted by SEQ ID NO: 18.

In yet some further alternative embodiments, the engineered bacteriophage may comprise at least one repeat of the recognition sequence GACNNNNNCTG (GACN₅CTG), and/or at least one repeat of the reverse complement thereof, specifically, CAGNNNNNGTC (CAGN₅GTC), wherein N is any nucleotide (T, A, C, G). Still further, in some embodiments, the engineered bacteriophage may comprise at least one repeat of the recognition sequence GACNNNNNNCTG (GACN₇CTG), and/or the reverse complement thereof. In some embodiments, the engineered bacteriophage may comprise at least one repeat of the recognition sequence GACNNNNNNNCTG (GACN₇CTG), and/or the reverse complement thereof. In some embodiments, the engineered bacteriophage may comprise at least one repeat of the recognition sequence GACNNNNNNNNCTG (GACN₈CTG), and/or the reverse complement thereof. In some embodiments, the engineered bacteriophage may comprise at least one repeat of the recognition sequence GACNNNNNNNNNCTG (GACN₉CTG), and/or the reverse complement thereof. In some embodiments, the engineered bacteriophage may comprise at least one repeat of the recognition sequence GACNNNNNNNNNNCTG (GACN₁₀CTG), and/or the reverse complement thereof.

In some embodiments, these recognition sequences are recognized by a B. fragilis S-protein having the 57/58 orientation. In some embodiments, such S-protein of the 57/58 orientation is encoded by the nucleic acid sequence as denoted by SEQ ID NO: 16.

In some alternative embodiments, the engineered bacteriophage may comprise at least one repeat of the recognition sequence GACNNNNNNTCC (GACN₆TCC), and/or the reverse complement thereof.

In some embodiments, these recognition sequences are recognized by a B. fragilis S-protein having the 57/57 orientation. In some embodiments, such S-protein of the 57/57 orientation is encoded by the nucleic acid sequence as denoted by SEQ ID NO: 13.

In some embodiments, the engineered bacteriophage may comprise at least one repeat of the recognition sequence GACNNNNNNTGC (GACN₆TGC), and/or the reverse complement thereof. In some embodiments, these recognition sequences are recognized by a B. fragilis S-protein having the 57/60 orientation. In some embodiments, such S-protein of the 57/60 orientation is encoded by the nucleic acid sequence as denoted by SEQ ID NO: 20.

In some embodiments, modulating the immune response of a subject in accordance with the disclosed methods comprises modulating the levels of at least one of T regulatory cells and/or at least one cytokine in said subject, or any host.

The “regulatory T cells (Tregs)”, or suppressor T cells, refers to a subpopulation of T cells that modulate the immune system, maintain tolerance to self-antigens, and prevent autoimmune disease. T_{reg} cells are immunosuppressive and generally suppress or downregulate induction and proliferation of effector T cells. T_{reg} cells express the biomarkers CD4, FOXP3, and CD25 and are thought to be derived from the same lineage as naïve CD4+ cells.

“Cytokine” refers herein to a broad category of small proteins (~5-25 kDa) important in cell signaling. Cytokines typically exert their functions by interacting with specific cytokine receptors on the target cell surface. Cytokines have been shown to be involved in autocrine, paracrine and endocrine signaling as immunomodulating agents. Cytokines include chemokines, interferons, interleukins, lymphokines, and tumour necrosis factors, but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

In some embodiments, the modulation occurs in a subject. In more specific embodiments, the subject is suffering of at least one immune-related disorder.

In yet some further embodiments, the administered subject may be a subject suffering of an immune-related disorder. In some embodiments, the immune-related disorder is at least one of: a proliferative disorder, an inflammatory disorder, an autoimmune disorder, an immune-deficiency condition and/or an infectious disease. a neurodegenerative and/or cognitive and/or mental disorder, a metabolic disorder, and a condition involving at least one wound in at least one tissue and/or organ of said subject. An “Immune-related disorder” or “Immune-mediated disorder”, as used herein encompasses any condition that is associated with the immune system of a subject, more specifically through inhibition of the immune system, or that can be treated, prevented, or ameliorated by reducing degradation of a certain component of the immune response in a subject, such as the adaptive or innate immune response. An immune-related disorder may include infectious condition (e.g., by a pathogen, specifically, viral, bacterial, or fungal infections), inflammatory disease, autoimmune disorders, immunodeficiency (e.g., primary or a secondary) metabolic disorders and proliferative disorders, specifically, cancer.

In some embodiments, the pathologic disorder applicable in the methods of the present disclosure may be any proliferative disorder. As used herein to describe the present disclosure, “proliferative disorder”, “malignant neoplastic disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. Malignancy, as contemplated in the present disclosure may be any one of melanomas, carcinomas, lymphomas, leukemia, myeloma, and sarcomas.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges.

It should be understood that the methods of the present disclosure are applicable for any type and/or stage and/or grade of any of the malignant disorders discussed herein or any metastasis thereof. Still further, it must be appreciated that the methods of the present disclosure may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. More specifically, further malignancies that may find utility in the present disclosure can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The present disclosure may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

In some embodiments, the immune-related disorder applicable in the methods of the present disclosure may be an inflammatory disease. The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause. In some embodiments, an inflammatory disease that may be applicable for the methods of the present disclosure may be inflammatory bowel disease (IBD). An “autoimmune disorder” is state in which the immune system gets directed against self-cells or tissues. Autoimmune disorders include for example, but not limited to inflammatory bowel disease (IBD), ulcerative colitis (UC), Crohn's disease (CD), Systemic Lupus Erythematosus (SLE), Rheumatoid Arthritis (RA), fatty liver disease, Lymphocytic colitis, Ischaemic colitis, Diversion colitis, Behçet's syndrome, Indeterminate colitis, Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

More specifically, in some embodiments, the immune-related disorder applicable in the methods of the present disclosure may be an inflammatory bowel disease (IBD). “Inflammatory bowel disease (IBD)” is characterized by repetitive episodes of inflammation of the gastrointestinal tract caused by an abnormal immune response to gut microflora. Inflammatory bowel disease encompasses two types of idiopathic intestinal disease that are differentiated by their location and depth of involvement in the bowel wall. Ulcerative colitis (UC) involves diffuse inflammation of the colonic mucosa. Most often UC affects the rectum (proctitis), but it may extend into the sigmoid (proctosigmoiditis), beyond the sigmoid (distal ulcerative colitis), or include the entire colon up to the cecum (pancolitis). Crohn disease (CD) results in transmural ulceration of any portion of the gastrointestinal tract (GI) most often affecting the terminal ileum and colon. Both diseases are classified by extent (mild, moderate, or severe) and location. CD also is classified by phenotype-inflammatory, stricturing, or penetrating. Besides the GI tract, both Crohn disease and ulcerative colitis have many extraintestinal manifestations. While in most patients, the disorders can be distinguished, in at least 10% of patients, the features are so similar that it is not possible to initially differentiate between the two disorders. Both disorders have a genetic predisposition; neither is curable, and they both carry enormous morbidity. Finally, both increase the risk of colorectal cancer.

In some embodiments, the immune-related disorder applicable in the methods of the present disclosure may be immunodeficiency. “Immunodeficiency” (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer 37immune-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins. Most primary immune-deficiencies are genetic disorders; the majority is diagnosed in children under the age of one, although milder forms may not be recognized until adulthood. While there are over 100 recognized PIDs, most are very rare. Secondary immunodeficiencies occur when the immune system is compromised due to environmental factors. Such factors include but are not limited to chemotherapy, radiotherapy, biological therapy, bone marrow transplantation, gene therapy, adoptive cell transfer or any combinations thereof.

In some embodiments, the immune-related disorder applicable in the methods of the present disclosure may be at least one infectious disease. An “infectious disease” as used herein encompasses any infectious disease caused by a pathogenic agent, specifically, a pathogen. More specifically, such infectious disease may be any pathological disorder caused by a pathogen. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts, or a nematode, as well as toxins and venoms.

In some embodiments, the pathologic disorder applicable in the methods of the present disclosure may be at least one neurodegenerative disorder. “Neurodegeneration” is the umbrella term for the progressive loss of structure or function of neurons, including synaptic dysfunction and death of neurons. Many neurodegenerative diseases including Parkinson's and Alzheimer's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present disclosure. More specifically, the term “neurodegenerative diseases” is the general term for the progressive loss of structure or function of neurons, leading to their death. The major risk factor for neurodegenerative diseases is aging. Mitochondrial DNA mutations as well as oxidative stress both contribute to aging. Many of these diseases are late onset, meaning there is some factors that change as a person ages, for each disease. One constant factor is that in each disease, neurons gradually lose function as the disease progresses with age.

Still further, in some embodiments, the pathologic disorder applicable in the methods of the present disclosure may be any mental condition. A “mental disorder” is characterized by a clinically significant disturbance in an individual's cognition, emotional regulation, or behavior. It is usually associated with distress or impairment in important areas of functioning. Mental disorders may also be referred to as mental health conditions. The latter is a broader term covering mental disorders, psychosocial disabilities and (other) mental states associated with significant distress, impairment in functioning, or risk of self-harm. There are many different types of mental disorders including for example anxiety disorders, depression, bipolar disorder, post-traumatic stress disorder (PTSD), schizophrenia, eating disorders, such as anorexia nervosa and bulimia nervosa, disruptive behavior and dissocial disorders and neurodevelopmental disorders such as autism spectrum disorder (ASD), and attention deficit hyperactivity disorder (ADHD) amongst others. In some embodiments, the pathologic disorder applicable in the methods of the present disclosure may be at least one metabolic disorder. “Metabolic disorders” may include atherosclerosis and peripheral vascular diseases, as well as cardiovascular diseases such as coronary artery diseases (CAD). Of particular interest in connection with metabolic disorders are conditions associated with obesity, hypertension, elevated cholesterol (combined hyperlipidemia), such conditions often termed metabolic syndrome (it is also known as Syndrome X, Reavan's syndrome, or CHAOS). It should be noted that the disclosed conditions may be congenital or acquired conditions. In some embodiments, the immune-related disorder used by the present disclosure refers to a condition involving at least one wound in at least one tissue and/or organ of a subject. A “wound” is any disruption of or damage to living tissue, such as skin, mucous membranes, or organs. Wounds can either be the sudden result of direct trauma (mechanical, thermal, chemical), or can develop slowly over time due to underlying disease processes such as diabetes mellitus, venous/arterial insufficiency, or immunologic disease. In certain specific embodiments, the disclosed method may be applicable for inflammatory conditions such as inflammatory bowel disease (IBD).

In some particular and specific embodiments, the administered subject may be (i) a subject suffering of at least one proliferative disorder. In yet some further embodiments (ii), the modulation of the immune response and/or immune-system, and/or the immunological state of the subject comprises reduction in T regulatory cells (Tregs). In some embodiments, the reduction in T regs is caused in response to administration of the bacteriophage and may be thus either be associated with the bacteriophage, and/or caused directly or indirectly by the administered bacteriophage. In some embodiments, the observed effect on the T regs levels may be a result of the bacteriophage action on at least one bacteria in the subject, specifically, bacteria residing in the gut microbiome. In some further embodiments, (iii), the bacteria comprise the gut bacteria B. fragilis. In some additional embodiments (iv), the desired phenotype of the bacteria comprises reduced expression of PSA and/or increased expression of PSF. In some embodiments, the reduced expression of PSA and/or the increased expression of PSF is caused in response to administration of the bacteriophage and may therefore in some embodiments be a direct or indirect result of the bacteriophage presence. Thus, according to specific embodiments, the subject is suffering of at least one proliferative disorder (specifically, cancer), the immune-modulation comprises reduction in Tregs in the subject, and the bacteria is B. fragilis, showing reduced expression of PSA and/or increased expression of PSF, in response to administration of the phage. In some embodiments, the reduction in Tregs in accordance with some embodiments encompasses any reduction of about 1% to about 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more reduction in Tregs, as compared to the levels, number, activity or amount of the Tregs without or before the administration of the bacteriophage. In some specific embodiments, where the cancer is colorectal cancer, the reduction can be in colorectal T regs.

In yet some further embodiments, the reduction in the expression of the PSA in accordance with some embodiments encompasses any reduction of about 1% to about 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more reduction in expression of the PSA, as compared to the levels of the expression of the PSA without or before the administration of the bacteriophage.

In more specific embodiments, for a subject as indicated above (suffering of at least one proliferative disorder), the method comprises the administration of the following bacteriophage/s, specifically, at least one bacteriophage as denoted by any one of: OTU 0791, OTU 0820, OTU 1202, of the IBDMDB database, and/or bacteriophage comprising the nucleic acid sequence as denoted by SEQ ID NO: 28, specifically, the bacteriophage designated herein Barc2635.

In some other more specific embodiments, the method comprises the administration of the following bacteriophage/s which reduces the expression of PSA specifically, at least one bacteriophage as denoted by any one of: OTU 0791, OTU 0820, OTU 1202, OTU 0576, OTU 0584, OTU 1146, OTU 1125, and OTU 0802 of the IBDMDB database. In other embodiments, these bacteriophages reduce the expression of PSA in B. fragilis.

In some other embodiments, the method comprises the administration of the following bacteriophage/s which increases the expression of PSA specifically, at least one bacteriophage as denoted by any one of: OTU 0031 or viral OTU 0005 of the IBDMDB database. In other embodiments, these bacteriophages increase the expression of PSA in B. fragilis.

In other embodiments, the method comprises the administration of the following bacteriophage/s which increases the expression of CPS specifically, at least one bacteriophage as denoted by any one of: OUT 0165, OUT 1130, OUT 1490, OTU 0115, OTU 0873, OTU 1100, and OTU 1014 of the IBDMDB database.

In yet some alternative or additional embodiments, the disclosed methods may use a bacteriophage that is an engineered bacteriophage comprising at least one exogeneous recognition sequence of type 1 R-M system of B. fragilis specifically recognized by a specific S protein of encoded by the HsdS genes of said B. fragilis. The recognition sequence comprises a 5′ recognition site sequence of: GAC; and a 3′ recognition site selected from: GRTY, CTG, TCC and TGC; and/or the reverse complement thereof. The 5′ recognition site and a 3′ recognition site are separated by 3 to 10 intervening N nucleotides. It should be noted that A is adenine, G is guanin, C is cytosine, T is thymine, R is adenine (A) or guanin (G), Y is cytosine (C) or thymine (T), and N is any nucleic acid residue.

In yet some further embodiments, the engineered bacteriophage used in the disclosed methods comprises at least one repeat of a recognition sequence comprising: (a), the sequence comprising GACNNNNNGATC, and/or the reverse complement thereof comprising the sequence GATCNNNNNGTC; and/or (b), the sequence comprising GACNNNNNCTG, and/or the reverse complement thereof comprising the sequence CAGNNNNNGTC. It should be understood that A is adenine, G is guanin, C is cytosine, T is thymine, R is A or G, Y is C or T, and N is any nucleic acid residue.

In some embodiments, the engineered bacteriophage used in the disclosed methods may comprises an exogeneous nucleic acid sequence as denoted by SEQ ID NO: 10, comprising at least one of recognition sequence, specifically, the recognition sequence GACNNNNNGATC, and its reverse complement of GATCNNNNNGTC. In some embodiments, this recognition sequence is recognized by a specificity protein encoded by the bacterial (B. fragilis) genomic 57H/59T, regions, that comprise the nucleic acid sequence as denoted by SEQ ID NO: 18. In yet some further alternative or additional embodiments, the engineered bacteriophage used in the disclosed methods may comprises an exogeneous nucleic acid sequence as denoted by SEQ ID NO: 12, comprising at least one of recognition sequence, specifically, the recognition sequence GACNNNNNCTG, and its reverse complement of CAGNNNNNGTC. In some embodiments, this recognition sequence is recognized by a specificity protein encoded by the bacterial (B. fragilis) genomic 57H/58T, regions, that comprise the nucleic acid sequence as denoted by SEQ ID NO: 16. In some embodiments, the method is specifically applicable for a subject suffering of a colorectal cancer (CRC).

“Colorectal cancer (CRC)”, or bowel cancer or colon cancer, or rectal cancer, refers to the development of cancer from the colon or rectum (parts of the large intestine). Signs and symptoms may include blood in the stool, a change in bowel movements, weight loss, abdominal pain and fatigue.

In some embodiments, (i) the subject is suffering of at least one inflammatory condition; (ii) the modulation of the immune system response/state of the subject comprises increase in T regulatory cells (Tregs); (iii) the microorganism is the gut bacteria B. thetaiotaomicron; and (iv) the desired phenotype of said bacteria comprises increased expression of CPS3.

According to such embodiments, the bacteriophage useful according to these embodiments, may be at least one of: bacteriophage as denoted by any one of: OTU 0165, OTU 1130, OTU 1490, of the IBDMDB database.

In yet some further embodiments, such method may be applicable for subjects suffering of an inflammatory condition, for example, IBD (UC, CD).

A further aspect of the present disclosure relates to a method for treating, preventing, inhibiting, reducing, eliminating, protecting or delaying the onset of at least one immune-related disorder in a subject The method comprising the steps of administering to the subject a therapeutically effective amount of at least one bacteriophage or any vehicle, matrix, nano- or micro-particle comprising the same, and/or any composition thereof. In some embodiments, the bacteriophage used in the disclosed method may be characterized by at least one of the following features. In some embodiments (i), the bacteriophage modulates, or is associated with modulation of, phase variation of at least one locus (e.g., gene locus) of the at least one bacteria in the subject. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject.

More specifically, in some embodiments, the disclosed methods comprise the step of administering to a subject an effective amount of at least one bacteriophage that modulates, or is associated with modulation of, phase variation of at least one locus (e.g., gene locus) of the at least one bacteria in the subject/host. In yet some other embodiments, the disclosed methods comprise the step of administering to a subject an effective amount of at least one bacteriophage that comprises at least one recognition sequence for at least one bacterial restriction modification system (R-M). The recognition sequence in the bacteriophage is specifically recognized by at least one component of the R-M system of at least one bacterium displaying a desired phenotype and/or a genotype associated with the desired phenotype. Still further, in some embodiments, the disclosed methods comprise the step of administering to a subject an effective amount of at least one bacteriophage that modulates bacterial functionality in the subject. Still further, in some embodiments, the disclosed methods comprise the step of administering to a subject an effective amount of at least one bacteriophage that modulates, or is associated with modulation of, phase variation of at least one locus of the at least one bacteria in the subject, the phage comprises at least one recognition sequence for at least one bacterial restriction modification system (R-M), and the phage modulates bacterial functionality in the subject. In some embodiments, the disclosed methods comprise the step of administering to a subject an effective amount of at least one bacteriophage that modulates, or is associated with modulation of, phase variation of at least one locus of the at least one bacteria in the subject, and that comprises at least one recognition sequence for at least one bacterial restriction modification system (R-M). Still further, in some embodiments, the disclosed methods comprise the step of administering to a subject an effective amount of at least one bacteriophage that modulates, or is associated with modulation of, phase variation of at least one locus of the at least one bacteria in the subject, and that modulates bacterial functionality in the subject.

In some embodiments, the disclosed methods comprise the step of administering to a subject an effective amount of at least one bacteriophage that comprises at least one recognition sequence for at least one bacterial restriction modification system (R-M), and that modulates bacterial functionality in the subject.

In some embodiments, the bacteriophage useful in the disclosed methods comprises at least one recognition sequence for at least one bacterial restriction modification system (R-M). In some embodiments, the recognition sequence comprises a 5′ recognition site and a 3′ recognition site separated by 3 to 10 unspecified intervening nucleotides.

The term “treatment” or “prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the methods according to the present disclosure, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the present disclosure described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the present disclosure.

As used herein, the term “therapeutically effective amount” means an amount of a compound or composition which is administered to a subject in need thereof, necessary to effect a beneficial change in the severity of a disease or disorder, or prevent such disease, in said subject. This amount should also be within specific pharmacological ranges, to avoid toxic effects by over-dosing. For example, in the present invention, a therapeutically effective amount of at least one of the bacteriophages of the invention, for the treatment of an immune-related disorder would be the amount of these phages administered to a subject which would induce a beneficial change in the subject, alleviating, ameliorating, or preventing the recurrence of said immune-related disorder, without causing detrimental side effects, or causing only mild side-effects. It is understood that the therapeutically effective amount is not an absolute term and depends on subjective circumstances, such as the subject's age, health, weight, and various other statistics, as described in the and specifically determined by the attendant physician or other person skilled in the art after an evaluation of the subject's conditions and requirements.

It should be noted that the bacteriophages of the present disclosure, either native, engineered, or any combination or cocktail thereof, as well as any compositions thereof as described herein may be presented in unit dose forms containing a predetermined amount of each active ingredient per dose. Such a unit may be adapted to provide 0.1×10⁸ to 1×10¹², plaque forming units (PFU) of the disclosed bacteriophage, per Kg of body weight of the administered subject. Specifically, either 0.1−10×10⁸ PFU/Kg, 0.5−10×10⁸ PFU/Kg, 1−10×10⁸ PFU/Kg, 5−10×10⁸ PFU/Kg, 10×10⁸ PFU/Kg, or 1×10⁹ PFU/Kg, or more specifically, 5×10⁹ PFU/Kg to 1×10¹² PFU/Kg. More specifically, said effective dosage is about 5×10⁹ PFU/Kg to 0.5×10¹² PFU/Kg of the bacteriophage, 5×10⁹ PFU/Kg to 1×10¹¹ PFU/Kg, about 5×10⁹ PFU/Kg to 0.5×10¹¹ PFU/Kg, about 5×10⁹ PFU/Kg to 5×10¹⁰ PFU/Kg, specifically, about 5×10⁹−5×10¹⁰ PFU/Kg. Such doses can be provided in a single dose or as a number of discrete doses. In case a single dose may be administered, a dosage unit form may comprise an amount of about 5×10⁹ PFU/Kg to about 5×10¹⁰ PFU/Kg, that may be administered one a day, a week or a month. The ultimate dose will of course depend on the condition being treated, the route of administration and the age, weight and condition of the patient and will be at the doctor's discretion.

It should be further noted that for the method of treatment and prevention provided in the present invention, the therapeutic effective amount, or dosage, is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the bacteriophage used by the invention or any composition of the invention in bodily fluids or tissues. Following successful treatment, it may be desirable to have the patient undergo maintenance therapy to prevent the recurrence of the disease state, wherein the combined composition of the invention is administered in maintenance doses.

It should be appreciated that the bacteriophages of the present disclosure may be effective when administered to an injured subject after 10′, 20′, 30′, 45′, 50′, 60′, 90′, 150′, 180′, 4 hr, 5 hr, 6 hr, 7 hr, 8 hr, 9 hr, 10 hr, 11 hr, 12 hr, 13 hr, 14 hr, 15 hr, 16 hr, 17 hr, 18 hr, 19 hr,20 hr, 21 hr, 22 hr, 23 hr, 24 hr, 2 days, 3 days 4 days, 5 days, 6 days and even 7 days or more after the occurrence of the injury.

In some embodiments of the disclosed methods further comprise a step of administering the at least one bacteria to the subject.

In some embodiments, the disclosed therapeutic methods are applicable for subjects suffering from at least one immune-related disorder, specifically a proliferative disorder, an inflammatory disorder, an autoimmune disorder, an immune-deficiency condition and/or an infectious disease, a neurodegenerative and/or cognitive and/or mental disorder, a metabolic disorder, and a condition involving at least one wound in at least one tissue and/or organ of said subject.

In some embodiments of the disclosed methods, the treatment comprises modulating the immune response of the subject by the methods as defined by the present disclosure as indicated herein above in connection with other aspects of the invention.

In more specific embodiments, the subject is suffering of at least one proliferative disorder. Moreover, according to such embodiment, the administration of the at least one bacteriophage results in the reduction in Tregs in the subject and/or in reduced expression of PSA and/or increased expression of PSF by the gut bacteria B. fragilis in the subject.

In some specific embodiments, the bacteriophage useful for a subject suffering of at least one proliferative disorder, may be at least one of: bacteriophage as denoted by any one of: OTU 0791, OTU 0820, OTU 1202, of the IBDMDB database, and bacteriophage comprising the nucleic acid sequence as denoted by SEQ ID NO: 28, designated herein Barc2635.

In some other more specific embodiments, the method comprises the administration of the following bacteriophage/s which reduces the expression of PSA specifically, at least one bacteriophage as denoted by any one of: OTU 0791, OTU 0820, OTU 1202, OTU 0576, OTU 0584, OTU 1146, OTU 1125, and OTU 0802 of the IBDMDB database. In other embodiments, these bacteriophages reduce the expression of PSA in B. fragilis.

In some other embodiments, the method comprises the administration of the following bacteriophage/s which increases the expression of PSA specifically, at least one bacteriophage as denoted by any one of: OTU 0031 or viral OTU 0005 of the IBDMDB database. In other embodiments, these bacteriophages increase the expression of PSA in B. fragilis.

In yet some alternative or additional embodiments, the bacteriophage useful in the disclosed methods for treating subjects suffering of at least one proliferative disorder is an engineered bacteriophage comprising at least one exogeneous recognition sequence of type 1 R-M system of B. fragilis specifically recognized by a specific S protein of encoded by the HsdS genes of said B. fragilis. The recognition sequence comprises a 5′ recognition site sequence of: GAC; and a 3′ recognition site selected from: GRTY, CTG, TCC and TGC; and/or the reverse complement thereof. The 5′ recognition site and a 3′ recognition site are separated by 3 to 10 intervening N nucleotides. It should be noted that A is adenine, G is guanin, C is cytosine, T is thymine, R is adenine (A) or guanin (G), Y is cytosine (C) or thymine (T), and N is any nucleic acid residue. In yet some further embodiments, the bacteriophage used in the disclosed methods comprises at least one repeat of a recognition sequence comprising: (a), the sequence comprising GACNNNNNGATC, and/or the reverse complement thereof comprising the sequence GATCNNNNNGTC; and/or (b), the sequence comprising GACNNNNNCTG, and/or the reverse complement thereof comprising the sequence CAGNNNNNGTC. It should be understood that A is adenine, G is guanin, C is cytosine, T is thymine, R is A or G, Y is C or T, and N is any nucleic acid residue.

In some embodiments, the bacteriophage comprises at least one recognition sequence comprising the sequence of GACNNNGATC, wherein n is any nucleotide. In some further embodiments, the bacteriophage comprises at least one recognition sequence comprising the sequence of GACNNNNGATC, wherein n is any nucleotide. In some further embodiments, the bacteriophage comprises at least one recognition sequence comprising the sequence of GACNNNNNGATC, wherein n is any nucleotide. In some further embodiments, the bacteriophage comprises at least one recognition sequence comprising the sequence of GACNNNNNNGATC, wherein n is any nucleotide. In some further embodiments, the bacteriophage comprises at least one recognition sequence comprising the sequence of GACNNNNNNNGATC, wherein n is any nucleotide. In some further embodiments, the bacteriophage comprises at least one recognition sequence comprising the sequence of GACNNNNNNNNGATC, wherein n is any nucleotide. In some further embodiments, the bacteriophage comprises at least one recognition sequence comprising the sequence of GACNNNNNNNNNGATC, wherein n is any nucleotide. In some further embodiments, the bacteriophage comprises at least one recognition sequence comprising the sequence of GACNNNNNNNNNNGATC, wherein n is any nucleotide.

In some embodiments, the bacteriophage comprises at least one recognition sequence comprising the sequence of GACNNNCTG, wherein n is any nucleotide. In some further embodiments, the bacteriophage comprises at least one recognition sequence comprising the sequence of GACNNNNCTG, wherein n is any nucleotide. In some further embodiments, the bacteriophage comprises at least one recognition sequence comprising the sequence of GACNNNNNCTG, wherein n is any nucleotide. In some further embodiments, the bacteriophage comprises at least one recognition sequence comprising the sequence of GACNNNNNNCTG, wherein n is any nucleotide. In some further embodiments, the bacteriophage comprises at least one recognition sequence comprising the sequence of GACNNNNNNNCTG, wherein n is any nucleotide. In some further embodiments, the bacteriophage comprises at least one recognition sequence comprising the sequence of GACNNNNNNNNCTG, wherein n is any nucleotide. In some further embodiments, the bacteriophage comprises at least one recognition sequence comprising the sequence of GACNNNNNNNNNCTG, wherein n is any nucleotide. In some further embodiments, the bacteriophage comprises at least one recognition sequence comprising the sequence of GACNNNNNNNNNNCTG, wherein n is any nucleotide.

It should be appreciated that the disclosed methods and uses may apply any of the engineered and/or natural bacteriophages disclosed by the present disclosure and any cocktail, mixture or combination thereof.

In some specific embodiments, the engineered bacteriophage used in the disclosed methods may comprises an exogeneous nucleic acid sequence as denoted by SEQ ID NO: 10, comprising at least one of recognition sequence, specifically, the recognition sequence GACNNNNNGATC, and its reverse complement of GATCNNNNNGTC. In some embodiments, this recognition sequence is recognized by a specificity protein encoded by the bacterial (B. fragilis) genomic 57H/59T, regions, that comprise the nucleic acid sequence as denoted by SEQ ID NO: 18. In yet some further alternative or additional embodiments, the engineered bacteriophage used in the disclosed methods may comprises an exogeneous nucleic acid sequence as denoted by SEQ ID NO: 12, comprising at least one of recognition sequence, specifically, the recognition sequence GACNNNNNCTG, and its reverse complement of CAGNNNNNGTC. In some embodiments, this recognition sequence is recognized by a specificity protein encoded by the bacterial (B. fragilis) genomic 57H/58T, regions, that comprise the nucleic acid sequence as denoted by SEQ ID NO: 16.

In some embodiments, the subject is suffering of a colorectal cancer (CRC). Thus, the therapeutic method is applicable for treating CRC.

A further aspect of the present disclosure relates to a therapeutically effective amount of at least one bacteriophage or any or any vehicle, matrix, nano- or micro-particle comprising the same, and/or any composition thereof, for use in a method for treating, preventing, inhibiting, reducing, eliminating, protecting or delaying the onset of at least one immune-related disorder in a subject. In some embodiments, the bacteriophage used herein may be characterized by at least one of the following features. In some embodiments (i), the bacteriophage modulates, or is associated with modulation of, phase variation of at least one locus (e.g., gene locus) of at least one bacteria in the subject. In yet some further additional or alternative embodiments (ii), the bacteriophage comprises at least one recognition sequence for at least one bacterial restriction modification system (R-M). The recognition sequence in the bacteriophage is specifically recognized by at least one target recognition component of the R-M system of at least one bacterium displaying a desired phenotype and/or a genotype associated with a desired phenotype. In yet some further alternative or additional embodiments (iii), the bacteriophage used in the disclosed methods modulates bacterial functionality in the subject.

A further aspect of the present disclosure relates to an engineered bacteriophage comprising at least one exogeneous recognition sequence of at least one bacterial restriction modification system (R-M) of at least one bacteria. The at least one recognition sequence is specifically recognized by at least one component of the restriction modification system of at least one bacterium displaying a desired phenotype. More specifically, the recognition sequence in the bacteriophage comprises a 5′ recognition site and a 3′ recognition site separated by 3 to 10 unspecified intervening nucleotides.

In some embodiments, the bacterial restriction modification system is the Type1 R-M system, accordingly, the 5′ and 3′ recognition sites of the bacteriophage are recognized by N- and C-terminal target recognition domains (TRD) of a specificity protein (also referred to herein as S-protein) of the bacteria. The “target recognition domains (TRD)” of a specificity protein refers to the domains with the specificity protein which recognize specific DNA sequences (the target sequence), guiding the restriction enzyme component of the Type1 R-M system to cleave the recognized DNA.

In some embodiments, the bacteriophage is a bacteriophage that infects at least one bacterium of the Bacteroidota phylum.

In more specific embodiments of the engineered bacteriophage, the Bacteroidota bacterium is of the genus Bacteroides.

In specific embodiments of the disclosed engineered bacteriophage, the Bacteroides bacterium is of the specie Bacteroides fragilis.

In embodiments of the disclosed engineered bacteriophage, the phenotype displayed by the Bacteroides fragilis that express type 1 R-M system S-proteins that recognize the recognition sites in the engineered bacteriophage, is the expression of outersurface molecules by the bacteria. In some embodiments, the phenotype may be also the expression of the specific type 1 R-M system S-proteins. In some embodiments, the bacterial outersurface molecules comprise at least one of PSA, PSF, PSB and/or CPS3.

In some embodiments, the bacteriophage disclosed herein is an engineered bacteriophage comprising at least one exogeneous recognition sequence of type 1 R-M system of B. fragilis specifically recognized by a specific S protein encoded by the HsdS gene of B. fragilis. The recognition sequence comprises a 5′ recognition site sequence of: GAC; and a 3′ recognition site selected from: GRTY, CTG, TCC and TGC; and/or the reverse complement thereof. The 5′ recognition site and a 3′ recognition site are separated by 3 to 10 intervening N nucleotides. It should be noted that A is adenine, G is guanin, C is cytosine, T is thymine, R is adenine (A) or guanin (G), Y is cytosine (C) or thymine (T), and N is any nucleic acid residue.

In yet some further embodiments, the bacteriophage of the present disclosure comprises at least one repeat of a recognition sequence comprising: (a), the sequence comprising GACNNNNNGATC, and/or the reverse complement thereof comprising the sequence GATCNNNNNGTC; and/or (b), the sequence comprising GACNNNNNCTG, and/or the reverse complement thereof comprising the sequence CAGNNNNNGTC. It should be understood that A is adenine, G is guanin, C is cytosine, T is thymine, R is A or G, Y is C or T, and Nis any nucleic acid residue.

Thus, in some embodiments, the engineered bacteriophage may comprise at least one repeat of the recognition sequence GACNNNNNGRTY (GACN₅GRTY) and/or the reverse complement thereof, specifically, RAYCNNNNNGTC (RAYCN₅GTC), wherein N can be any nucleotide (T, A, C, G), R, can be A or G, and Y can be C or T.

In some embodiments, the engineered bacteriophage may comprise at least one repeat of the recognition sequence GACNNNNNGATC (GACN₅GATC) and/or the reverse complement thereof, specifically, GATCNNNNNGTC (GATCN₅GTC), wherein N can be any nucleotide (T, A, C, G). Still further, in some embodiments, the engineered bacteriophage may comprise at least one repeat of the recognition sequence GACNNNNNNGATC (GACN₆GATC), and/or the reverse complement thereof.

In some embodiments, the engineered bacteriophage may comprise at least one repeat of the recognition sequence GACNNNNNNNGATC (GACN₇GATC), and/or the reverse complement thereof. In some embodiments, the engineered bacteriophage may comprise at least one repeat of the recognition sequence GACNNNNNNNNGATC (GACN₈GATC), and/or the reverse complement thereof. In some embodiments, the engineered bacteriophage may comprise at least one repeat of the recognition sequence GACNNNNNNNNNGATC (GACN₉GATC), and/or the reverse complement thereof. In some embodiments, the engineered bacteriophage may comprise at least one repeat of the recognition sequence GACNNNNNNNNNNGATC (GACN₁₀GATC), and/or the reverse complement thereof. In some embodiments, these recognition sequences are recognized by a B. fragilis S-protein having the 57/59 orientation. In some embodiments, such S-protein of the 57/59 orientation is encoded by the nucleic acid sequence as denoted by SEQ ID NO: 18.

In yet some further alternative embodiments, the engineered bacteriophage may comprise at least one repeat of the recognition sequence GACNNNNNCTG (GACN₅CTG), and/or at least one repeat of the reverse complement thereof, specifically, CAGNNNNNGTC (CAGN₅GTC), wherein N is any nucleotide (T, A, C, G). Still further, in some embodiments, the engineered bacteriophage may comprise at least one repeat of the recognition sequence GACNNNNNNCTG (GACN₆CTG), and/or the reverse complement thereof. In some embodiments, the engineered bacteriophage may comprise at least one repeat of the recognition sequence GACNNNNNNNCTG (GACN₇CTG), and/or the reverse complement thereof. In some embodiments, the engineered bacteriophage may comprise at least one repeat of the recognition sequence GACNNNNNNNNCTG (GACN₈CTG), and/or the reverse complement thereof. In some embodiments, the engineered bacteriophage may comprise at least one repeat of the recognition sequence GACNNNNNNNNNCTG (GACN₉CTG), and/or the reverse complement thereof. In some embodiments, the engineered bacteriophage may comprise at least one repeat of the recognition sequence GACNNNNNNNNNNCTG (GACN₁₀CTG), and/or the reverse complement thereof.

In some embodiments, these recognition sequences are recognized by a B. fragilis S-protein having the 57/58 orientation. In some embodiments, such S-protein of the 57/58 orientation is encoded by the nucleic acid sequence as denoted by SEQ ID NO: 16.

In some alternative embodiments, the engineered bacteriophage may comprise at least one repeat of the recognition sequence GACNNNNNNTCC (GACN₆TCC), and/or the reverse complement thereof.

In some embodiments, these recognition sequences are recognized by a B. fragilis S-protein having the 57/57 orientation. In some embodiments, such S-protein of the 57/57 orientation is encoded by the nucleic acid sequence as denoted by SEQ ID NO: 13.

In some embodiments, the engineered bacteriophage may comprise at least one repeat of the recognition sequence GACNNNNNNTGC (GACN₆TGC), and/or the reverse complement thereof. In some embodiments, these recognition sequences are recognized by a B. fragilis S-protein having the 57/60 orientation. In some embodiments, such S-protein of the 57/60 orientation is encoded by the nucleic acid sequence as denoted by SEQ ID NO: 20.

In yet some further embodiments, the engineered bacteriophage comprises an exogeneous nucleic acid sequence as denoted by SEQ ID NO: 10, comprising at least one of recognition sequence, specifically, the recognition sequence GACNNNNNGATC, and its reverse complement of GATCNNNNNGTC. In some embodiments, this recognition sequence is recognized by a specificity protein encoded by the bacterial (B. fragilis) genomic 57H/59T, regions, that comprise the nucleic acid sequence as denoted by SEQ ID NO: 18. In yet some further alternative or additional embodiments, the engineered bacteriophage used in the disclosed methods may comprises an exogeneous nucleic acid sequence as denoted by SEQ ID NO: 12, comprising at least one of recognition sequence, specifically, the recognition sequence GACNNNNNCTG, and its reverse complement of CAGNNNNNGTC. In some embodiments, this recognition sequence is recognized by a specificity protein encoded by the bacterial (B. fragilis) genomic 57H/58T, regions, that comprise the nucleic acid sequence as denoted by SEQ ID NO: 16.

A further aspect of the preset disclosure relates to a composition at least one engineered bacteriophage or any cocktail or mixture of the bacteriophages or any vehicle, matrix, nano- or micro-particle thereof. The engineered bacteriophage comprising at least one exogeneous recognition sequence of at least one bacterial restriction modification system (R-M), wherein said at least one recognition sequence is specifically recognized by at least one component of said restriction modification system of at least one bacterium displaying a desired phenotype. The recognition sequence comprises a 5′ recognition site and a 3′ recognition site separated by 3 to 10 unspecified intervening nucleotides. In some embodiments, the composition further comprises at least one of pharmaceutically acceptable carrier/s, diluent/s, excipient/s and additive/s. In some embodiments, the compositions comprise any of the bacteriophages disclosed by the present disclosure in connection with all aspects of the present disclosure, in any effective amount thereof (e.g., provide 0.1×10⁸ to 1×10¹², plaque forming units (PFU) of the disclosed bacteriophage, per Kg of body weight of the administered subject), as disclosed in the present disclosure. In general terms, under compositions herein is meant predominantly pharmaceutical compositions, meaning that such compositions would comprise a therapeutically effective amount of at least one active agent, i.e. a modulator according to the invention, and optionally, at least one pharmaceutically acceptable carrier. The term ‘pharmaceutically acceptable’ means approved by a regulatory agency or listed in the U.S. Pharmacopeia or other generally recognized pharmacopeia for use in animals, and more particularly in humans. The term ‘carrier’ denotes to a diluent, adjuvant, excipient, or vehicle with which the therapeutic is administered. Example of such pharmaceutical carriers are sterile liquids, such as water and oils, including those of petroleum, animal, vegetable or synthetic origin, such as peanut oil, soybean oil, mineral oil, sesame oil and the like. Suitable pharmaceutical excipients may include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A composition can further contain minor amounts of wetting or emulsifying agents, or pH buffering agents. The compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. In specific embodiments, the compositions of the invention may be formulated in accordance with routine procedures as pharmaceutical compositions adapted for intravenous administration in humans. Where necessary, the composition may also include a solubilizing agent and a local anesthetic such as lidocaine to ease pain at the site of the injection. Where the composition is administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical compositions of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration. Local administration to the area in need of treatment may be achieved by, for example, by local infusion during surgery, topical application, direct injection into the specific organ, etc. More specifically, the compositions used in the methods and compositions of the invention, described herein after, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). It should be noted that any of the administration modes discussed herein, may be applicable for any of the methods of the invention as described in further aspects of the invention herein after. Compositions and formulations for oral administration may include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable. Pharmaceutical formulations adapted for rectal administration may be presented as suppositories or enemas. Pharmaceutical formulations adapted for vaginal administration may be presented as pessaries, tampons, creams, gels, pastes, foams or spray formulations. Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question, for example those suitable for oral administration may include flavoring agents. The compositions of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis. Formulations for ocular and aural administration may be formulated to be immediate and/or modified release. Modified release includes delayed, sustained, pulsed, controlled, targeted, and programmed release. In specific embodiments, the unit dosage formulations are those containing a daily dose or sub-dose, as herein above recited, or an appropriate fraction thereof, of an active ingredient. Of particular relevance are formulations of compositions of the invention adapted for use as a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as liposomes or polyelectrolyte multilayer capsules, incorporate nanoparticle (NP) actuators.

A further aspect of the present disclosure relates to a method for modulating the phenotype of a bacterial population. The method comprising the step of contacting the bacterial population with an effective amount of at least one engineered bacteriophage comprising at least one exogeneous recognition sequence of at least one bacterial restriction modification system (R-M). It should be noted that the at least one recognition sequence is specifically recognized by at least one component of the restriction modification system of at least one bacterium displaying a desired phenotype. The recognition sequence comprises a 5′ recognition site and a 3′ recognition site separated by 3 to 10 unspecified intervening nucleotides, thereby enriching bacteria displaying the desired phenotype in the bacterial population.

In some embodiments, the bacteriophage modulates phase variation of at least one locus (e.g., a gene locus) of at least one bacterium in the bacterial population.

In yet some further embodiments, the engineered bacteriophage used in the disclosed methods is as defined by the present disclosure.

In some embodiments, the bacterial population is in a subject suffering from at least one immune-related disorder.

In yet some further embodiments, the subject is suffering of an immune-related disorder, specifically, at least one of a proliferative disorder, an inflammatory disorder, an autoimmune disorder, an immune-deficiency condition and/or an infectious disease.

The present disclosure provides modulatory methods, therapeutic methods bacteriophages and compositions that are directed to modulate hosts, specifically, subjects in need thereof. A subject in accordance with the present disclosure may be at least one organism of the biological kingdom Animalia or of the biological kingdom Plantae. Still further, the present disclosure relates to the treatment of subjects or patients in need thereof. By “patient” or “subject in need”, in accordance with all aspects of the preset disclosure, it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the present disclosure are intended for mammals. By “mammalian subject” means any mammal for which the proposed therapy is desired, including human, livestock, equine, canine, and feline subjects, most specifically humans.

In some embodiments, the methods of the present disclosure may be applicable for any organism of the biological kingdom Animalia. In more specific embodiments, such organism may be any unicellular or multicellular invertebrate or vertebrate organism. More specifically, invertebrates, may be organisms of the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida—Segmented worms like earthworm and the Phylum Echinodermata—Echinoderms. Still further, in some embodiments, the methods of the present disclosure may be applicable for any vertebrate organism, specifically, any organism derived from any of the vertebrates groups that include Fish, Amphibians, Reptiles, Birds and Mammals (e.g., Marsupials, Primates, Rodents and Cetaceans). In some particular embodiments, the methods of the present disclosure may be applicable for a mammal (specifically, at least one of a human, Cattle, rodent, domestic pig (swine, hog), sheep, horse, goat, alpaca, lama and Camels). More specifically, in some embodiments, as indicated herein, the methods of the present disclosure may be applicable for a vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans).

Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish.

Still further, in some embodiments, the subject of the of the preset disclosure may be any one of a human or non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the subject of the present disclosure may be a mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the present disclosure may be applicable for any organism of the order primates. More specifically, primates are divided into two distinct suborders, the first is the strepsirrhines that includes lemurs, galagos, and lorisids. The second is haplorhines—that includes tarsier, monkey, and ape clades, the last of these including humans. In yet some further embodiments, the present disclosure may be applicable for any organism of the subfamily Homininae, that includes the hylobatidae (gibbons) and the hominidae that includes ponqunae (orangutans) and homininae [gorillini (gorilla) and hominini ((panina (chimpanzees) and hominina (humans))]. Thus, in some embodiments, a subject as disclosed herein relates to a human subject. In some embodiments, the human subject may be of any sex, ethnic group, age or physical or mental condition.

In some specific embodiment, the methods of the present disclosure may be applicable for a mammal that may be at least one of a Cattle, domestic pig (swine, hog), sheep, horse, goat, alpaca, lama and Camels.

More specifically, the subject the present disclosure as well as the methods disclosed herein above offer great economic advantage for any industrial or agricultural use of animals, specifically, livestock. Thus, in some specific embodiments, the present disclosure may be applicable for mammalian livestock, specifically those used for meat, milk and leather industries. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the subject of the present disclosure may be Cattle, colloquially cows, that are the most common type of large, domesticated ungulates, that belong to the Bovidae family.

In yet some further embodiments, the organism applicable in the methods of the present disclosure, may be avian organisms. In yet some further specific embodiments, the present disclosure may be suitable for birds. More specifically, domesticated and undomesticated birds are also suitable organisms for the present disclosure.

Therefore, in certain embodiments, the avian organism of the preset disclosure may be any one of a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. The term “avian” relates to any species derived from birds characterized by feathers, toothless beaked jaws, the laying of hard-shelled eggs, a high metabolic rate, a four-chambered heart, and a lightweight but strong skeleton. The term “hen” includes all females of the avian species.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to +10%. The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Experimental Procedures

### Mice

All mouse work was in accordance with protocols approved by the local IACUC committee under approval numbers: IL-151-10-21 and IL-1May 6, 2021.

Male, 4-5 weeks-old Germ-Free (GF) C57BL/6 mice from the Technion colony were used. Mice were housed and maintained in a GF care facility and were provided with food and water ad libitum; they were exposed to a 12:12 h light-dark cycle at room temperature.

GF mice were administered twice (on day 0 and on day 2), by gavage, with B. fragilis NCTC 9343 strain and bacteriophage Barc2635, B. fragilis only NCTC 9343 strain or growth media as control. On day 10 mice were sacrificed for bacteria and immune phenotyping.

qPCR and Primers

DNA was extracted from fecal samples using ZymoBIOMICS DNA Miniprep Kit [Zymo research]. The ‘ON/OFF’ orientation of the PSA gene in the extracted DNA samples was determined by quantitative polymerase chain reaction (qPCR) using SYBR® Green mix [Thermo Fisher Scientific]. Two sets of primers were designed to target the PSA locus. One set was used as a proxy to the number of bacteria in the samples and targeted UpaY, the first gene immediately downstream to the promoter region. The second set of primers targeted the promoter region and would only produce a product when the orientation is ‘ON’. The ratio of ‘ON/OFF’ PSA orientation in the samples was calculated using the 2-ΔΔCT method calculated against the PSA locked ‘ON’ results (100% ‘ON’ orientation).

UpaY mRNA levels were determined by RT-qPCR. RNA was extracted from fecal samples using zymoBIOMICS RNA miniprep kit [Zymo research]. Reverse transcription of RNA to cDNA was performed using the qScript cDNA Synthesis Kit [Quantabio]. UpaY mRNA levels were determined by qPCR using SYBR® Green mix [Thermo Fisher Scientific] with primers against rpsL as a reference gene. The 2-ΔΔCT method was employed for the specificity fold change tests.

TABLE 1
qPCR primers

| | | SEQ |
|---|---|---|
| Seq name | Sequence | ID NO. |
| B_Frag_upaY_F | CGCTCGGACAAAGAAGGACC | 1 |
| B_Frag_upaY_R | ACTTCTACCCTACGACGACGA | 2 |
| B_Frag_PSA_R | GGTGTTCCAAAAGACGAACGT | 3 |
| B_Frag_PSA_F | TGTGTAAATGATAGGAGGCTAGGG | 4 |
| rpsL_F | CCGAACTCTGCAATGCGTAA | 5 |
| rpsL_R | CGCGAACCAGTACGATTGAG | 6 |

### Sequencing

DNA samples underwent quality control by Qubit fluorescence analysis to determine concentration of DNA for downstream analysis (ThermoFisher, Cat. Q32850). Libraries were prepared using the Illumina Tagmentation DNA prep streamlined library preparation protocol according to manufacturer's instructions with a minimum of 50 ng of DNA starting mass and 8 cycles of PCR enrichment, ending with a fragment size of 550 bp. IDT for Illumina DNA/RNA UD indexes and Nextera DNA CD indexes were used (Illumina IDT, Cat. 20027213; Illumina Nextera, Cat. 20018708).

All libraries were diluted to 15 μM in 96-plex pools and validated on 100-cycle paired-ends read Miseq V2 runs (Illumina, Cat. MS-102-2002), before shipping to the US at 4 nM for sequencing on the Novaseq 6000 in S4 mode at 96-plex in a 300-cycle paired-end reads run, with an estimated read depth of 30 Gbp per sample (Illumina, Cat. 20028312). Final loading concentration of 600 pM. All sequencing runs were performed with a spike-in of 1% PhiX control library V3 (Illumina, Cat. FC-110-3001).

### Taxonomic Profiling

Community profiling was performed using metaphlan [Goldberg A, et al. Genome Med. 2014; 6 (11): 112] v4.0.059 with mpa database vJan21. For each sample, the forward reads were first aligned against the mpa database using bowtie2 v2.3.5.160 (flags—sam—no—hd—sam—no—sq—no—unal—very-sensitive). Next, the resulting sam file was analyzed by metaphlan [Goldberg A, et al. Genome Med. 2014; 6 (11): 112] with default parameters. In each analysis, species at abundance 489 <=0.1% were ignored.

### Microbiome Analysis

Statistical analysis of Sequenced data was initially performed using MicrobiomeAnalyst [Dhariwal et al., Nucleic Acids Res. 2017] followed by comprehensive analysis using R packages: Phyloseq [McMurdie & Holmes, PLOS One 2013], Vegan [Simpson, 2009] and DESeq2 [Love et al., Genome Biol. 2014]. Differences in microbial taxa and functional modules were assessed by differential abundance analyses using DESeq2 [Love et al., Genome Biol. 2014].

### Identification of Phase Variable Sites

Phasefinder [Jiang X, et al. Science 363 (6423): 181-187. 2019] (v1.0) was used to identify phase variable sites in metagenomics samples. The default parameters of Phasefinder were used. The results were filtered by removing identified sites with <20 reads supporting either the forward or reverse orientations combined from the paired-end method, and mean Pe_ratio <1% across all samples and sites within coding regions of rRNA products.

### Bacteriophage Isolation

Isolation of Bacteriophage from Sewage

Bacteroides fragilis NCTC 9343 was used as the host strain. Bacteroides Phage Recovery Medium (BRPM) was used for the growth of the host bacterium as well as for plaque assays [Tartera C, Araujo R, et al. Appl Environ Microbiol. 1992; 58 (8): 2670; ISO 10705-4:2001, Water Quality—Detection and Enumeration of Bacteriophages—Part 4: Enumeration of Bacteriophages Infecting Bacteroides Fragilis: ISO/TC 147/SC 4: Amazon.Com: Books. International Organisation for Standardisation; 2001]. Manipulations were done onto the bench and cultures were incubated under anaerobic conditions using GasPak jars (Becton, Dickinson and Cia. BBL, Franklin Lakes, NJ).

For isolation of bacteriophages, inflowing raw sewage for a waste-water treatment plant (WWTP) from Barcelona (Spain) was filtered through low protein binding 0.22 μm pore size polyethersulfone (PES) membrane filters (Millex-GP, Millipore, Bedford, Massachusetts) to remove bacteria. Isolated plaques were obtained by the double-agar layer technique [Adams M H. Bacteriophages. Bacteriophages. Published online 1959]. Briefly, tubes containing 2.5 ml of soft BPRM-agar kept at 45° C. were inoculated with 1 ml of an exponential growth phase culture (OD600=0.3, corresponding to ca 2×10⁸ CFU/ml) of the host bacteria grown in BPRM broth and 1 ml of the filtered sewage sample. After gently mixing, the contents of each tube were poured onto a plate of BRPM-agar and incubated inside GasPak (BBL) jars at 37° C. Plaques were clearly spotted after 18 h of incubation.

For phage isolation, discrete well-isolated plaques were stabbed with a sterile needle and inoculated in a tube containing 5 ml of BRPM broth. Then 1-ml of a culture of B. fragilis NCTC 9343 in exponential grown was inoculated into the tube, which was then incubated for 18 h at 37° C. After incubation, an aliquot of the culture was treated with chloroform (1:10 (v; v), vigorously mixed for 5 minutes and centrifuged at 16.000×g for 5 minutes [García-Aljaro C, et al. Methods Mol Biol. 2018; 1693:11-22]. The supernatant containing the phage suspensions were further filtered through low protein binding 0.22 μm pore size polyethersulfone (PES) membrane filters (Millex-GP, Millipore, Bedford, Massachusetts), diluted and plated as indicated in the previous paragraph to verify the uniformity of the plaques. Then, one well differentiated plaque was stabbed and the whole operation repeated to obtain a high titer, over 10⁹ plaque forming units (PFU), phage suspensions.

Isolation of Bacteriophages from Feces of IBD Patients and Administration to Mice Monocolonized with B. fragilis Fecal samples from IBD patients were suspended in sterile PBS in a ratio of 100 mg: 1 ml. The samples were centrifuged at 4,500×g for 15 minutes, and supernatants were collected and filtered using the Medical Millex-VV Syringe Filter Unit, 0.22 μm, PVDF membrane. To check whether these filtrates contain phages specific for Bacteroides fragilis NCTC 9394, B. fragilis was grown in Bacteroides Phage Recovery Medium (BPRM), and the following day the culture was diluted 1:10. At OD-0.5, 300 ul of B. fragilis was infected with 30 ul of the fecal supernatant, incubated for 5 minutes in 37° C., and the culture (containing the bacteria and the supernatant) was mixed with Top Agar and plated in BPRM plates. The following day plaques were formed for some samples. To purify these plaques, 50 ul of sterile Sodium chloride, Magnesium sulphate (SM) buffer was poured to the center of the plaque and the plaque was picked and stored in 1 ml SM phage storage buffer. To propagate these phages, 1 ml of B. fragilis grown in BPRM was infected with each plaque alone until clear lysis, and then the lysed culture was filtered using the Medical Millex-VV Syringe Filter Unit, 0.22 μm, PVDF membrane. To quantify the stocks of phages, B. fragilis was grown in BPRM to log phase (OD=0.5), 300 ul of bacteria was infected with 30 ul of several serial dilutions of filtered phage. The cultures were incubated for 5 minutes and plated together with Top Agar on BPRM plates. The following days plaques were counted, and the stock was quantified. To evaluate in vivo the effect of each of the isolated bacteriophages on the PSA orientation of B. fragilis, germ-free mice were first gavaged with 200 ul B. fragilis NCTC 9394 (10~8 CFU), after two days, feces were collected (TO), and 100 ul of 10∧9 of each phage were gavaged separately. After 10 days, mice were sacrificed, and feces (T10) were collected. DNA was eluted using ZymoBIOMICS DNA Miniprep Kit, and qPCR was used to check for PSA orientation.

### Phage Dynamics

Adsorption assays were done by infecting B. fragilis NCTC 9343 cells grown at 37° C. anaerobically to mid-exponential phase (OD₆₀₀=0.4−0.5) in BPRM media with BARC2635 at a multiplicity of infection (MOI) of >1. Quantification of infective phages at different timepoints was determined by filtering 100 ul of the sample through a 0.22 μm filter to a new tube, and serial dilutions of the filtrate were used for plaque assays.

One-step growth-curve experiments were conducted to assess latent period and burst size. Exponentially growing B. fragilis NCTC 9343 cells were grown at 37° C. anaerobically to mid-exponential phase (OD₆₀₀=0.4−0.5) in BPRM media. The bacteria were infected with BARC2635 at a multiplicity of infection (MOI) of 0.01, and then incubated for 5 minutes. The bacteria with phages were centrifuged at 6000×g for 10 min at 4° C. The cell pellet was resuspended in 3 ml BPRM media. Quantification of infective phages at different timepoints was determined by filtering 100 ul of the sample through a 0.22 μm filter to a new tube, and serial dilutions of the filtrate were used for plaque assays. Burst size was estimated from the number of free infective phages at the end of the lytic cycle minus the number at maximal adsorption (35~ min), divided by the number of infected cells.

Plaque assays were done by the double-agar layer technique [Adams M H. Bacteriophages. Bacteriophages. Published online 1959]. Briefly, tubes containing 4 ml of soft BPRM-agar kept at 45° C. were inoculated with 0.2 ml of an exponential growth phase culture (OD₆₀₀=0.4, corresponding to ca 3×10⁸ CFU/ml) of the host bacteria grown in BPRM broth and 10 μl of the diluted filtered samples. After gently mixing, the contents of each tube were poured onto a plate of BRPM-agar and incubated anaerobically at 37° C. Plaques were clearly spotted after 18 h of incubation.

### Phage Genome Sequencing

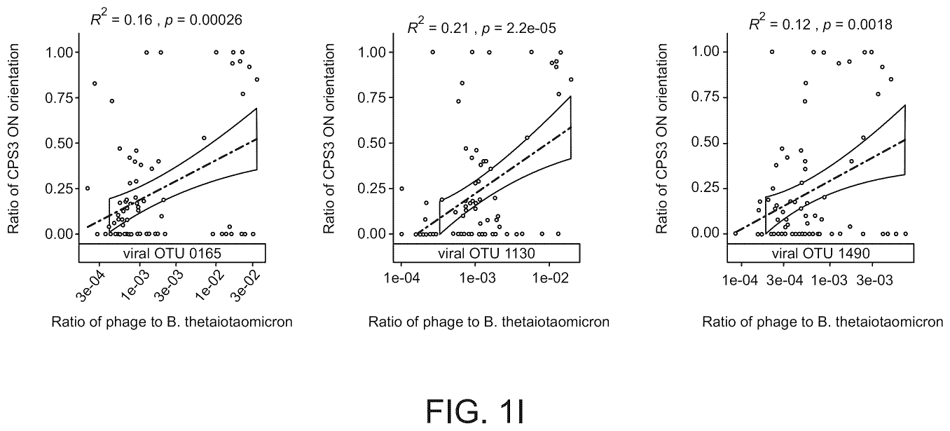

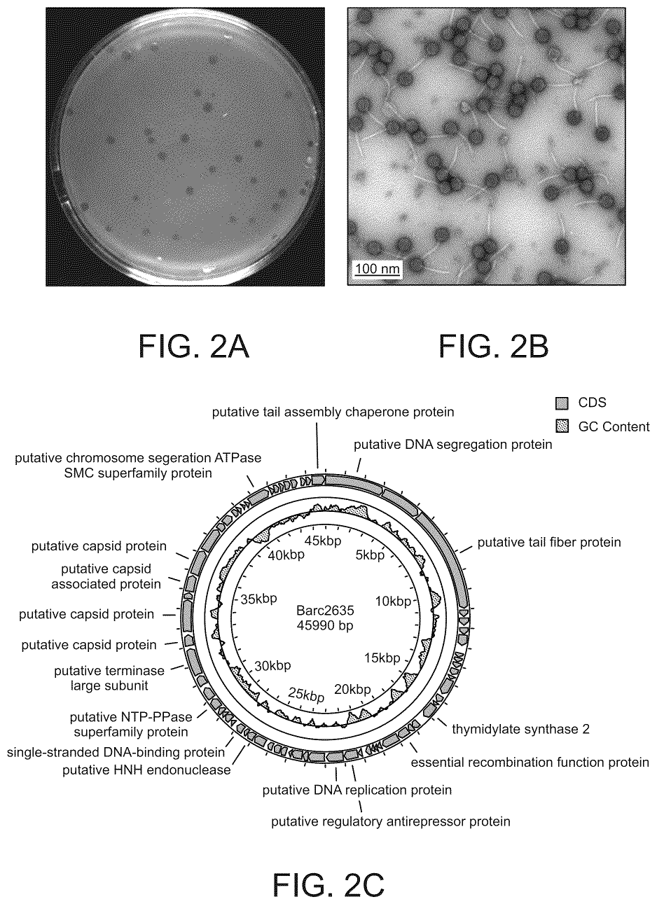

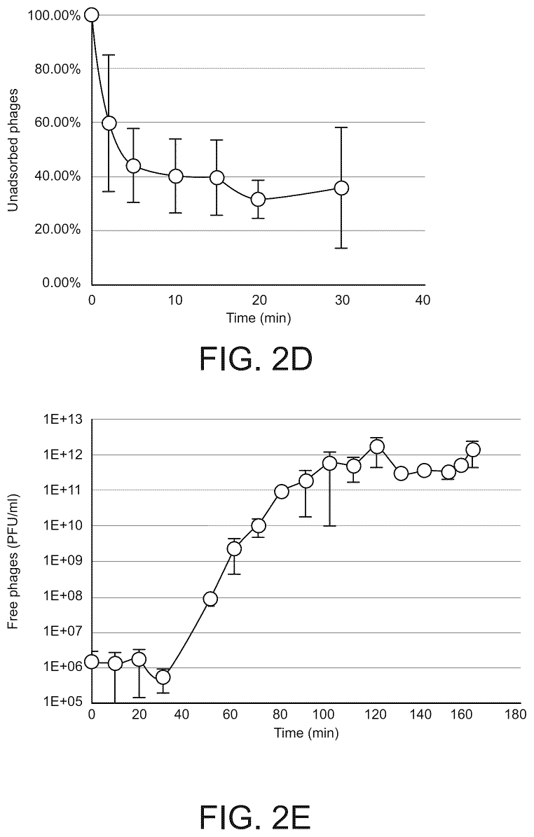

5 hrs induction of phage DNA, phage particles were PEG-precipitated (6,000-12,000 MW, 8%), and isolated by a CsCl gradient; 33 g, 41 g, 55 g in 50 ml TM buffer (50 mM Tris-Cl pH8.0, 10 mM MgCl2), ultracentrifuged at 40,000 rpm for 1.5 hrs, and dialyzed overnight in 100 mM Tris ph7.5, 1M NaCl, 1 mM EDTA 56. Genomic DNA was extracted using phenol-chloroform as described [Tropini C, et al. Cell. 2018; 173 (7): 1742]. Illumina sequencing of Bacteroides phage Barc2635 was performed at the Biopolymers Facility, Harvard Medical School, Department of Genetics, producing paired-end reads of 150 bp. Adapter sequence removal and quality trimming was performed using BBDuk, part of the BBTools (v 37.50) suite of programs. The reads were further screened against NCBI's UniVec_Core database (build 10.0) and the B. fragilis NCTC 9343 genome sequence using blastn and reads that returned a significant hit to either were removed. The phage genome was assembled de novo using Velvet 1.2.10 under a k-value determined by Velvet Optimizer (v. 2.2.5). The genome was annotated using an in-house customized version of Prokka v1.12, submitted to NCBI, and assigned GenBank accession MN078104. Phage Genome map FIG. 2C was visualized using the online tool Proksee (https://proksee.ca/, accessed on 21 Jan. 2023).

### Viral OTUs Multiple Alignment

Alignments and phylogenetic tree of whole genomes of viral OTUs identified as bacteriophages against B. fragilis33 as well as bacteriophage Barc2635 were generated using MAFFT online service (version 7). Multiple sequence alignment was done using the default settings of the site. Phylogenetic tree was constructed using the average-linkage method with 1000 bootstrap replicates and was visualized with R package ggtree [Yu G. et al. Curr Protoc Bioinformatics. 2020; 69 (1): e96]. (version 3.4.4).

### MinION Library Preparation and Sequencing

The specificity region of BF9343_1757-1760 was amplified by PCR from a population of bacteria grown in vitro and in vivo from fecal content as described above. The primers annealed outside the invertible region. The amplicons were purified by Wizard SV Gel and PCR Clean-Up System (Promega) and measured by nanodrop.

Primers: Type1RM_hsdS_F: GACAATCGAGATGAAGAACAAC, as denoted by SEQ ID NO: 7, and Type 1RM_hsdS_R: CCATAGGCGTATGATTTCCTG, as denoted by SEQ ID NO: 8. DNA quantity was measured again using Qubit fluorometry (Thermo Fisher Scientific, Waltham, MA, USA). Nanopore sequencing libraries were prepared from 200 fmol purified amplicons using Ligation Sequencing Kit 1D (SQK-LSK109) and PCR-free Native Barcoding Expansion Kit (EXP-NBD104) (Oxford Nanopore Technologies, Oxford, England). The barcoded libraries were loaded and sequenced on the MinION device controlled by MinKNOW software (v.19.12.5) using MinION flow cells (FLO-MIN106D R9.4.1, Oxford Nanopore Technologies, Oxford, England) after quality control runs. The raw data were base called and demultiplexed by Guppy Basecalling Software (v. 3.3.3+fa743a6).

### MinION Data Analysis

Adapters and barcodes sequences were removed from the reads using Porechop (v0.2.4, available from https://github.com/rrwick/Porechop). Reads were oriented using the ‘Preparing Reads for Stranded Mapping’ protocol (Eccles, D.A. (2019). Protocols.io, Vol. 2019, pp. Protocol) [Eccles DA. Preparing Reads for Stranded Mapping. Published 2022. Accessed Feb. 11, 2023. https://www.protocols.io/view/preparing-reads-for-stranded-mapping-5qpvon2zzl40/v7]. The reads were aligned to the PCR forward primer using LASTAL (v.1060) [Frith MC. et al. BMC 898 Bioinformatics. 2010; 11 (1): 1-14], and then reverse-complemented the reverse-oriented reads. The reads were combined to an all forward oriented file and cropped to the first 1300 bases using Trimmomatic (v.0.39) [Bolger A M, et al. Bioinformatics. 2014; 30 (15): 2114-2120]. The reads were then split according to their alignment to the 1757-57 or 1757-60 5″ half sequences using LASTAL. Reads were mapped to the full sequences with Minimap2 (v.2.17-r941) using the-for-only and asm20 options. Mapped read counts were extracted from the Minimap2 SAM output using SAMtools (v.1.7) [Li H, et al. Bioinformatics. 2009; 25 (16): 2078-2079].

MinION sequence data has been deposited in the NCBI sequence read archive (SRA) under the BioProject accession number: PRJNA948162. These sequences are the type I RM genome region of B. fragilis. Sequences were used to evaluate percentage of each phase orientation within the type 1 RM genome region. Accession number will be provided.

Fecal filtrates of patients Recruitment of IBD patients for this study was conducted at the Rambam Health Care Campus (RHCC). The study was approved by the local institutional review boards with study numbers 0052-17 and 0075-09 in which all patients consented to be included in it. During therapy, patients were treated either with Infliximab or Humira for at least two weeks. Fecal samples were collected by patients at home prior to their clinic visit and collected from each at the hospital. The samples were then stored at −80° C. until they were shipped to the laboratory for analysis.

Calprotectin concentration in stool was determined as a marker of intestinal inflammation. Fecal supernatants were prepared after suspension in 1:10 sterile PBS and centrifugation for 15 minutes in 4500×g. Enzyme-linked immunosorbent assay (ELISA) to measure calprotectin concentration was performed using Mouse S100A8/S100A9 Heterodimer kit according to the manufacturer protocol [R&D systems] or human KIT LIAISON calprotectin (catalogue No. 318960) according to the manufacturer's instructions. The levels of calprotectin in the fecal samples were used as a measure of disease activity in IBD patients.

In Vitro Assays with Fecal Filtrates from IBD Patients

Bacteroides fragilis NCTC 9343 was grown in Brain Heart Infusion medium with supplements (BHIS) to OD 600~0.6 and centrifuged for 5 minutes in 4500×g. Then, bacterial pellets were washed twice with sterile PBS to discard of remaining BHIS components and then suspended with 1 mL M9 minimal media.

Patient fecal samples were suspended in 1:5 sterile PBS, centrifuged for 15 minutes in 4500×g. Supernatants were collected and filtered using the Medical Millex-VV Syringe Filter Unit, 0.22 μm, PVDF membrane. For the in vitro assay, B. fragilis was cultured in patient's fecal filtered supernatants and M9 minimal media in 1:25:25 ratio, respectively. Subsequently, 200 ul of each culture was collected for DNA extraction at OD600~0.6.

### Gut Lamina Propria Preparation

For lamina propria immunophenotyping, mice colons were removed by cutting the colon from the cecum-colon junction to the anus. Fat tissue was carefully removed from colon tissue and further proceeded for single cell suspension preparation using lamina propria dissociation kit (Miltenyi), according to the manufacturer's protocol.

### Flow Cytometry

Cell preparations for flow cytometry analysis were done in 5 ml tubes or U shape 96 wells plates. Single cells were washed with PBS and stained for live/dead staining using 1:1000 in PBS, Zombie fixable viability dye (Biolegend) for 10 minutes, at room temperature, and washed once with FACS buffer, by centrifuge at 300 ×g for 5 minutes. For Fc receptor (FcR) blocking, cells were incubated with 0.5 μg CD16/CD32 antibody for 10 minutes on ice and proceeded to further staining without a washing step. Extracellular markers were stained with the relevant antibody panels for 30 minutes on ice and washed twice with FACS buffer, by centrifuge at 300 ×g for 5 minutes. After the last wash, cells were fixed with Foxp3 Fixation/Permeabilization working solution (Thermo) for 16 hours at 4° C. in the dark. For Intracellular staining, cells were permeabilized using 1X Foxp3 permeabilization buffer (Thermo) according to the manufacturer protocol. For intracellular blocking, 2 μl of 2% rat serum (Stemcell technologies) was added to each well for 15 minutes at room temperature and proceeded to further staining without a washing step.

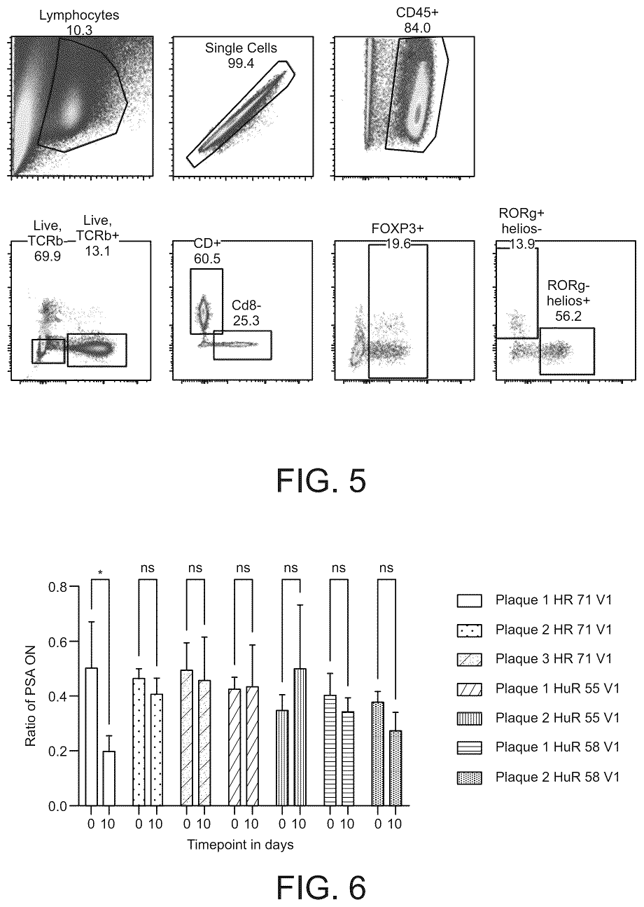

For bacterial polysaccharides expression analysis, bacteria were isolated from feces of monocolonized mice with B. fragilis and with or without bacteriophage. Feces were suspended in 1:10 ice cold PBS (mg/ul) and centrifuged at 300×g for 5 minutes, 4° C. Supernatants were separated from pellets and further centrifuged at 4500×g for 5 minutes, 4° C. Bacterial pellets were resuspended in an ice cold FACS buffer, 1:10 from initial PBS suspension. 100 μl of resuspended bacteria were incubated with 1:1000 Rabbit anti B. fragilis PSA for 30 minutes at 4° C. Bacteria were washed twice using an ice cold FACS buffer by centrifuge at 4500 ×g for 5 minutes and then incubated with a donkey anti rabbit fluorophore conjugated secondary antibody. After staining steps, the bacteria were washed twice with an ice cold FACS buffer and finally resuspended in 500 ul ice cold PBS plus 1:1000 Hoechst dye and analyzed by flow cytometry using FSC and SSC thresholds of 1000, and logarithmic scale. Gating strategy is detailed in FIG. 5.

### Example 1

Investigating viral association to genomic orientation of the Polysaccharide A promoter of B. fragilis

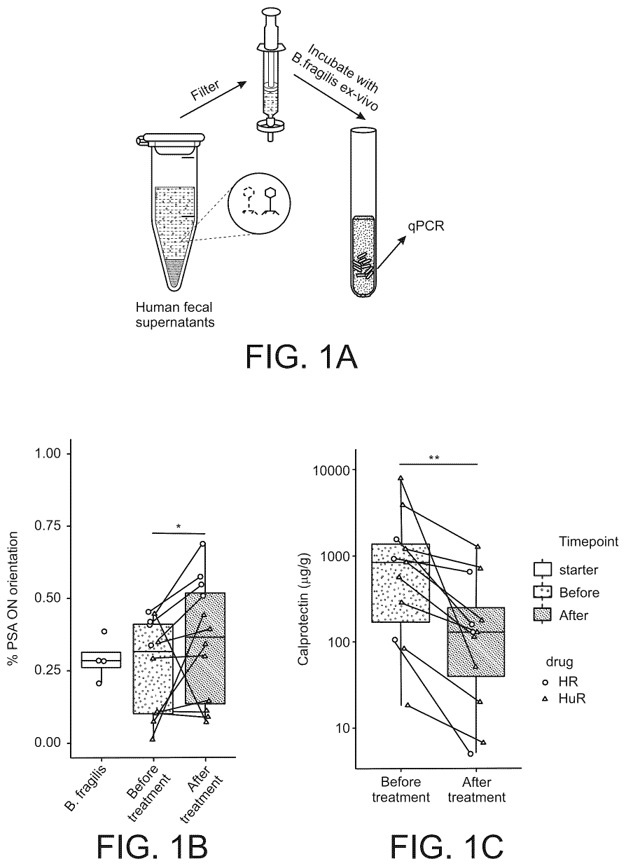

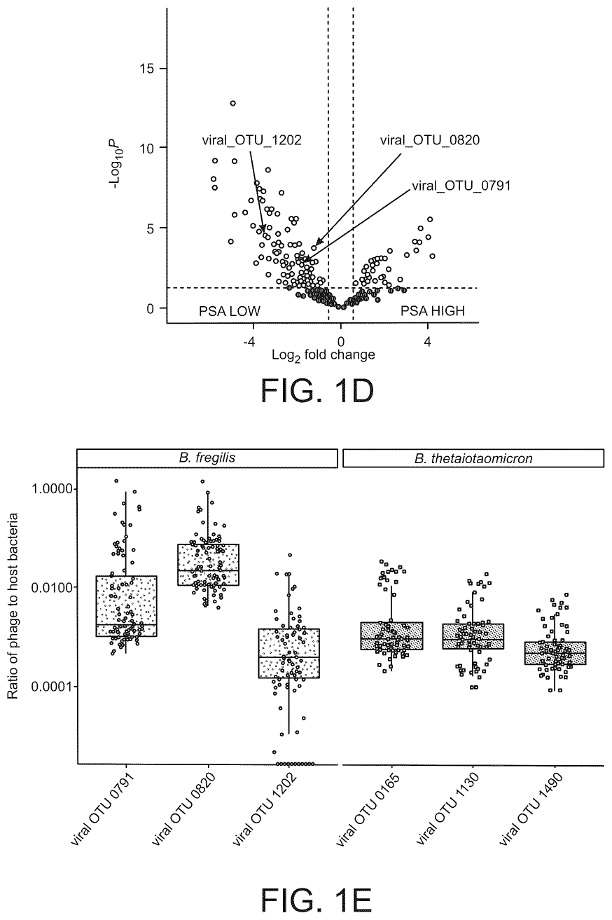

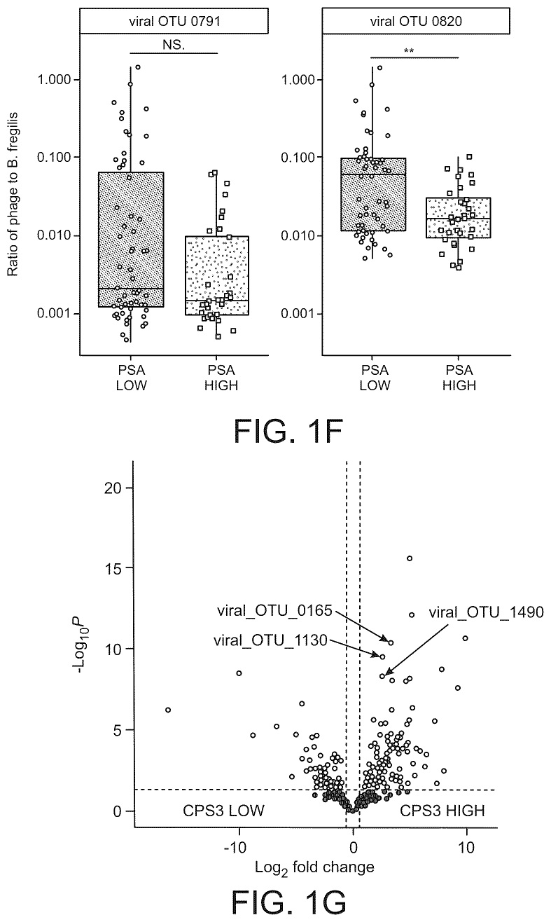

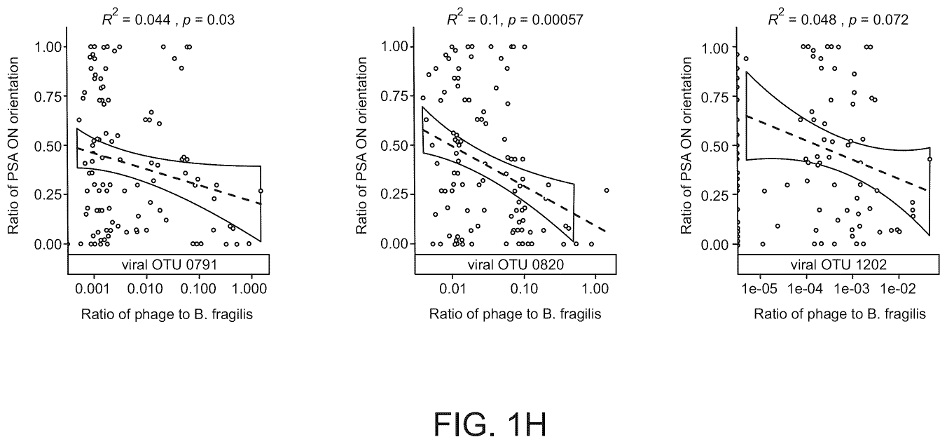

The inventors recently identified multiple phase variable genomic regions correlated with disease states including the anti-inflammatory polysaccharide A of B. fragilis, which is turned ‘OFF’ under inflammation. The inventors sought to examine whether the gut inflamed environment can induce bacterial genomic phase variations of polysaccharide A promoter of B. fragilis. To do so, B. fragilis was exposed to fecal filtrates from IBD patients. CD and UC patients were recruited from the Rambam Health Care Campus (RHCC). Fecal samples were collected before and after infliximab (IFX) or Humira (HuR) therapy, both are antibodies targeted against tumor necrosis factor-α (TNF-α), an inflammatory cytokine, which is increased in IBD patients. B. fragilis NCTC9343 was cultured in fecal filtrates until reaching mid log phase, and subsequently DNA was extracted for qPCR analysis of the PSA promoter orientation, as illustrated in the experimental scheme of FIG. 1A. This analysis revealed that B. fragilis exposed to fecal filtrates of patients before treatment, during inflammation, showed higher ratios of the population with PSA promoter ‘OFF’ orientation, whilst B. fragilis exposed to fecal filtrates after treatment showed higher ratios of the PSA promoter ‘ON’ orientation, (FIG. 1B). This observation was in line with the fecal Calprotectin concentrations, which were reverse correlated with the PSA promoter orientation (FIG. 1C). These results demonstrated that the population of bacteria with the PSA promoter in each orientation vary in the inflamed and non-inflamed gut, with a higher percentage of the population in the ‘OFF’ orientation under inflammatory conditions, and a shift towards the ‘ON’ orientation following reduction in inflammation (FIG. 1B, 1C). This change could be due to alterations of the inversion under these conditions or due to selection of a preferred promoter state. The fecal filtrates contain a mixture of bacterial and host metabolites, cytokines, antibodies, viruses, bacteriophages, and more. Bacteriophages have been observed to induce phase variation in bacterial cells, leading to the switching ‘OFF’ of specific polysaccharides loci or increased expression of alternative polysaccharides with possible protective effects [9] . . . . Moreover, they have been shown to be associated with intestinal inflammation and IBD in several studies [10, 11], including Nishiyama H, et al (2020) who characterized temperate bacteriophages and their bacterial hosts in the IBDMDB metagenomics database. Here, the inventors used the phage sequences from IBDMDB database, and compared the relative abundances of these bacteriophages between samples that displayed lower (<40%) or higher (>60%) ratios of the PSA promoter ‘ON’ orientation. Differential abundance analysis revealed that more bacteriophages were correlated with the ‘OFF’ orientation of PSA (FIG. 1D), three of which were predicted to infect B. fragilis by Nishiyama H, et al (2020).

To assess whether the bacteriophages' relative abundances were accompanied with lower relative abundance of B. fragilis, the relative abundance of phage to host ratios were compared between lower and higher ‘ON’ ratios of the PSA promoter orientation. The two viral OTUs with the highest phage to host ratios in patients, OTUs 0791 and 0820, were also correlated with low ratios of the PSA promoter ‘ON’ orientation (i.e. with the PSA promoter ‘OFF’ orientation) (FIG. 1E, 1F and 1H). Intriguingly, viral OTUs 0791 and 0820 were found to be more abundant in active CD and UC [Nishiyama H, et al. 2020].

The same analysis was conducted comparing samples with high to low ratios of B. thetaiotaomicron's CPS3 promoter ‘ON’ orientations. Although there were differences in the virome compositions between the groups, no B. thetaiotaomicron associated bacteriophages were correlated with the ‘OFF’ orientation of the CPS3 promoter (i.e., the orientation which the inventors identified as associated with the disease) (FIG. 1G). The inventors found 3 bacteriophage OTUs, which were previously predicted to infect B. thetaiotaomicron [Nishiyama H, et al. Microorganisms. 2020; 8 (11): 1-15], OTUs 0165, 1130, and 1490, slightly associated with the ‘ON’ orientation of the CPS3 promoter (the orientation which found to be associated with healthy controls), (FIGS. 1G and 1I). In addition, these bacteriophages displayed low phage to host ratios (FIG. 1E). Additional bacteriophages found to be associated with bacterial population presenting the ‘ON’ orientation of CPS3 include the viral OTU 0115, predicted to infect: M. 0027 Bacteroides, M. 3018 Bacteroides faecis; the viral OTU 0873, predicted to infect: M. 0099 Bacteroides ovatus, M. 0181 Bacteroides xylanisolvens; the viral OTU 1100, predicted to infect: M. 0619 Bacteroides vulgatus, M. 1162 Bacteroides ovatus; and the viral OTU 1014, predicted to infect: M. 0010 Bacteroides uniformis, M. 0025 Bacteroides stercoris, M. 0185 Bacteroides stercoris, M. 3027 Bacteroides massiliensis. To note, these OTUs were not associated with disease by Nishiyama et al. (2020).

### Example 2

### Bacteriophage Exposure Drives Phase Variation

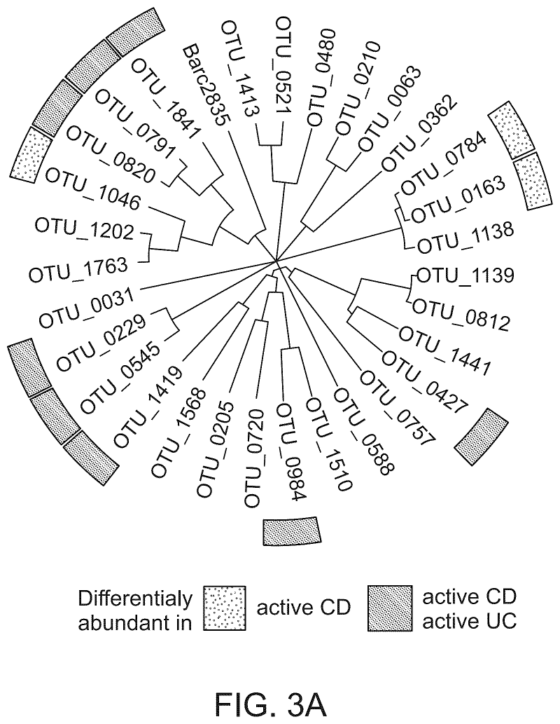

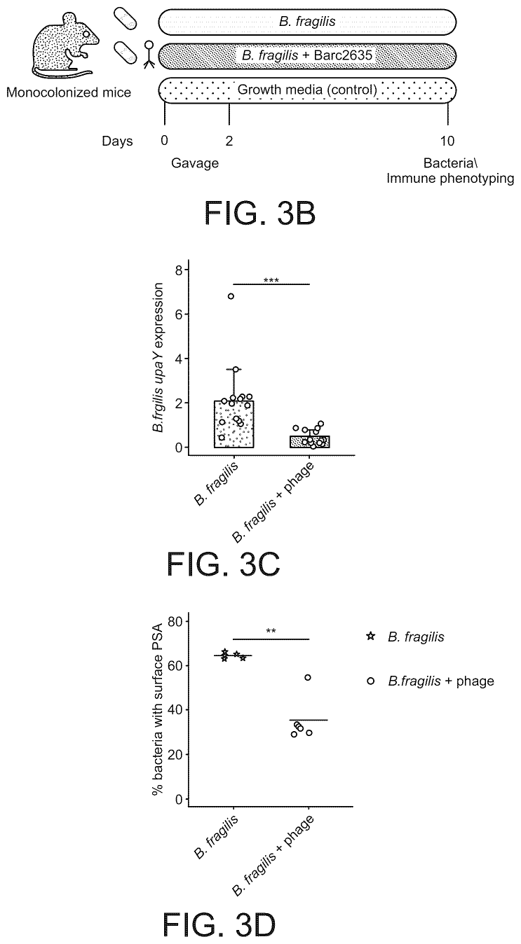

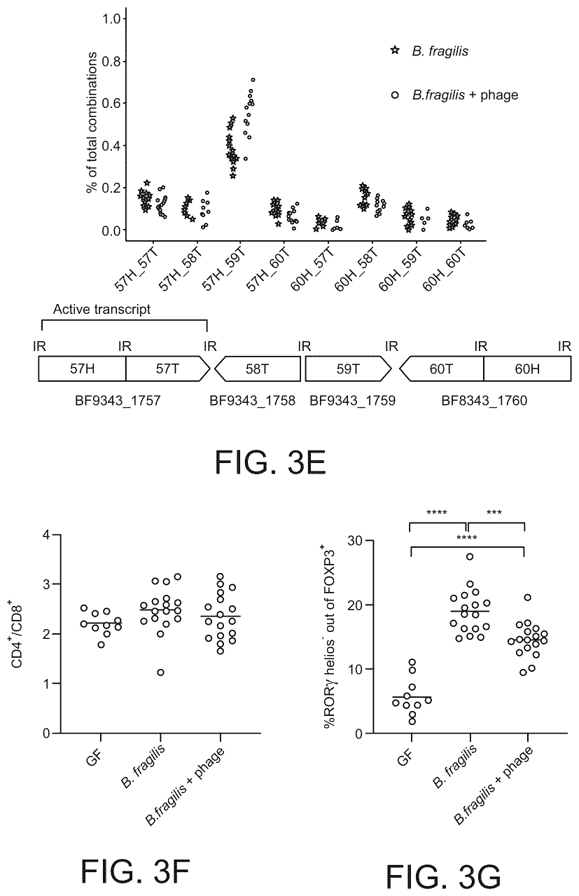

Since a higher abundance of bacteriophages were correlated with the ‘OFF’ orientation of the PSA promoter, the inventors sought to study whether encounter with bacteriophage can trigger genomic phase variation in B. fragilis. Bacteriophage Barc2635 was isolated from the sewage in Barcelona, sequenced (GenBank accession: MN078104), and characterized by Electron microscopy for its morphology (FIG. 2A), and by a plaque assay (FIG. 2B). Barc2635 is a double-stranded DNA lytic bacteriophage of 45990 bp with a GC content of 38.9%, containing 67 putative CDS belonging to the siphoviridae family and the Caudovirales order (FIG. 2C). Phage binding to cells (adsorption) rate was maximized at 20 minutes, where 67% of the phages were absorbed (FIG. 2D). One-step growth curve was carried out to determine the latent period and burst size (FIG. 2E). The phage had a burst size of about 1000 phage/cell and latency period of 35 min. Interestingly, increased abundance of bacteriophages from the Caudovirales order were found to be correlated with IBD patients (UC and CD) [Norman J M, et al. Cell. 2015; 160 (3): 447. doi: 10.1016/J.CELL.2015.01.002]. The inventors analyzed the sequence similarities of Barc2635, with the B. fragilis associated bacteriophages identified in the IBDMDB by [Nishiyama H, et al. Microorganisms. 2020; 8 (11): 1-15] and found that Barc2635 is most similar to a cluster of bacteriophages associated with an active disease including viral OTUs 0791 and 0820 (FIG. 3A). Additional bacteriophages found to be associated with bacterial population presenting the ‘ON’ orientation of PSA include viral OTU 0031, predicted to infect: M. 0101 Bacteroides uniformis and M. 2069 Bacteroides fragilis; and viral OTU 0005, predicted to infect: M. 0397 Bacteroides vulgatus, M. 0529 Bacteroides xylanisolvens, M. 1270 Bacteroides xylanisolvens, M. 3045 Bacteroides caccae. Additional bacteriophages found to be associated with the ‘OFF’ orientation include: viral OTU 0576, predicted to infect: M. 0722 Bacteroides uniformis, M. 3045 Bacteroides caccae; viral OTU 0584, predicted to infect: M. 1006 Bacteroides cellulosilyticus, M. 2826 Odoribacter splanchnicus, M. 3027 Bacteroides massiliensis, M. 3045 Bacteroides caccae, M. 3115 Bacteroides ovatus; viral OTU 1146, predicted to infect: M. 0223 Bacteroides uniformis, M. 0352 Bacteroides thetaiotaomicron, M. 0397 Bacteroides vulgatus, M. 3027 Bacteroides massiliensis; viral OTU 1125, predicted to infect: M. 0619 Bacteroides vulgatus, M. 0703 Bacteroides stercoris; and viral OTU 0802, predicted to infect: M. 0655 Bacteroides uniformis, M. 3027 Bacteroides massiliensis.

To test whether bacteriophages can induce phase variations in a controlled experiment, GF mice were monocolonized with B. fragilis NCTC 9343 in the presence or absence of bacteriophage Barc2635 and analyzed genomic-wide DNA inversion state (FIG. 3B). In the presence of Barc2635, B. fragilis exhibits continuous genomic phase variations in outersurface components such as polysaccharide utilization loci (PULs), ABC transporters and polysaccharides, including PSA. In response to the bacteriophage infection, the PSA promoter of B. fragilis phase-varied to its ‘OFF’ state, starting at day 7, with a gradual decrease in the percent of bacteria with the ‘ON’ orientation until day 42, the end of the experiment, (FIG. 4A) and, in parallel, an increase in the percent of ‘ON’ orientation of the polysaccharide F (PSF) promoter (FIG. 4B). To note, throughout this time course of 42 days Barc2635 and B. fragilis co-existed in gnotobiotic mice, with mild fluctuations of their abundances (FIG. 4C). Ten days after initial colonization, the PSA promoter activity was monitored by quantifying (qPCR) the mRNA of the first gene of the PSA biosynthesis locus, UpaY [Nishiyama H, et al. Cell. 2015; 160 (3): 447]. The mRNA of UpaY is expressed when the PSA promoter is ‘ON’, functioning as an anti-terminator of PSA biosynthesis. FIG. 3C shows a significant reduction in UpaY mRNA expression level of B. fragilis isolated from mice in the presence of bacteriophage Barc2635 in comparison to mice without the bacteriophage. To directly measure the levels of the outersurface PSA polysaccharide within the B. fragilis bacterial population, specific antibodies to PSA were used and monitored by flow cytometry. The percentage of bacterial cells expressing PSA from mice with bacteriophage Barc2635 was found to be significantly lower in comparison to bacterial cells from mice without the phage (FIG. 3D), in agreement with the UpaY mRNA expression levels. In both mice groups (i.e. B. fragilis alone vs. B. fragilis with Barc2635), the CFU levels of B. fragilis were comparable, with a 100.5 difference (FIG. 4D), and no phages were detected in the B. fragilis monocolonized group (i.e. without bacteriophage inoculation) (FIG. 4E).

In a previous study, the inventors demonstrated that phase variation in genes encoding specificity proteins for a Type I restriction-modification system (R-M), in B. fragilis, results in altered transcription of some capsular polysaccharides, including PSA, with potential altered immunomodulatory functionality of these bacteria [12]. Since the Type I R-M system are known to provide protection against bacteriophages [Noyer-Weidner M, et al. EXS. 1993; 64:39-108; Bickle T A, et al. Microbiol Rev. 1993;57 (2): 434-450; Murray N E, et al. Mol Microbiol. 1993;9 (1): 133-143; Sistla S, Rao DN. Crit Rev Biochem Mol Biol. 2004; 39 (1): 1-19], the inventors used long-reads sequencing to assess phase-variable changes in the genomic Type I R-M specificity proteins combinations upon exposure to Barc2635. The results demonstrate different patterns of specificity gene ratios in B. fragilis isolated from mice inoculated with the phage compared to those not exposed to phage (FIG. 3E).

### Example 3

### Phage Drives Reduction in T_{reg} Cells Population

Some bacterial polysaccharides were shown to be targets for bacteriophages [PMID: 32601452], but the interactions between polysaccharide-specific bacteriophages, the state of the polysaccharides and the immune system remain unclear. PSA of B. fragilis was shown to induce regulatory T cells (Tregs, CD4+Foxp3+Rorg+) in colonic lamina propria of mice [Round J L, Mazmanian S K. Proc Natl Acad Sci USA. 2010; 107 (27): 12204-12209; Erturk-Hasdemir D, et al. Proc Natl Acad Sci USA. 2019. 116 (52): 26157-26166; Johnson J L, et al. Glycobiology. 2018. 28 (1): 50-823 58; Telesford K M, et al. Gut Microbes. 2015. 6 (4): 234-242. To monitor the effect of Barc2635 during B. fragilis colonization on the CD4+Foxp3+Rorg+Tregs population, the inventors extracted cells from mice colonic lamina propria and immunophenotyped them by flow cytometry. It was found that in the presence of Barc2635 the CD4+Foxp3+Rorg+Tregs population is decreased in coherence with the decrease in the UpaY mRNA, and at the same timepoint in which the B. fragilis population displayed less PSA on its outersurface (FIG. 3F, FIG. 3G). These data link phage to alterations in bacterial functionality with concomitant effects on host physiology.

### Example 4

To further assess the effect of bacteriophages on phase variation in disease state, several phages infecting B. fragilis NCTC 9394 were isolated from feces of IBD patients (before receiving anti-TNF treatment) and their capability to alter the orientation of the PSA promoter of B. fragilis was tested in gnotobiotic mice monocolonized with B. fragilis and each of the isolated phages. More specifically, germ-free mice were gavaged with B. fragilis NCTC 9394 and after 2 days (TO) feces were collected, and the mice were further gavaged separately with each of phages isolated from IBD patients as described in the experimental procedure. The PSA orientation was tested by qPCR in feces collected at T0 and T10 (FIG. 6). As shown in FIG. 6, the inventors identified a phage, isolated from the feces of patient HR 71 V1 (plaque 1 HR 71 V1) which was shown to significantly reduce the percentage of PSA in the ‘ON’ orientation.

### Example 5

### Engineered Bacteriophages as a Tool for Modulating the Phenotype of Bacterial Populations

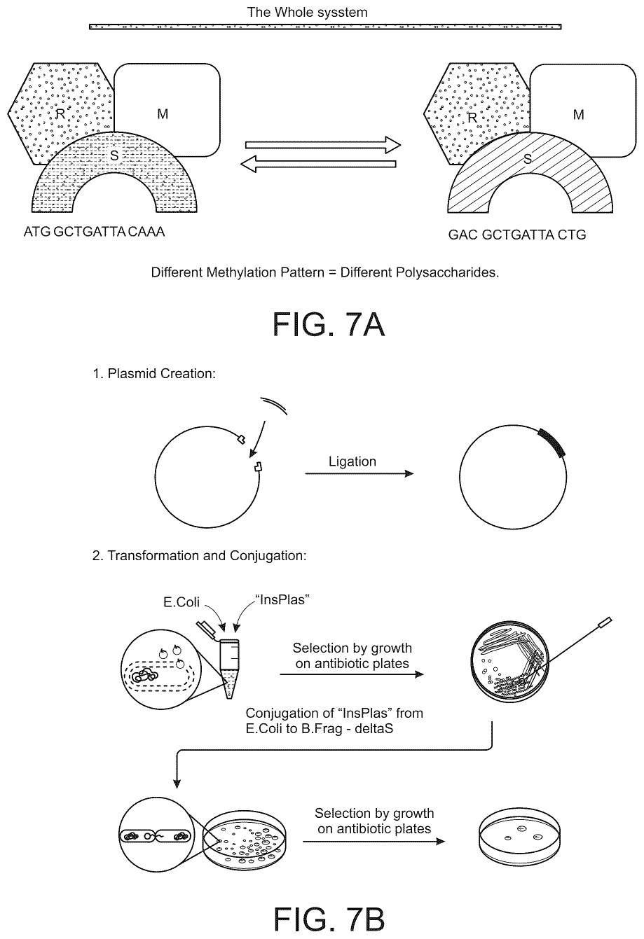

Bacteria have an arsenal of defense mechanisms against phage infection. These mechanisms are comprised of systems that are ubiquitous in bacteria and provide defense against phages and other invasive foreign DNA. The inventors are using one of these defense mechanisms, a type I restriction modification (Type1RM) system, to control bacterial phenotypes. The Type1RM is a multi-subunit enzyme. It consists of (i) an invertible specificity protein (HsdS) that recognizes foreign DNA, (ii) a Methyltransferase protein (HsdM) that methylates self-DNA to avoid self-restriction, and (iii) a restriction enzyme (HsdR) that cleaves unmethylated DNA, and as such defends the bacteria from foreign (unmethylated) DNA (FIG. 7A).

The bacteria can express only one specificity protein at a time, and by this mechanism, each bacterium has a single methylation pattern according to the specificity protein it expresses. This mechanism is controlled by DNA inversions. Each different methylation pattern creates a distinct phenotypic pattern that has been shown by the inventors to affect global transcription programs and has been related in some bacteria to their pathogenicity and ability to cause disease. This mechanism could prove as an important step while moving forward from the current state of broad-spectrum antibiotic treatments towards a more personalized functional medicine which can control bacterial phenotypes and turn pathogens to non-harmful residential bacteria. In the case of Bacteroides fragilis for example, the Type1RM system can control global transcription program and amongst them, the expression of outersurface polysaccharides [8].

The Type1RM system is unique, since it has two functions: methylation and restriction. The inventors can change the way bacteria act in a community without changing the bacterial composition itself by utilizing the Type1RM system and diverting it to express a specific methylation pattern. The inventors use bacteriophages to modulate the phenotypic landscape of the bacteria in a community. Bacteriophages are a great selection tool because they kill bacteria that cannot recognize it. The inventors are engineering bacteriophages to carry a new DNA insert that contains one of the desired specificity sequences of the Type1RM system. These bacteriophages infect and kill the bacteria which does not express this specificity protein, however bacteria which express this specificity protein will recognize the specificity sequence within the bacteriophage and cleave the bacteriophage DNA by their Type1RM restriction enzymes. In this way, the specificity sequence that will be inserted to the bacteriophage will be the only specificity sequence viable in the bacterial community, thereby enriching the bacteria which express the specificity protein and by that diverting the bacterial population to express a specific methylation pattern and specific phenotypes. This may drive a phenotypic switch, for example in the surface polysaccharides.

More specifically, the inventors are utilizing the following steps depicted in FIG. 7B. Plasmid creation: the plasmid, PFD340, and the insert are digested using the same restriction sites, and enzyme (BAMH1-HF, KpN1-Hf). Then ligated using T4 ligase enzyme. All the steps are validated using PCR.

Building plasmid that contains the insert of interest (and an antibiotic resistance gene for selection) is inserted into bacteria that successfully transformed the plasmid using relevant antibiotics: The recombinant plasmid is then transformed to E.Coli s17 lambda-pir, and grown bacteria are isolated for colony scanning using PCR. Positive colonies are then grown with a mutant of B. fragilis that does not contain the genes encoding the specific protein (i.e. the HsdS genes), which means it cannot identify foreign DNA with the type 1 R-M system and restrict it, this will allow for the uptake of the insert in a conjugation manner, and a successful infection with the bacteriophage for the next step.

B. fragilis is confirmed to contain the insert of interest and is infected with a phage specific for the B. fragilis. Based on the enormous amount of bacteria in the tube, and even larger amount of phages, it is statistically feasible that some phages took this insert and integrated it into their genome. These recombinant phages are identified by infecting multiple plates with the optimal enrichment concentration (a concentration that contains the least amount of phages detectable by PCR), and then these recombinant phages are enriched with multiple infections. The engineered bacteriophages contain a new DNA insert that upon infection activate the Type-1RM system, methylate the bacterial self protein of interest (which is encoded by the insert in the phage) and restrict the phage DNA, thus manipulating the phenotypic of the bacterial population. The first step in any genetic engineering system is generating the mutation of interest. Mutations in bacteriophage engineering occur during infection by homologous recombination between the bacteriophage DNA and a recombination template carried on a replicative plasmid inside the bacteriophage host, B. Fragilis 9343 deltaS in this case. The recombination template herein consists of two regions homologous to the phage genome regions flanking the target gene. The length of these homologous regions is 250 bp, long enough to increase the probability of homologous recombination without replicating the entire phage genome which might be lethal upon introduction to the host. A short, 108 bp, foreign DNA insert was positioned between the homologous regions that is introduced into the phage genome with minimal interruption to the compact phage genome. This insert is later further used to detect recombinant phages using designed specific primers that attach to the foreign DNA only.

Furthermore, this foreign DNA sequence contains two recognition sites for the specificity protein of the type1RM system (HsdS). The recognition sequences that the inventors chose is homologous to an invertible region found in the B. Fragilis genome and can be recognized by the specificity protein, that upon methylation alters the surface polysaccharides expressed on the bacterium and upregulates the expression of polysaccharide B (PSB) as was shown in a previous work [8]. The HsdS gene encodes a DNA-binding protein containing two target recognition domains (TRDs), each recognizes one of the two half-sites of the bipartite target, these two TRDs are separated by a conserved nucleotide sequence. The foreign DNA insert herein was designed to align with these criteria of the specificity protein, the result is a recognition site of the following sequence: GACn5CTG. This sequence is found twice in the engineered insert of foreign DNA, and two repeats were chosen in order to increase the probability of recognition by the host while keeping the sequence short enough not to disrupt compact phage genome.

The final alteration in the phage genome is an addition of 608 bp, 500 of which are homologous to the phage genome, and another 108 bp of the foreign DNA insert.

### Engineered Sequence for a Bacteriophage Comprising the Recognition Sequence for Bacteria Expressing the 57H/59T Orientation:

ccgcgcctagcacctgccgaggccgccaaGGTACCcgccataccaccggaacaccttagataaccaagcgtcaccgaacactgac
atccgttcaattacacccgtctttcgggcaaagttaacacaaaatgccgctacaaacgaaacaagtagcacgcaagaaagaaaataattata
aatctccataattcaaatattagtttgtacaaaagtatgaataaaaagcaattttgcaaagatattgattattaaaacgttagtctcgaact
ctccgaaggtggataaGACATGTTGATCTTTCGTGTTACGGGATCAATGTCAACACGTCGACCAGTT
TTAAAATCGATAAAGCCTTTTTCAACTTGTGGCGCTTGAAATTGAATGATCCTTCTGT
Caaacgaaatgtttataaataagaaaaggagtctaaaagactcctaacctacatgctttctgtataaaacgaacgcttccacaatctccggc
ataactttaagcgatccaacgataagccgccttttaatcatagacgaaataatgataatcttgtcttcttcacacegaatatatcccatctt
atcagcccacgttataaaatcaaacatttcatgttcaaacaacaaagagcctaaaggaaggtatattGGATCCagcatgcgcaacgcctgcg
agc, as denoted by SEQ ID NO: 9.
The 108 bp of the foreign DNA insert, comprises the nucleic acid sequence as denoted by SEQ
ID NO: 10:
GACATGTTGATCTTTCGTGTTACGGGATCAATGTCAACACGTCGACCAGTTTTAAAA
TCGATAAAGCCTTTTTCAACTTGTGGCGCTTGAAATTGAATGATCCTTCTGTC.

### Engineered Sequence for a Bacteriophage Comprising the Recognition Sequence for Bacteria Expressing the 57H/58T Orientation:

ccgcgcctagcacctgccgaggccgccaaGGTACCcgccataccaccggaacaccttagataaccaagcgtcaccgaacactgac
atccgttcaattacacccgtctttcgggcaaagttaacacaaaatgccgctacaaacgaaacaagtagcacgcaagaaagaaaataattata
aatctccataattcaaatattagtttgtacaaaagtatgaataaaaagcaattttgcaaagatattgattattaaaacgttagtctcgaact
ctccgaaggtggataaGACATGTTCTGTTTCGTGTTACGGGATCAATGTCAACACGTCGACCAGTTT
TAAAATCGATAAAGCCTTTTTCAACTTGTGGCGCTTGAAATTGAATCAGCTTCTGTCa
aacgaaatgtttataaataagaaaaggagtctaaaagactcctaacctacatgctttctgtataaaacgaacgcttccacaatctccggcat
aactttaagcgatccaacgataagccgccttttaatcatagacgaaataatgataatcttgtcttcttcacaccgaatatatcccatcttat
cagcccacgttataaaatcaaacatttcatgttcaaacaacaaagagcctaaaggaaggtatattGGATCCagcatgcgcaacgcctgcgag
c, as denoted by SEQ ID NO: 11, the 108 bp of the foreign DNA insert, comprises the nucleic
acid sequence as denoted by SEQ ID NO: 12:
GACATGTTCTGTTTCGTGTTACGGGATCAATGTCAACACGTCGACCAGTTTTAAAAT
CGATAAAGCCTTTTTCAACTTGTGGCGCTTGAAATTGAATCAGCTTCTGTC.

### Example 6

### Bacteriophages as a Therapeutic Tool for Colorectal Cancer (CRC)

Regulatory cells infiltration into tumor tissues is associated with a poor prognosis. There is growing evidence that reduction of Tregs enhances anti-tumor immune responses. Tumor-infiltrating Tregs are associated with an adverse outcome from most cancer types including colorectal cancer (CRC). CRC-tumors with increased numbers of T_{reg} cells have been associated with promoting tumor development, immunotherapy failure, and a poorer prognosis. Targeting tumor-associated T_{reg} cell may be an effective addition to current immunotherapy approaches. To test whether induced reduction of colonic Tregs by bacteriophages can promote increased antitumor response, CRC is induced in germ-free mice colonized with B. fragilis in comparison to B. fragilis+bacteriophage. Tumor initiation and progression are monitored by tumor size, mice weight and immunophenotyping.

1.-50. (canceled)

51. A method for modulating an immune response in a subject in need thereof, the method comprising the step of administrating to said subject at least one bacteriophage that modulates the phenotype of at least one bacteria in said subject, wherein at least one of:
(i) the bacteriophage modulates phase variation of at least one locus of said at least one bacteria; and/or
(ii) said bacteriophage comprises at least one recognition sequence for at least one bacterial restriction modification system (R-M), that is specifically recognized by at least one target recognition component of said R-M system of at least one bacterium displaying a desired phenotype; and/or
(iii) the bacteriophage modulates bacterial functionality.

52. The method according to claim 51, wherein at least one of:
(I) the method further comprising administrating said at least one bacteria to said subject;
(II) wherein said bacteria reside within at least one microbiome community of said subject; and
(III) wherein said bacteria reside within the gut microbiome.

53. The method according to claim 51, wherein at least one of:
(I) said least one bacteria comprise at least one bacterium of at least one phylum selected from Bacteroidota, Verrucomicrobiota, proteobacteria, actinobacteria, firmicutes and Tenericutes;
(II) wherein said Bacteroidota bacterium is of the genus Bacteroides, Bacteroidia, Bacteroidales, Bacteroidaceae and/or Phocaeicola;
(III) wherein said Bacteroidota bacterium comprises at least one of Bacteroides fragilis and Bacteroides thetaiotaomicron, or any isolate or species thereof; and
(IV) wherein said phase variation in at least one locus comprise phase variation/s in at least one intergenic region/s and/or intragenic region/s of said locus.

54. The method according to claim 51, wherein at least one of:
(I) said at least one locus comprises at least one locus of bacterial outersurface molecules and/or of at least one target recognition component of bacterial restriction modification system (R-M), and wherein said bacterial outersurface molecules comprise at least one capsular polysaccharide (CPS), optionally, wherein said bacterial outersurface molecules comprise capsular poly saccharides (CPS), wherein said CPS comprise 3 to 10 different CPS operons, leading to the production of distinct capsule structures or polysaccharides (PS), and wherein said PS comprise polysaccharides A to H (PSA, PSB, PSC, PSD, PSE, PSH) of distinct structures;
(II) wherein said phase variations comprise at least one inversion in at least one promoter region of at least one gene residing in said PSA and/or CPS3 and/or PSF loci, thereby converting the ON/OFF orientation of said at least one promoter region/s; and
(III) wherein said phase variations comprise at least one inversion in at least one gene of at least one target recognition component of at least one bacterial restriction modification system (R-M) thereby generating a specific combination of said gene to encode one of the target recognition components of said bacteria, having a distinct recognition site.

55. The method according to claim 54, wherein said restriction modification system is a type I restriction modification (Type1 R-M) system and wherein said at least one target recognition component of said Type1 R-M system is at least one specificity protein.

56. The method according to claim 51, wherein at least one of:
(I) said phenotype is the expression of bacterial outersurface molecules, said bacterial outersurface molecules comprise at least one of PSA, PSF, PSB and/or CPS3;
(II) said at least one bacteriophage is a natural and/or an engineered bacteriophage;
(III) said at least one bacteriophage is a bacteriophage belonging to the siphoviridae family of the Caudovirales order, and wherein said at least one bacteriophage comprises at least one of: a bacteriophage as denoted by any one of: OTU 0791, OTU 0820, OTU 1202, OTU 0165, OTU 1130, OTU 1490 of the IBDMDB database, and a bacteriophage comprising the nucleic acid sequence as denoted by SEQ ID NO: 28, designated herein as Barc2635; and
(IV) said bacteriophage is an engineered bacteriophage comprising at least one exogeneous recognition sequence of at least one bacterial restriction modification system (R-M), wherein said at least one recognition sequence is specifically recognized by at least one component of said restriction modification system of at least one bacterium displaying a desired phenotype, said recognition sequence comprises a 5′ recognition site and a 3′ recognition site separated by 3 to 10 unspecified intervening nucleotides.

57. The method according to claim 51, wherein at least one of:
(I) said modulating the immune response of a subject comprises modulating the levels of at least one of T regulatory cells and/or at least one cytokine in said subject;
(II) wherein said subject is suffering of at least one immune-related disorder; optionally, wherein said immune-related disorder is at least one of: a proliferative disorder, an inflammatory disorder, an autoimmune disorder, an immune-deficiency condition and/or an infectious disease.

58. The method according to claim 51, wherein:
(i) said subject is suffering of at least one proliferative disorder;
(ii) said modulation of the immune response of the subject comprises reduction in T regulatory cells (Tregs);
(iii) said bacteria is the gut bacteria B. fragilis; and
(iv) said desired phenotype of said bacteria comprises reduced expression of PSA and/or increased expression of PSF; optionally, wherein said bacteriophage is at least one of:
bacteriophage as denoted by any one of: OTU 0791, OTU 0820, OTU 1202, of the IBDMDB database, and bacteriophage comprising the nucleic acid sequence as denoted by SEQ ID NO: 28, designated herein as Barc2635.

59. The method according to claim 58, wherein said bacteriophage is an engineered bacteriophage comprising at least one exogeneous recognition sequence of type 1 R-M system of B. fragilis specifically recognized by a specific S protein encoded by the HsdS genes of said B. fragilis, said recognition sequence comprising: a 5′ recognition site sequence of: GAC; and a 3′ recognition site selected from: GRTY, CTG, TCC and TGC; and/or the reverse complement thereof, wherein said 5′ recognition site and a 3′ recognition site are separated by 3 to 10 intervening N nucleotides, wherein A is adenine, G is guanin, C is cytosine, T is thymine, R is adenine (A) or guanin (G), Y is cytosine (C) or thymine (T), and N is any nucleic acid residue; optionally, wherein said bacteriophage comprises at least one repeat of a recognition sequence comprising:
(a) the sequence comprising GACNNNNNGATC, and/or the reverse complement thereof comprising the sequence GATCNNNNNGTC; and/or
(b) the sequence comprising GACNNNNNCTG, and/or the reverse complement thereof comprising the sequence CAGNNNNNGTC;
wherein A is adenine, G is guanin, C is cytosine, T is thymine, R is A or G, Y is C or T, and Nis any nucleic acid residue.

60. The method according to claim 59, wherein at least one of:
(I) said engineered bacteriophage comprises an exogeneous nucleic acid sequence as denoted by SEQ ID NO: 10, or SEQ ID NO: 12, comprising at least one repeat of said at least one recognition sequence; and
(II) wherein said subject is suffering of a colorectal cancer (CRC).

61. The method according to claim 51, for treating, preventing, inhibiting, reducing, eliminating, protecting or delaying the onset of at least one immune-related disorder in a subject, the method comprising the steps of administering to said subject a therapeutically effective amount of at least one bacteriophage or any vehicle, matrix, nano- or micro-particle thereof, and/or any composition comprising the same, wherein said bacteriophage modulates the phenotype of at least one bacteria in said subject, and wherein at least one of:
(i) said bacteriophage modulates phase variation of at least one locus of said at least one bacteria; and/or
(ii) said bacteriophage comprises at least one recognition sequence for at least one bacterial restriction modification system (R-M), that is specifically recognized by at least one target recognition component of said R-M system of at least one bacterium displaying a desired phenotype; and/or
(iii) the bacteriophage modulates bacterial functionality.

62. The method according to claim 61, wherein at least one of:
(I) the method further comprising a step of administering said at least one bacteria to said subject;
(II) wherein said immune-related disorder comprises a proliferative disorder, an inflammatory disorder, an autoimmune disorder, an immune-deficiency condition and/or an infectious disease;
(III) wherein said treatment comprises modulating the immune response of the subject by the method comprising:
(i) the bacteriophage modulates phase variation of at least one locus of said at least one bacteria; and/or
(ii) said bacteriophage comprises at least one recognition sequence for at least one bacterial restriction modification system (R-M), that is specifically recognized by at least one target recognition component of said R-M system of at least one bacterium displaying a desired phenotype; and/or
(iii) the bacteriophage modulates bacterial functionality.

63. The method according to claim 61, wherein said subject is suffering of at least one proliferative disorder, and wherein the administration of said at least one bacteriophage results in the reduction in Tregs in the subject and/or in reduced expression of PSA and/or increased expression of PSF by the gut bacteria B. fragilis in said subject; optionally, wherein said bacteriophage is at least one of: bacteriophage as denoted by any one of: OTU 0791, OTU 0820, OTU 1202, of the IBDMDB database, and a bacteriophage comprising the nucleic acid sequence as denoted by SEQ ID NO: 28, also designated herein as Barc2635.

64. The method according to claim 63, wherein said bacteriophage is an engineered bacteriophage comprising at least one exogeneous recognition sequence of type 1 R-M system of B. fragilis specifically recognized by a specific S protein encoded by the HsdS genes of said B. fragilis, said recognition sequence comprising: a 5′ recognition site sequence of: GAC; and a 3′ recognition site selected from: GRTY, CTG, TCC and TGC; and/or the reverse complement thereof, wherein said 5′ recognition site and a 3′ recognition site are separated by 3 to 10 intervening N nucleotides, wherein A is adenine, G is guanin, C is cytosine, T is thymine, R is adenine (A) or guanin (G), Y is cytosine (C) or thymine (T), and N is any nucleic acid residue; optionally, at least one of:
(I) wherein said bacteriophage comprises at least one repeat of a recognition sequence comprising:
(a) the sequence comprising GACNNNNNGATC, and/or the reverse complement thereof comprising the sequence GATCNNNNNGTC; and/or
(b) the sequence comprising GACNNNNNCTG, and/or the reverse complement thereof comprising the sequence CAGNNNNNGTC;
wherein A is adenine, G is guanin, C is cytosine, T is thymine, R is A or G, Y is C or T, and Nis any nucleic acid residue; and
(II) wherein said engineered bacteriophage comprises an exogeneous nucleic acid sequence as denoted by SEQ ID NO: 10, or SEQ ID NO: 12, comprising at least one repeat of said at least one recognition sequence.

65. The method according to claim 63, wherein said subject is suffering of a colorectal cancer (CRC).

66. An engineered bacteriophage comprising at least one exogeneous recognition sequence of at least one bacterial restriction modification system (R-M), or a composition at least one of said engineered bacteriophage or any cocktail or mixture of said bacteriophages or any vehicle, matrix, nano- or micro-particle thereof; wherein said at least one recognition sequence is specifically recognized by at least one component of said restriction modification system of at least one bacterium displaying a desired phenotype, said recognition sequence comprises a 5′ recognition site and a 3′ recognition site separated by 3 to 10 unspecified intervening nucleotides, and wherein said composition further comprises at least one of pharmaceutically acceptable carrier/s, diluent/s, excipient/s and additive/s.

67. The engineered bacteriophage according to claim 66, wherein at least one of:
(I) said bacterial restriction modification system is the Type1 R-M system, and wherein said 5′ and 3′ recognition sites are recognized by the N- and C-terminal target recognition domains (TRD) of a specificity protein (S-protein) of said bacteria;
(II) wherein said bacteriophage infects at least one bacterium of the Bacteroidota phylum; optionally, at least one of:
(i) wherein said Bacteroidota bacterium is of the genus Bacteroides; and
(ii) wherein said Bacteroides bacterium is of the specie Bacteroides fragilis; and
(III) wherein said desired phenotype comprises reduced expression of PSA and/or increased expression of PSF, by said Bacteroides fragilis.

68. The engineered bacteriophage according to claim 66, wherein said bacteriophage is an engineered bacteriophage comprising at least one exogeneous recognition sequence of type 1 R-M system of B. fragilis specifically recognized by a specific S protein encoded by the HsdS genes of said B. fragilis, said recognition sequence comprising: a 5′ recognition site sequence of: GAC; and a 3′ recognition site selected from: GRTY, CTG, TCC and TGC; and/or the reverse complement thereof, wherein said 5′ recognition site and a 3′ recognition site are separated by 3 to 10 intervening N nucleotides, wherein A is adenine, G is guanin, C is cytosine, T is thymine, R is adenine (A) or guanin (G), Y is cytosine (C) or thymine (T), and N is any nucleic acid residue; optionally, at least one of:
(I) wherein said bacteriophage comprises at least one repeat of a recognition sequence comprising:
(a) the sequence comprising GACNNNNNGATC, and/or the reverse complement thereof comprising the sequence GATCNNNNNGTC; and/or
(b) the sequence comprising GACNNNNNCTG, and/or the reverse complement thereof comprising the sequence CAGNNNNNGTC;
wherein A is adenine, G is guanin, C is cytosine, T is thymine, R is A or G, Y is C or T, and Nis any nucleic acid residue; and
(II) wherein said engineered bacteriophage comprises an exogeneous nucleic acid sequence as denoted by SEQ ID NO: 10, or SEQ ID NO: 12, comprising at least one repeat of said at least one recognition sequence.

69. A method for modulating the phenotype of a bacterial population, the method comprising the step of contacting said bacterial population with an effective amount of at least one engineered bacteriophage comprising at least one exogeneous recognition sequence of at least one bacterial restriction modification system (R-M), wherein said at least one recognition sequence is specifically recognized by at least one component of said restriction modification system of at least one bacterium displaying a desired phenotype, thereby enriching bacteria displaying said desired phenotype in the bacterial population, wherein said recognition sequence comprises a 5′ recognition site and a 3′ recognition site separated by 3 to 10 unspecified intervening nucleotides.

70. The method according to claim 69, wherein at least one of:
(I) wherein at least one of:
(i) the bacteriophage modulates phase variation of at least one locus of said at least one bacteria; and/or
(ii) said bacteriophage comprises at least one recognition sequence for at least one bacterial restriction modification system (R-M), that is specifically recognized by at least one target recognition component of said R-M system of at least one bacterium displaying a desired phenotype; and/or
(iii) the bacteriophage modulates bacterial functionality;
(II) wherein said engineered bacteriophage comprising at least one exogeneous recognition sequence of at least one bacterial restriction modification system (R-M), wherein said at least one recognition sequence is specifically recognized by at least one component of said restriction modification system of at least one bacterium displaying a desired phenotype, said recognition sequence comprises a 5′ recognition site and a 3′ recognition site separated by 3 to 10 unspecified intervening nucleotides; and
(III) wherein said bacterial population is in a subject suffering from at least one immune-related disorder, optionally, said immune-related disorder is at least one of a proliferative disorder, an inflammatory disorder, an autoimmune disorder, an immune-deficiency condition and/or an infectious disease.
